(19) United States
(12) Patent Application Publication
(10) Pub. No.: US 2025/0186408 A1
(43) Pub. Date: Jun. 12, 2025

(54) TREATMENT OF PHARMACORESISTANT EPILEPSY

(71) Applicant: Katholieke Universiteit Leuven, Leuven (BE)

(72) Inventors: Peter DE WITTE, Kessel-Lo (BE); Wim Michel DE BORGGRAVE, Leuven (BE); Annelii NY, Mol (BE); Daniëlle COPMANS, Holsbeek (BE); Michèle PARTOENS, Diepenbeek (BE); Gert STEURS, Heverlee (BE); Henri Edmond Yvonne Wanda VERSCHUEREN, Oud-Heverlee (BE)

(21) Appl. No.: 18/558,423

(22) PCT Filed: May 3, 2022
(86) PCT No.: PCT/EP2022/061860

§ 371 Date: Nov. 1, 2023

(30) Foreign Application Priority Data
May 3, 2021 (EP) 21171769.9

(51) Int. Cl. A61K 31/437; A61K 31/085; A61P 25/08; C07C 43/23; C07D 471/04

(52) U.S. Cl. CPC A61K 31/437; A61K 31/085; A61P 25/08; C07C 43/23; C07D 471/04

(57) ABSTRACT

Methods for treating epilepsy, particularly a pharmacoresistant epilepsy, include administering to a subject in need thereof a compound of a class including propynones, propynals, propynols, propynes, propenones, amides, quinolinones, naphthyridinones, thiopyranoxides, pyrazolopyridines, and indazoles. The compounds have a general formula R¹—X—C≡C—R², where is selected from C═O, CH—OH, CH₂ and C═N—, with the proviso that if X is C═N—, the nitrogen is bound to a nitrogen atom of an R¹ substituent, where R¹ is hydrogen, alkyl, aromatic, or heteroaromatic, and where R² is phenyl, substituted phenyl, alkyl, or cycloalkyl.

### CROSS-REFERENCES TO RELATED APPLICATIONS

This application is a national-stage application under 35 U.S.C. § 371 of International Application No. PCT/EP2022/061860, filed May 3, 2022, which International Application claims benefit of priority to European Patent Application No. 21171769.9, filed May 3, 2021.

### TECHNICAL FIELD

The present disclosure relates to the treatment of pharmacoresistant epilepsy with propynones, propynals, propynols, propynes, propenones, amides, quinolinones, naphthyridinones, thiopyranoxides, pyrazolopyridines, and indazoles.

### BACKGROUND

More than 70 million people worldwide are affected by epilepsy, a common severe neurological disease that is characterized by an enduring predisposition of the brain to generate epileptic seizures and the associated neurobiological, cognitive, psychological, and social consequences (1-4). The first-line treatment consists of pharmacotherapy with antiseizure drugs (ASDs) to control the seizures in terms of incidence and severity (4, 5). Unfortunately, despite the availability of more than 25 approved ASDs, approximately 30% of epilepsy patients fail to achieve seizure freedom due to pharmacoresistance, also known as drug-resistance (4, 6-8). Besides, first-line ASDs are associated with important adverse effects that can significantly impact daily life (5, 9). Hence, there is an unmet medical need for safer and more effective ASDs.

The search for new and improved ASDs, effective against drug-resistant seizures, has proven particularly challenging (10). An important limitation is the fact that the more clinically-relevant animal models of drug-resistant seizures, such as the rat lamotrigine-resistant amygdala kindling model, are typically chronic models, which are not suited for drug screening because they are low-throughput and labour-intensive (11). With the exception of the pharmacoresistant acute mouse 6-Hz (44 mA) psychomotor seizure model (12), either drug-sensitive acute rodent models (e.g., the maximal electroshock seizure test (MES)) or in vitro target-based assays are used for drug screening. However, they are prone for the selection of ‘me-too’ drug candidates.

The lack of rodent models that can capture pharmacoresistant epileptic seizures and are suitable for high-throughput drug screening calls for additional drug discovery strategies to identify clinically relevant hits. In this regard the larval zebrafish model has gained interest as a small vertebrate that combines the strengths of high-throughput drug screening with in vivo testing (13, 14). Moreover, the use of a lower vertebrate for screening purposes is ethically preferable to higher vertebrates (15).

Recently, several drug-resistant larval zebrafish epilepsy and seizure models have been reported (13, 16-18). Among them is the zebrafish ethyl ketopentanoate (EKP) seizure model, a novel pharmacoresistant animal model of chemically-induced seizures that was developed by the Laboratory for Molecular Biodiscovery (Prof. P. de Witte), pharmacologically characterized, and shown to have the potential to select innovative antiseizure compounds (16). EKP is a lipid-permeable inhibitor of glutamic acid decarboxylase (GAD) that converts glutamate into γ-aminobutyric acid (GABA) (16). GAD is a key enzyme in the dynamic regulation of neural network excitability (16). Importantly, the decrease of GAD activity in zebrafish is clinically relevant as lowered GAD activity is associated with several forms of epilepsy which are often treatment resistant (19-25).

In our search for novel antiseizure compounds with the potential to treat drug-resistant seizures, we selected the zebrafish ethyl ketopentanoate (EKP) seizure model for hit identification and as critical gate-keeper for further investigation in rodent models because: (1) it is validated for the presence of behavioural and non-behavioural seizure biomarkers, (2) it has been pharmacologically characterized and demonstrated to be pharmacoresistant, (3) it has a novel and clinically relevant underlying mechanism of seizure induction, and (4) it is suitable for high-throughput screening (16, 26).

Using our zebrafish-based drug discovery approach, we found by automated behavioural analysis propynones that are active against EKP-induced drug-resistant seizures. To further investigate their antiseizure activities, improve our understanding of the structural necessities, and select hits with an optimal safety-efficacy profile, a compound library of 56 structurally related small molecules was synthesized in a systematic manner. Many of these structures are novel and/or have been synthesized for the first time. Their tolerability in zebrafish larvae was assessed and besides the initial behavioural antiseizure analysis, electrophysiological antiseizure analysis was performed using non-invasive local field potential (LFP) recordings. In total, 11 structurally related, novel classes of antiseizure compounds were discovered, namely, propynones, propynals, propynols, propynes, propenones, amides, quinolinones, naphthyridinones, thiopyranoxides, pyrazolopyridines, and indazoles, including more than 30 hits. Among the novel antiseizure compounds identified were rac-3-(4-(tert-butyl)phenyl)-1-phenylprop-2-yn-1-ol (compound 3.3) and 3-((3-chlorophenyl)ethynyl)-1H-pyrazolo[3,4-b]pyridine (compound 10.1), which were well tolerated in vivo and did not demonstrate apparent off-targets after in vitro pharmacological profiling. Moreover, their potential against drug-resistant seizures was validated in the mouse 6-Hz (44 mA) seizure model and their ADME and pharmacokinetic profiles were determined. Finally, also compounds (2-aminopyridin-3-yl)(4-(3-chlorophenyl)piperazin-1-yl)methanone (compound 6.1) and 2-(4-(tert-butyl) phenyl)-1,8-naphthyridin-4(1H)-one (compound 8.1) were selected for testing and validated in the mouse 6-Hz (44 mA) seizure model.

### SUMMARY

The limited success of current antiseizure drug therapies against pharmacoresistant epilepsy calls for new drug discovery strategies to identify clinically relevant hits. The larval zebrafish model is of particular interest as it combines the strengths of high-throughput drug screening with in vivo testing. In this study, we used the larval zebrafish ethyl ketopentanoate (EKP) seizure model, a novel animal model of drug-resistant seizures with the potential to select innovative antiseizure compounds. Here, we present the discovery of several novel classes of antiseizure compounds (i.e., propynones, propynals, propynols, propynes, propenones, amides, quinolinones, naphthyridinones, thiopyranoxides, pyrazolopyridines, and indazoles), the synthesis of a compound library of 56 (mostly novel) structurally related small molecules that resulted from a systematic behavioural antiseizure analysis using automated video tracking, their electrophysiological antiseizure analysis using non-invasive local field potential recordings, and the in-depth investigation of the novel antiseizure compounds rac-3-(4-(tert-butyl)phenyl)-1-phenylprop-2-yn-1-ol (compound 3.3) and 3-((3-chlorophenyl)ethynyl)-1H-pyrazolo[3,4-b]pyridine (compound 10.1) in terms of efficacy and safety. Compounds 3.3 and 10.1 were selected from the identified hits because they were well tolerated and highly effective. They were tested in the mouse 6-Hz (44 mA) psychomotor seizure model, a gold standard that functions as gatekeeper within the Epilepsy Therapy Screening Program (ETSP), and underwent in vitro and in vivo pharmacokinetic analysis and in vitro safety profiling. The potential of compounds 3.3 and 10.1 against drug-resistant seizures, as identified in the zebrafish EKP model, was validated in the mouse 6-Hz (44 mA) seizure model as both showed dose-dependent antiseizure activity. Moreover, among 47 common off-targets tested in vitro, no apparent off-targets were observed. Besides, also compounds (2-aminopyridin-3-yl)(4-(3-chlorophenyl)piperazin-1-yl)methanone (compound 6.1) and 2-(4-(tert-butyl)phenyl)-1,8-naphthyridin-4(1H)-one (compound 8.1) were selected for testing in the mouse 6-Hz (44 mA) seizure model, based on their structural properties, and showed dose-dependent antiseizure activity as well. Taken together, these data show that the novel classes of antiseizure compounds, identified in this study, can be of interest in the search for new and improved drug therapies against pharmacoresistant epilepsy. Continued antiseizure characterization and target identification will be key to unravel their potential. Finally, this study supports the relevance of the use of the larval zebrafish EKP seizure model as a pre-rodent animal model in drug discovery.

Embodiments herein are summarised in the following statements:
1. A compound with general structure (I) for use in the treatment of epilepsy

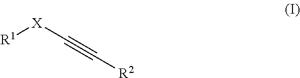

wherein X is selected from C═O, CH—OH, CH2 and C═N—, with the proviso that when X is C═N—, the nitrogen is bound to a nitrogen atom of a R¹ substituent,
wherein R¹ is selected from the group consisting of
hydrogen, methyl or a linear or branched C₂-C₄ alkyl,
an aromatic or aliphatic 5 membered ring, optionally comprising heteroatoms and/or optionally comprising further substituents,
an aromatic or aliphatic 6 membered ring, optionally comprising one or more heteroatoms and/or optionally comprising further substituents,
a double 6 membered ring, wherein one or both rings are aromatic and optionally comprising one or more hetero atoms, and/or optionally comprising further substituents, and
wherein R² is selected from the group consisting of:
phenyl, optionally further substituted with OH, OCH₃, ethyl, halomethyl, one or more halogens, or with a linear or branched C₂-C₈ alkyl, wherein the C₂-C₈ alkyl is optionally further substituted with ═O or wherein a carbon atom in the C₂-C₈ alkyl is replaced with a halogen
a linear or branched C₁-C₁₀ alkyl, a linear or branched C₁-C₈ alkyl, a linear or branched C₁-C₆ alkyl, or a C₃ to C₆ cycloalkyl, wherein in said alkyl optionally a carbon atom is replaced by a Si atom,
or a pharmaceutically acceptable salt in the form of a hydrate, solvate or complex.
2. The compound according to statement 1 for use in the treatment of epilepsy, wherein R¹ is phenyl, optionally substituted with OH, NO₂, NH₂, OCH₃, OCH₂CH₃, CH₂—NH₂, CH₂—CH₂—NH₂, or a halogen such as F or Cl.
3. The compound according to statement 1, for use in the treatment of epilepsy wherein R¹ is phenyl, substituted with a C₁-C₆, or C₁-C₄ carbon alkyl wherein carbon is optionally replaced by oxygen and/or optionally comprises one or more OH or ═O substituents.
4. The compound according to statement 1 for use in the treatment of epilepsy wherein R¹ is phenyl, substituted with aliphatic 6 membered ring, with optional 1 or 2 hetero atoms.
5. The compound according to any one of statements 1, for use in the treatment of epilepsy wherein R¹ is phenyl comprising a N atom bound to the N of the X if X is C═N—.
6. The compound according to statement 1, for use in the treatment of epilepsy wherein R¹ is an aromatic 5 membered ring, optionally comprising a sulphur heteroatom, or optionally 1 or 2 nitrogen atoms.
7. The compound according to any one of statements 1 to 6, for use in the treatment of epilepsy wherein X is selected from C═O, CH—OH and CH₂.
8. The compound according to any one of statements 1 to 6, for use in the treatment of epilepsy wherein X is selected from CH—OH.
9. The compound according statement 8, for use in the treatment of epilepsy wherein R¹ comprises a phenyl moiety and R² comprises a phenyl moiety.
10. The compound according to statement 8 or 9, for use in the treatment of epilepsy wherein R1 is phenyl and R2 is a phenyl substituted with methyl or a linear or branched C₂ to C₆ alkyl.
11. The compound according to statement 8 or 9, in the treatment of epilepsy which is rac-3-(4-(tert-butyl)phenyl)-1-phenylprop-2-yn-1-ol (3.3).
12. The compound according to any one of statements 1 to 6, for use in the treatment of epilepsy wherein X is C═N—, and the nitrogen of C═N— is bound to a N atom of R¹.
13. The compound according to statement 12, for use in the treatment of epilepsy wherein X is C═N—, and the nitrogen C═N— is bound to R1 via a N atom of a substituent on a phenyl or pyridyl moiety.
14. The compound according statement 12 or 13, for use in the treatment of epilepsy which comprises a pyrazolo [3,4-b] pyridine moiety or a indazole moiety.
15. The compound according to any one of statements 12 to 14, for use in the treatment of epilepsy which is 3-((3-chlorophenyl)ethynyl)-1H-pyrazolo[3,4-b]pyridine (10.1).
16. The compound according to any one of statements 1 to 15, for use in the treatment of epilepsy, wherein the epilepsy is a treatment resistant epilepsy.
17. A compound with general structure (I)

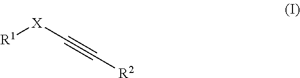

wherein X is selected from C═O, CH—OH, CH2 and C═N—, with the proviso that when X is C═N—, the nitrogen is bound to a nitrogen atom of a R¹ substituent,
wherein R¹ is selected from the group consisting of:
hydrogen, methyl or a linear or branched C₂-C₄ alkyl,
an aromatic or aliphatic 5 membered ring, optionally comprising heteroatoms and/or optionally comprising further substituents,
an aromatic or aliphatic 6 membered ring, optionally comprising one or more heteroatoms and/or optionally comprising further substituents,
a double 6 membered ring, wherein one or both rings are aromatic and optionally comprising one or more hetero atoms, and/or optionally comprising further substituents, and
wherein R² is selected from the group consisting of:
phenyl, optionally further substituted with OH, OCH₃, ethyl, halomethyl, one or more halogens, or with a linear or branched C₂-C₈ alkyl, wherein the C₂-C₈ alkyl is optionally further substituted with ═O or wherein a carbon atom in the C₂-C₈ alkyl is replaced with a halogen,
a linear or branched C₁-C₁₀ alkyl, a linear or branched C₁-C₈ alkyl, a linear or branched C₁-C₆ alkyl, or a C₃ to C₆ cycloalkyl, wherein in said alkyl optionally a carbon atom is replaced by a Si atom.
18. The compound according to statement 17, wherein R¹ is phenyl, optionally substituted with OH, NO₂, NH₂, OCH₃, OCH₂CH₃, CH₂—NH₂, CH₂—CH₂—NH₂, or a halogen such as F or Cl.
19. The compound according to statement 17, wherein R¹ is phenyl, substituted with a C₁-C₆, or C₁-C₄ carbon alkyl wherein carbon is optionally replaced by oxygen and/or optionally comprises one or more OH or ═O substituents.
20. The compound according to statement 17, wherein R¹ is phenyl, substituted with an aliphatic 6 membered ring, with optional 1 or 2 hetero atoms.
21. The compound according to statement 17, wherein R¹ is phenyl comprising a N atom bound to the N of the X if X is C═N—.
22. The compound according to statement 17, wherein R¹ is an aromatic 5 membered ring, optionally comprising a sulphur heteroatom, or optionally 1 or 2 nitrogen atoms.
23. The compound according to 17 to 23, wherein X is selected from C═O, CH—OH and CH₂.
24. The compound according to any one of statements 17 to 23, wherein X is selected from CH—OH.
25. The compound according statement 24, wherein R¹ comprises a phenyl moiety and R² comprises a phenyl moiety.
26. The compound according to statement 24 or 25, wherein R¹ is phenyl and R2 is phenyl substituted with methyl or a linear or branched C₂ to C₆ alkyl.
27. The compound according to any one of statements 24 to 26, which is rac-3-(4-(tert-butyl)phenyl)-1-phenylprop-2-yn-1-ol (3.3).
28. The compound according to any one of statements 17 to 23, wherein X is C═N—, and the nitrogen of C═N— is bound to a N atom of R¹.
29. The compound according to statement 28, wherein X is C═N—, and the nitrogen C═N— is bound to R1 via a N atom of a substituent on a phenyl or pyridyl moiety.
30. The compound according statement 28 or 29, which comprises a pyrazolo [3,4-b] pyridine moiety or a indazole moiety.
31. The compound according to any one of statements 28 to 30, which is 3-((3-chlorophenyl)ethynyl)-1H-pyrazolo[3,4-b]pyridine (10.1).

### BRIEF DESCRIPTION OF THE DRAWINGS

FIG. 1: Synthesis of ethyl ketopentanoate (EKP) via Lewis acid-catalysed allylation of ethyl glyoxylate followed by Dess-Martin oxidation. Abbreviations: DCM, dichloromethane; Dess-Martin periodinane, 3-oxo-1λ⁵-benzo[d][1,2]iodaoxole-1,1,1(3H)-triyl triacetate; EHP, ethyl hydroxypentanoate; RT, room temperature.

FIG. 2: Overview of compounds tested in FIG. 3.

FIG. 3: Behavioural antiseizure analysis of 21 compounds, including propynones, methanones, quinolin-4(1H)-ones, and 1,8-naphthyridin-4(1H)-ones, in the zebrafish EKP seizure model. Antiseizure activity of 21 compounds at their maximum tolerated concentrations after 2 h of incubation. Ethyl ketopentanoate (EKP)-induced seizure behaviour during the 30-min recording period was quantified and the data are plotted as mean actinteg per 5 min (±SD). Number of larvae per condition: 60 larvae were used for vehicle (VHC)+VHC and VHC+EKP controls and 12 for all compound+EKP conditions. Statistical analysis: one-way ANOVA with Dunnett's multiple comparison test (GraphPad Prism 8, San Diego, CA, USA). Significance levels: *p≤0.05, **p≤0.01, ***p≤0.001, ****p≤0.0001.

FIG. 4: Overview of the compound library.

FIG. 5: Behavioural antiseizure analysis of propynones, propynals, propynols, propynes, propenones, amides, quinolinones, naphthyridinones, thiopyranoxides, pyrazolopyridines, and indazoles in the zebrafish EKP seizure model. Antiseizure activity of 10 μM compound (A) and 2 μM compound (B) in the zebrafish ethyl ketopentanoate (EKP) seizure model after 2 h of incubation. EKP-induced seizure behaviour during the 30-min recording period was quantified and normalized against EKP-treated controls (vehicle (VHC)+EKP). The data are plotted as mean (±SD) percentage of EKP-induced seizure behaviour. Number of larvae per condition: (A) 384 larvae were used for VHC+VHC and VHC+EKP controls and 10 larvae for all compound+EKP conditions, except for compounds 1.1 (n=8), 1.11 (n=5), 1.13 (n=20), 1.14 (n=32), 1.16 (n=32), 1.22 (n=92), 1.24 (n=52), 6.1 (n=20), 9.1 (n=20), and 10.1 (n=20). (B) 384 larvae were used for VHC+VHC and VHC+EKP controls and 10 larvae were used for compound+EKP conditions, except for compounds 1.13 (n=20), 1.14 (n=32), 1.15 (n=32), 1.16 (n=31), 1.20 (n=20), 1.21 (n=20), 1.22 (n=94), 1.24 (n=51), 1.36 (n=8), 5.2 (n=20), 6.1 (n=20), 9.1 (n=20), and 10.1 (n=20). Statistical analysis: one-way ANOVA with Dunnett's multiple comparison test (A, B) (GraphPad Prism 9, San Diego, CA, USA). Significance levels: *p≤0.05, **p≤0.01, ***p≤0.001, ****p≤0.0001.

FIG. 6: Electrophysiological antiseizure analysis of propynones, propynals, propynols, propynes, propenones, amides, quinolinones, naphthyridinones, thiopyranoxides, pyrazolopyridines, and indazoles in the zebrafish EKP seizure model. Noninvasive local field potential recordings from the optic tectum of larvae pre-exposed to vehicle (VHC) and ethyl ketopentanoate (EKP), VHC only, and compound and EKP. Larvae were incubated with 10 μM of compound (A) or 2 μM of compound (B) for 22±1 h. The epileptiform brain activity of zebrafish larvae was recorded for a period of 10 min and quantified via power spectral density (PSD) analysis. The PSD ranging from 20-90 Hz is normalized against VHC-treated controls (VHC+VHC) and the data are plotted as mean (±SEM) PSD per larva. Number of larvae per condition: (A) 27 larvae were used for VHC+VHC controls, 23 larvae were used for VHC+EKP controls, and 6-15 larvae were used for compound+EKP conditions, (B) 50 larvae were used for VHC+VHC controls, 57 larvae were used for VHC+EKP controls, and 6-14 larvae were used for compound+EKP conditions. Statistical analysis: one-way ANOVA with Dunnett's multiple comparison test (A, B), outliers were identified via the ROUT test (Q=1%) (GraphPad Prism 9, San Diego, CA, USA). Significance levels: *p≤0.05, **p≤0.01, ***p≤0.001, ****p≤0.0001.

FIG. 7: Behavioural antiseizure analysis of compounds 3.3, 10.1, 6.1 and 8.1 in the mouse 6-Hz (44 mA) psychomotor seizure model. Drug-resistant psychomotor seizures were induced by electrical stimulation (6 Hz, 0.2 ms rectangular pulse width, 3 s duration, 44 mA) through the cornea, 30 min after i.p. injection of vehicle (VHC), positive control valproate (VPA), compound 3.3, compound 10.1, compound 6.1 or compound 8.1. Number of mice per condition: (A, B) 13 mice were used for VHC controls, 6 mice were used for VPA controls and 6-7 mice were used for the different compound 3.3 conditions, (C, D) 15 mice were used for VHC controls, 6 mice were used for VPA controls and 5-6 mice were used for the different compound 10.1 conditions. (E, F) 10 mice were used for VHC controls and 6 mice were used for the different compound 6.1 conditions. (G, H) 10 mice were used for VHC controls and 5-6 mice were used for the different compound 8.1 conditions. Mean seizure durations (±SD) are depicted. Statistical analysis: one-way ANOVA with Dunnett's multiple comparison test (GraphPad Prism 9, San Diego, CA, USA). Significance levels: *p≤0.05, **p≤0.01, ***p≤0.001, ****p≤0.0001.

FIG. 8: Pharmacokinetic analysis of compound 3.3 (A, B) and compound 10.1 (C, D) in naïve mice. Mean (±SD) plasma (A, C) and brain (B, D) concentrations are given at different time points after a single i.p. administration of 300 mg/kg test compound. Number of mice per condition: (A, C) 3-4 mice were used for plasma concentration calculations for all time points, except for 24 h (n=1). (B, D) 3-4 mice were used for brain concentration calculations for all time points, except for 24 h (n=1) and for 2 min (n=2-3).

### DETAILED DESCRIPTION

The present disclosure relates to compounds and their use in the treatment of epilepsy, in particular drug-resistant epilepsy.

A first aspect relates to compounds and their use in the treatment of epilepsy, in particular drug-resistant epilepsy with general structure (I)

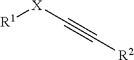

Herein X is selected from C═O, CH—OH and CH₂.

Furthermore X can be C═N—, whereby the N atom of C═N— is bound to a N atom of a R¹. When X is C═N—R¹ is has an amino phenyl, or amino pyridyl moiety, typically an 2-amino phenyl, or 2-amino pyridyl moiety. More specifically R¹ is 2-amino phenyl, or 2-amino pyridyl.

In compounds with general structure (I), R¹ is selected from the group consisting of
hydrogen, a linear or branched C₂-C₆ alkyl, a linear or branched C₂-C₄ alkyl,
linear or branched C₂-C₆ alkene, a linear or branched C₂-C₄ alkene, linear or branched C₂-C₆ alkyne, a linear or branched C₂-C₄ alkyne,
an aromatic or aliphatic 5 membered ring, optionally comprising heteroatoms and/or optionally comprising further substituents,
an aromatic or aliphatic 6 membered ring, optionally comprising one or more heteroatoms and/or optionally comprising further substituents,
a double 6 membered ring, wherein one or both rings are aromatic and optionally comprise one or more hetero atoms.

In embodiments hereof, R¹ is phenyl, optionally substituted with OH, NO2, NH₂, OCH₃, CH₂—NH₂, CH₂—CH₂—NH2, and a halogen.

In embodiments hereof, R¹ is phenyl, substituted with a C₁-C₆ carbon alkyl wherein carbon is optionally replaced by oxygen and/or optionally comprise OH or ═O substituents. Examples of such substituents are methyl and —(C═O)—O—CH₂—CH₃.

In embodiments hereof, R¹ is phenyl, substituted with an aliphatic 6 membered ring, with optional 1 or 2 hetero atoms.

In specific embodiments the phenyl has an N containing substituent bound to the nitrogen of C═N— in the general formula (I).

In embodiments hereof, R¹ is an aromatic 5 membered ring, optionally comprising a sulphur heteroatom, or optionally comprising 1 or 2 nitrogen hetero atoms. The substituents are typically at the 2, 4, or 6 position.

In specific embodiments, substituents on the R¹ phenyl results in benzocyclohexyl optionally comprising 1 or 2 oxygen heteroatoms (as indicated in compound 1.3.

In C₂-C₆ carbon alkyls optionally carbon is replaced by oxygen and/or comprise OH or ═O substituents.

Examples of R¹ are hydrogen, [1,4]dioxin-6-yl, 1H-imidazol-2-yl, 2-(dimethylamino) pyridin-3-yl), 2-(methylamino)pyridin-3-yl), 2,3-dihydrobenzo[b], 2-amino-5-methylphenyl, 2-aminophenyl, 2-aminopyridin-3-yl, 2-hydroxyphenyl, 2-methoxyphenyl, 2-morpholinopyridin-3-yl, 3-aminopyridin-2-yl, 3-fluorophenyl, 4-(ethoxycarbonyl), 4-aminophenyl, 4-methoxyphenyl, 4-methylpyridin-3-yl, 4-nitrophenyl, isoquinolin-4-yl, phenyl, pyridin-3-yl, and thiophen-3-yl.

In the compounds with general structure (1), R² is a phenyl, optionally comprising one or two hetero atoms and/or optionally further substituted with OH, OCH₃, methyl, halomethyl, one or more halogens, or with a linear or branched C₂-C₈ alkyl, optionally further substituted with ═O or wherein a carbon atom in the C₂-C₈ is replace with a halogen.

R² can be a linear or branched C₁-C₁₀ alkyl, C₁-C₈ alkyl, C₁-C₆ alkyl, C₁-C₁₀ alkene, C₁-C₈ alkene, C₁-C₆ alkene, C₁-C₁₀ alkyne, C₁-C₈ alkyne, C₁-C₆ alkyne, wherein optionally a carbon atom is replaced by a Si atom. R² can be a C₃ to C₆ cycloalkyl, such a cyclopropyl.

Examples of R² are 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

In specific embodiments the compounds and their use in the treatment of epilepsy, in particular drug-resistant epilepsy are propynones, wherein X in general structure (I) is C=0.

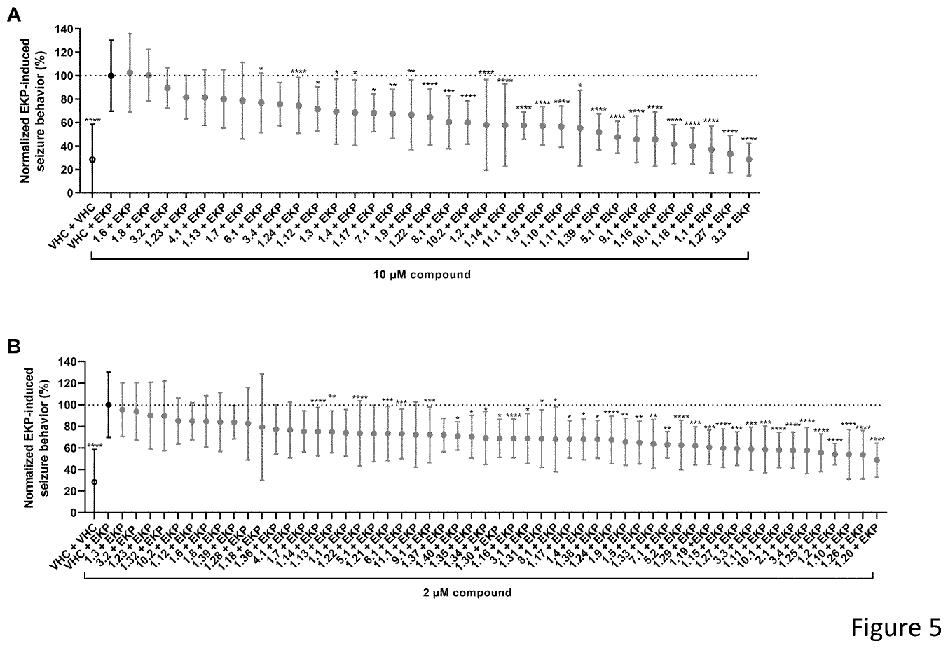

Examples hereof are compounds 1.1. to 1.41 depicted in FIG. 4.

In specific embodiments compounds for the treatment of epilepsy, in particular drug-resistant epilepsy are 1, 2, 3, 5, 6, 7, 8, 9, 10, 15, 20, 25 compounds selected from the group consisting of 1-(4-methoxyphenyl)-3-(p-tolyl)prop-2-yn-1-one (I.1), 1-(4-nitrophenyl)-3-(p-tolyl)prop-2-yn-1-one (I.2), 1-(4-aminophenyl)-3-(4-(tert-butyl)phenyl)prop-2-yn-1-one (I.4), 1-(2-aminophenyl)-3-(4-(tert-butyl)phenyl)prop-2-yn-1-one (I.5), 3-(4-(tert-butyl)phenyl)-1-(thiophen-3-yl)prop-2-yn-1-one (I.8), 1-(thiophen-3-yl)non-2-yn-1-one (I.9), 3-cyclopropyl-1-(thiophen-3-yl)prop-2-yn-1-one (I.10), 3-(4-(tert-butyl)phenyl)-1-(4-methylpyridin-3-yl)prop-2-yn-1-one (I.11), and 3-(4-(tert-butyl)phenyl)-1-(2-(methylamino)phenyl)prop-2-yn-1-one (I.14) 1-(2-aminopyridin-3-yl)-3-phenylprop-2-yn-1-one (1.1), 1-(2-aminopyridin-3-yl)-3-(4-fluorophenyl)prop-2-yn-1-one (1.2), 1-(2-aminopyridin-3-yl)-3-(triisopropylsilyl)prop-2-yn-1-one (1.3), 1-(2-aminopyridin-3-yl)-3-cyclohexylprop-2-yn-1-one (1.4), 1-(2-aminopyridin-3-yl)oct-2-yn-1-one (1.5), 1-(2-aminopyridin-3-yl)-4-methylpent-2-yn-1-one (1.6), 1-(2-aminopyridin-3-yl)-3-(4-chlorophenyl)prop-2-yn-1-one (1.7), methyl 4-(3-(2-aminopyridin-3-yl)-3-oxoprop-1-yn-1-yl)benzoate (1.8), 1-(2-aminopyridin-3-yl)-3-(4-methoxyphenyl)prop-2-yn-1-one (1.9), 1-(2-aminopyridin-3-yl)-3-(3-chlorophenyl)prop-2-yn-1-one (1.10), 1-(2-aminopyridin-3-yl)-3-(4-(trifluoromethyl)phenyl)prop-2-yn-1-one (1.11), 1-(2-aminopyridin-3-yl)-3-(p-tolyl)prop-2-yn-1-one (1.12), 1-(2-aminopyridin-3-yl)-3-(3,4-dichlorophenyl)prop-2-yn-1-one (1.13), 1-(2-aminophenyl)-3-(4-(tert-butyl)phenyl)prop-2-yn-1-one (1.14), 3-(4-(tert-butyl)phenyl)-1-(pyridin-3-yl)prop-2-yn-1-one (1.15), 3-(4-(tert-butyl)phenyl)-1-phenylprop-2-yn-1-one (1.16), 1-(2-aminopyridin-3-yl)-3-(3-(trifluoromethyl)phenyl)prop-2-yn-1-one (1.17), 3-(4-(tert-butyl)phenyl)-1-(2-methoxyphenyl)prop-2-yn-1-one (1.18), 3-(4-(tert-butyl)phenyl)-1-(2-hydroxyphenyl)prop-2-yn-1-one (1.19), 3-(3-chlorophenyl)-1-(2-chloropyridin-3-yl)prop-2-yn-1-one (1.20), 3-(3-chlorophenyl)-1-(2-morpholinopyridin-3-yl)prop-2-yn-1-one (1.21), 1-(2-aminopyridin-3-yl)-3-(4-(tert-butyl)phenyl)prop-2-yn-1-one (1.22), 1-(2-aminopyridin-3-yl)non-2-yn-1-one (1.23), 1-(3-aminopyridin-2-yl)-3-(4-(tert-butyl)phenyl)prop-2-yn-1-one (1.24) 3-(3-chlorophenyl)-1-(pyridin-3-yl)prop-2-yn-1-one (1.25), 3-(3-chlorophenyl)-1-(1H-imidazol-2-yl)prop-2-yn-1-one (1.26), 3-(3-chlorophenyl)-1-(2-(dimethylamino)pyridin-3-yl)prop-2-yn-1-one (1.27), 1,3-diphenylprop-2-yn-1-one (1.28), 3-(4-methoxyphenyl)-1-phenylprop-2-yn-1-one (1.29), 1-phenyl-3-(p-tolyl)prop-2-yn-1-one (1.30), 3-(4-chlorophenyl)-1-phenylprop-2-yn-1-one (1.31), 3-(3-methoxyphenyl)-1-phenylprop-2-yn-1-one (1.32), 1-phenyl-3-(m-tolyl)prop-2-yn-1-one (1.33), 3-(3-chlorophenyl)-1-phenylprop-2-yn-1-one (1.34) 3-(3-fluorophenyl)-1-phenylprop-2-yn-1-one (1.35), 3-(2-methoxyphenyl)-1-phenylprop-2-yn-1-one (1.36), 1-phenyl-3-(o-tolyl)prop-2-yn-1-one (1.37), 3-(2-chlorophenyl)-1-phenylprop-2-yn-1-one (1.38), 3-(4-(tert-butyl)phenyl)-1-(3-fluorophenyl)prop-2-yn-1-one (1.39), 1-(2-aminophenyl)-3-(3-chlorophenyl)prop-2-yn-1-one (1.40), 1-(2-chlorophenyl)-3-(3-chlorophenyl)prop-2-yn-1-one (1.41).

In specific embodiments the compounds and their use in the treatment of epilepsy, in particular drug resistant epilepsy are propynals, wherein X in general structure (I) is C=0 and R¹ is hydrogen.

An example hereof is compound 2.1. as depicted in FIG. 4.

In specific embodiments the compounds and their use in the treatment of epilepsy, in particular drug-resistant epilepsy are propynols, wherein X in general structure (I) is CH—OH.

Examples hereof are compounds 3.1. to 3.4 in FIG. 4.

In specific embodiments the compounds and their use in the treatment of epilepsy, in particular drug-resistant epilepsy are propynes, wherein X is CH₂.

An example hereof is compound 4.1 as depicted in FIG. 4.

A second aspect relates to propenones for the treatment of epilepsy, in particular drug-resistant epilepsy with general structure (II)

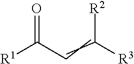

Herein R¹ and R² are as defined for formula I of the first aspect, and R³=R².

Examples, of R¹ are hydrogen, methyl, ethyl, [1,4]dioxin-6-yl, 1H-imidazol-2-yl, 2-(dimethylamino), 2-(methylamino), 2,3-dihydrobenzo[b], 2-amino-5-methylphenyl, 2-aminophenyl, 2-aminopyridin-3-yl, 2-hydroxyphenyl, 2-methoxyphenyl, 2-morpholinopyridin-3-yl, 3-aminopyridin-2-yl, 3-fluorophenyl, 4-(ethoxycarbonyl), 4-aminophenyl, 4-methoxyphenyl, 4-methylpyridin-3-yl, 4-nitrophenyl, isoquinolin-4-yl, phenyl, pyridin-3-yl, and thiophen-3-yl.

Examples of R² and R³ are independently selected from the group consisting of 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

Examples hereof are compounds 5.1 and 5.2 depicted in FIG. 4.

A third aspect relates to amides for the treatment of epilepsy, in particular drug-resistant epilepsy with general structure (III)

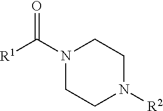

Herein R¹ and R² are as defined for formula I of the first aspect,

Examples of R¹ are hydrogen, methyl, ethyl, [1,4]dioxin-6-yl, 1H-imidazol-2-yl, 2-(dimethylamino) pyridin-3-yl), 2-(methylamino) pyridin-3-yl), 2,3-dihydrobenzo[b], 2-amino-5-methylphenyl, 2-aminophenyl, 2-aminopyridin-3-yl, 2-hydroxyphenyl, 2-methoxyphenyl, 2-morpholinopyridin-3-yl, 3-aminopyridin-2-yl, 3-fluorophenyl, 4-(ethoxycarbonyl), 4-aminophenyl, 4-methoxyphenyl, 4-methylpyridin-3-yl, 4-nitrophenyl, isoquinolin-4-yl, phenyl, pyridin-3-yl, and thiophen-3-yl.

Examples of R² are 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

An example hereof is compound 6.1 depicted in FIG. 4.

A fourth aspect relates to compounds and their use in the treatment of epilepsy, in particular drug-resistant epilepsy with general structure (IV)

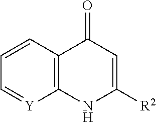

Herein Y is nitrogen or carbon,

Herein R² is as defined for formula (I) of the first aspect,

Examples of R² are 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

Examples hereof are quinoline-4-(1H)-ones such as compound 7.1 depicted in FIG. 4.

Examples hereof are 1s,8-Naphthyridin-4-(1H)-ones such as compound 8.1 depicted in FIG. 4.

A sixth aspect relates to thiopyran 1-oxides for the treatment of epilepsy, in particular drug-resistant epilepsy, with general structure (V)

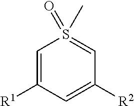

Herein R¹ and R² are as defined for formula I of the first aspect,

Examples of R¹ are hydrogen, methyl, ethyl, [1,4]dioxin-6-yl, 1H-imidazol-2-yl, 2-(dimethylamino) pyridin-3-yl), 2-(methylamino) pyridin-3-yl), 2,3-dihydrobenzo[b], 2-amino-5-methylphenyl, 2-aminophenyl, 2-aminopyridin-3-yl, 2-hydroxyphenyl, 2-methoxyphenyl, 2-morpholinopyridin-3-yl, 3-aminopyridin-2-yl, 3-fluorophenyl, 4-(ethoxycarbonyl), 4-aminophenyl, 4-methoxyphenyl, 4-methylpyridin-3-yl, 4-nitrophenyl, isoquinolin-4-yl, phenyl, pyridin-3-yl, and thiophen-3-yl.

Examples of R² are 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

An example hereof is compound 9.1. depicted in FIG. 4.

A seventh aspect relates to compounds and their use in the treatment of epilepsy, in particular drug-resistant epilepsy with general structure (VI)

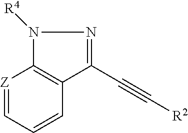

Wherein Z is carbon or nitrogen,

R⁴ are the same R¹ as defined for formula (I) of the first aspect,

Examples of R⁴ are hydrogen, methyl, ethyl, [1,4]dioxin-6-yl, 1H-imidazol-2-yl, 2-(dimethylamino) pyridin-3-yl), 2-(methylamino) pyridin-3-yl), 2,3-dihydrobenzo[b], 2-amino-5-methylphenyl, 2-aminophenyl, 2-aminopyridin-3-yl, 2-hydroxyphenyl, 2-methoxyphenyl, 2-morpholinopyridin-3-yl, 3-aminopyridin-2-yl, 3-fluorophenyl, 4-(ethoxycarbonyl), 4-aminophenyl, 4-methoxyphenyl, 4-methylpyridin-3-yl, 4-nitrophenyl, isoquinolin-4-yl, phenyl, pyridin-3-yl, thiophen-3-yl, methyl, and alkyl.

R² is as defined for formula (I) of the first aspect,

Example of R² are 2-chlorophenyl, 2-methoxyphenyl, 3-(trifluoromethyl), 3,4-dichlorophenyl, 3-chlorophenyl, 3-fluorophenyl, 3-methoxyphenyl, 4-(methoxycarbonyl), 4-(tert-butyl)phenyl, 4-(trifluoromethyl), 4-chlorophenyl, 4-fluorophenyl, 4-methoxyphenyl, cyclohexyl, cyclopropyl, isopropyl, m-tolyl, n-hexyl, n-pentyl, o-tolyl, phenyl, and p-tolyl.

Examples hereof are pyrazolo-[3,4-b] pyridines such as compounds 10.1 and 10.2 depicted in FIG. 4.

Other examples hereof are indazoles such as compound 11.1 depicted in FIG. 4.

The present disclosure further relates to methods of treatment comprising an effective amount of a compound according to any one of the above aspects to an individual suffering from epilepsy, more particular drug resistant epilepsy.

The propynones, propynals, propynols and propynes depicted in FIG. 4, all have a triple bond, which is also present in pyrazolo [3,4-b]pyridines and indazoles.

Pyrazolo [3,4-b]pyridines and indazoles are structurally related in that the oxygen, hydroxyl or hydrogen in the propynones, propynals, propynols and propynes and is replaced by a nitrogen forming a bond with a nitrogen R¹ substituents, thereby forming a five membered ring with two nitrogens.

“Seizure” refers to a brief episode of signs or symptoms due to abnormal excessive or synchronous neuronal activity in the brain. The outward effect can vary from uncontrolled jerking movement (tonic-clonic seizure) to as subtle as a momentary loss of awareness (absence seizure).

Seizure types are typically classified on observation (clinical and EEG) rather than the underlying pathophysiology or anatomy.
I Focal seizures (Older term: partial seizures)
IA Simple partial seizures—consciousness is not impaired
IA1 With motor signs
IA2 With sensory symptoms
IA3 With autonomic symptoms or signs
IA4 With psychic symptoms
IB Complex partial seizures—consciousness is impaired (Older terms: temporal lobe or psychomotor seizures)
IB1 Simple partial onset, followed by impairment of consciousness
IB2 With impairment of consciousness at onset
IC Partial seizures evolving to secondarily generalized seizures
IC1 Simple partial seizures evolving to generalized seizures
IC2 Complex partial seizures evolving to generalized seizures
IC3 Simple partial seizures evolving to complex partial seizures evolving to generalized seizures
II Generalized seizures
IIA Absence seizures (Older term: petit mal)
IIA1 Typical absence seizures
IIA2 Atypical absence seizures
IIB Myoclonic seizures
IIC Clonic seizures
IID Tonic seizures
IIE Tonic-clonic seizures (Older term: grand mal)
IIF Atonic seizures
III Unclassified epileptic seizures

A more recent classification is published in Fisher et al. (2017) Epilepsia 58(4), 522-530.

“Epilepsy” is a condition of the brain marked by a susceptibility to recurrent seizures. There are numerous causes of epilepsy including, but not limited to birth trauma, perinatal infection, anoxia, infectious diseases, ingestion of toxins, tumours of the brain, inherited disorders or degenerative disease, head injury or trauma, metabolic disorders, cerebrovascular accident and alcohol withdrawal.

A large number of subtypes of epilepsy have been characterized and categorized. The classification and categorization system, that is widely accepted in the art, is that adopted by the International League Against Epilepsy's (“ILAE”) Commission on Classification and Terminology [See e.g., Berg et al. (2010), “Revised terminology and concepts for organization of seizures,” Epilepsia, 51(4), 676-685]:
I. Electrochemical syndromes (arranged by age of onset):
   I.A. Neonatal period: Benign familial neonatal epilepsy (BFNE), Early myoclonic encephalopathy (EME); Ohtahara syndrome
   I.B. Infancy: Epilepsy of infancy with migrating focal seizures; West syndrome; Myoclonic epilepsy in infancy (MEI); Benign infantile epilepsy; Benign familial infantile epilepsy; Dravet syndrome; Myoclonic encephalopathy in non-progressive disorders
   I.C. Childhood: Febrile seizures plus (FS+) (can start in infancy); Panayiotopoulos syndrome; Epilepsy with myoclonic atonic (previously astatic) seizures; Benign epilepsy with centrotemporal spikes (BECTS); Autosomal-dominant nocturnal frontal lobe epilepsy (ADNFLE); Late onset childhood occipital epilepsy (Gastaut type); Epilepsy with myoclonic absences; Lennox-Gastaut syndrome; Epileptic encephalopathy with continuous spike-and-wave during sleep (CSWS), also known as Electrical Status Epilepticus during Slow Sleep (ESES); Landau-Kleffner syndrome (LKS); Childhood absence epilepsy (CAE)
   I.D. Adolescence-Adult: Juvenile absence epilepsy (JAE); Juvenile myoclonic epilepsy (JME); Epilepsy with generalized tonic-clonic seizures alone; Progressive myoclonus epilepsies (PME); Autosomal dominant epilepsy with auditory features (ADEAF); Other familial temporal lobe epilepsies
   I.E. Less specific age relationship: Familial focal epilepsy with variable foci (childhood to adult); Reflex epilepsies
II. Distinctive constellations
   II.A. Mesial temporal lobe epilepsy with hippocampal sclerosis (MTLE with
   II.B. Rasmussen syndrome
   II.C. Gelastic seizures with hypothalamic hamartoma
   II.D. Hemiconvulsion-hemiplegia-epilepsy
   E. Epilepsies that do not fit into any of these diagnostic categories, distinguished on the basis of presumed cause (presence or absence of a known structural or metabolic condition) or on the basis of Primary mode of seizure onset (generalized vs. focal)
III. Epilepsies attributed to and organized by structural-metabolic causes
   III.A. Malformations of cortical development (hemimegalencephaly, heterotopias, etc.)
   III.B. Neurocutaneous syndromes (tuberous sclerosis complex, Sturge-Weber, etc.)
   III.C. Tumour
   III.D. Infection
   III.E. Trauma
IV. Angioma
   IV.A. Perinatal insults
   IV.B. Stroke
   IV.C. Other causes
V. Epilepsies of unknown cause
Vi. Conditions with epileptic seizures not traditionally diagnosed as forms of epilepsy per se
   VI.A. Benign neonatal seizures (BNS)
   VI.B. Febrile seizures (FS)

A more recent classification can be found in Scheffer et al. (2017) Epilepsia. 58, 512-521.

“Drug-resistant epilepsy (DRE)” is defined by Kwan et al. (2010) Epilepsia 52(6), 1069-1077, as “failure of adequate trials of two tolerated and appropriately chosen and used antiepileptic drugs (AED schedules) (whether as monotherapies or in combination) to achieve sustained seizure freedom.”

More particularly Drug-resistant epilepsy (DRE) is an epilepsy wherein two of the below list fails to achieve sustained seizure freedom.

A non-exhaustive list of anti-epileptic compounds includes Paraldehyde; Stiripentol; Barbiturates (such as Phenobarbital, Methylphenobarbital, Barbexaclone; Benzodiazepines (such as Clobazam, Clonazepam, Clorazepate, Diazepam Midazolam and Lorazepam); Potassium bromide; Felbamate; Carboxamides (such as Carbamazepine Oxcarbazepine and Eslicarbazepine acetate); fatty-acids (such as valproic acid, sodium valproate, divalproex sodium, Vigabatrin, Progabide and Tiagabine); Topiramate; Hydantoins (such as Ethotoin, Phenytoin, Mephenytoin and Fosphenytoin); Oxazolidinediones (such as Paramethadione Trimethadione and Ethadione); Beclamide; Primidone; Pyrrolidines such as Brivaracetam Etiracetam Levetiracetam; Seletracetam; Succinimides (such as Ethosuximide, Phensuximide and Mesuximide); Sulfonamides (such as Acetazolamide, Sultiame Methazolamide and Zonisamide); Lamotrigine; Pheneturide; Phenacemide; Valpromide; Valnoctamide; Perampanel; Stiripentol; Pyridoxine.

The compound as claimed and their use refers to the chemical formula with general structure as defined, and pharmaceutically accepted derivatives thereof. These may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form of a pro-drug or pre-drug, such as an ester. As used herein and unless otherwise stated, the term “solvate” includes any combination which may be formed by a pharmaceutical composition of this disclosure with a suitable inorganic solvent (e.g. hydrates) or organic solvent, such as but not limited to alcohols, ketones, esters, and the like. Such salts, hydrates, solvates, etc. and the preparation thereof will be clear to the skilled person.

“treatment” relates to any medical benefit and in the context of epilepsy to less severe seizures shorter seizure periods or a reduced frequency of seizures.

In the present disclosure a zebrafish model is used as a model for drug-resistant epilepsy. The lipid-permeable glutamic acid decarboxylase (GAD)-inhibitor, ethyl ketopentanoate (EKP), is used that induces drug-resistant seizures in zebrafish. GAD, converting glutamate into GABA, is a key enzyme in the dynamic regulation of neural network excitability. Clinical evidence has shown that lowered GAD activity is associated with several forms of epilepsy that are often treatment resistant. This EKP-induced epilepsy zebrafish model has been validated as a model for drug-resistant epilepsy and was used to demonstrate anticonvulsant activity of various anti-epileptic drugs (AEDs).

### EXAMPLES

### Example 1

### Compound Synthesis

### Compound Library

The compound library was synthesized by the laboratory of MolDesignS (Prof. W. De Borggraeve) using a variety of synthetic strategies. Each compound was designed based on the potency of the prior candidates, which was determined via automated behavioural antiseizure analysis. All synthetic protocols are described in detail in examples 14-27.

### Ethyl Ketopentanoate

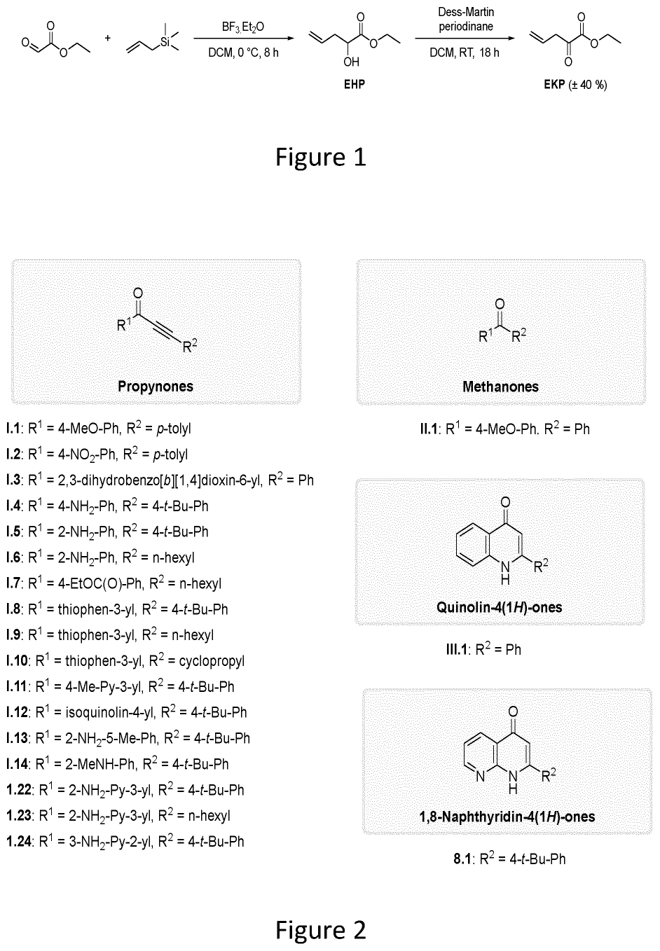

EKP was synthesized in several batches by the laboratory of MolDesignS (Prof. W. De Borggraeve), using an in-house-optimized literature procedure (16) (FIG. 1).

### Example 2

### Compound Preparation

For experiments with zebrafish larvae, dry compounds were dissolved in 100% dimethyl sulfoxide (DMSO, spectroscopy grade, Acros Organics (Geel, Belgium)) as 100-fold concentrated stocks and diluted in embryo medium to a final concentration of 1% DMSO content. Control groups were treated with 1% DMSO (VHC) in accordance with the final solvent concentration of tested compounds. For mouse experiments, a mixture of 8% solutol, 12% polyethylene glycol M.W. 200 (PEG200, >95% purity, Acros Organics (Geel, Belgium)) and 80% water was used as solvent and VHC. First, compounds were dissolved in 40% solutol/60% PEG200, after which the solution or suspension was diluted 5-fold in water. Valproate (sodium valproate, ≥98% purity) was purchased from Sigma-Aldrich (Overijse, Belgium).

### Example 3

### Experimental Animals

All animal experiments carried out were in accordance with Directive 2010/63/EU, implemented in 2020 by the Commission Implementing Decision (EU) 2020/569, and approved by the Ethics Committee of the University of Leuven (approval numbers 023/2017 and 027/2017) and by the Belgian Federal Department of Public Health, Food Safety & Environment (approval number LA1210261).

### Zebrafish

Adult zebrafish (Danio rerio) stocks of AB strain (Zebrafish International Resource Center, Oregon, WA, USA) were maintained at 28° C., on a 14/10 h light/dark cycle under standard aquaculture conditions. Fertilized eggs were collected via natural spawning and raised in embryo medium (1.5 mM HEPES, pH 7.2, 17.4 mM NaCl, 0.21 mM KCl, 0.12 mM MgSO₄, 0.18 mM Ca(NO₃)₂, and 0.6 μM methylene blue) at 28° C., under a 14/10-h light/dark cycle.

### Mice

Male NMRI mice (weight 16-20 g) were acquired from Charles River Laboratories (France) and housed in polyacrylic cages under a 14/10-h light/dark cycle at 21° C. The animals were fed a pellet diet and water ad libitum and were allowed to acclimatize for one week before experimental procedures were conducted. Prior to the experiment, mice were isolated in polyacrylic cages with a pellet diet and water ad libitum for habituation overnight in the experimental room, to minimize stress.

### Example 4

### Tolerability Analysis in Zebrafish Larvae

Prior to behavioural and electrophysiological antiseizure analysis of compounds, their tolerability in zebrafish larvae was assessed at 10 and 2 μM by water immersion using 12 replicates per condition. After 20 (±2) h of exposure, the larvae were visually evaluated for signs of toxicity under a light microscope. Overall morphology, posture, touch response, oedema, signs of necrosis, swim bladder, and heartbeat were checked. A compound at a certain concentration was defined to be tolerated when no signs of toxicity were observed in comparison to VHC-treated larvae. When tolerance was observed at 10 μM, the tolerability of 50 μM was tested as well.

### Example 5

### Zebrafish Ethyl Ketopentanoate Seizure Model

### Behavioural Analysis

Experiments were performed as described in (16). In brief, a single 5 or 7 dpf larva was placed in each well of a 96-well plate and treated with either VHC (embryo medium with 1% DMSO) or test compound (dissolved in embryo medium, final solvent concentration of 1% DMSO) in a 100 μL volume. Larvae were incubated in the dark for 2 h at 28° C., whereafter 100 μL of either VHC (embryo medium with 1% DMSO) or 600 μM EKP (dissolved in embryo medium, final solvent concentration of 1% DMSO, 300 μM working concentration) was added to each well. Next, within 5 min the 96-well plate was placed in an automated tracking device (ZebraBox Viewpoint, France) and larval behaviour was video recorded for 30 min. The complete procedure was performed in dark conditions using infrared light. Total locomotor activity was recorded by ZebraLab software (Viewpoint, France) and expressed in actinteg units, which is the sum of pixel changes detected during the defined time interval (5 min). Larval behaviour was depicted as mean actinteg units per 5 min during the 30 min recording period and over consecutive time intervals. Data are pooled from independent experiments and expressed as mean±SD.

### Electrophysiology

Non-invasive LFP recordings were measured from the midbrain (optic tectum) of 7 dpf zebrafish larvae pre-incubated with VHC only, EKP only, or compound and EKP. Larvae were incubated for approximately 22 h with VHC (embryo medium with 1% DMSO) or test compound (dissolved in embryo medium, final solvent concentration of 1% DMSO) in a 100 μL volume. After incubation, VHC (embryo medium with 1% DMSO) or 600 μM EKP (dissolved in embryo medium, final solvent concentration of 1% DMSO, 300 μM working concentration) was added to the well for 15 min prior to recording. These steps occurred at 28° C., while further manipulation and electrophysiological recordings occurred at room temperature (±21° C.) and were performed as described before (16, 27). Each recording lasted 600 seconds.

A power spectral density (PSD) analysis of the recordings was performed using MatLab R2019 software (MATrix LABoratory, USA) as described in (26). The PSD estimate of each LFP recording was summed over each 10 Hz frequency band, ranging from 0 to 160 Hz. Next, the PSD estimates were normalized against the VHC control. Data are expressed as mean±SEM PSD per larva and per condition over the 20-90 Hz region. Outliers were identified via the ROUT test (Q=1%).

### Example 6

### Mouse 6-Hz (44 mA) Psychomotor Seizure Model

The antiseizure activity of compounds was investigated in the mouse 6-Hz (44 mA) psychomotor seizure model as described before (27). In brief, male NMRI mice (average body weight 30 g, range 23.5-38 g) were i.p. injected with 200 μL (injection volume was adjusted to the individual weight) of VHC (8% solutol/12% PEG200/80% water) or treatment (valproate or test compound dissolved in VHC) 30 min before seizure induction by corneal electrical stimulation (6 Hz, 0.2 ms rectangular pulse width, 3 s duration, 44 mA) using an ECT Unit 5780 (Ugo Basile, Comerio, Italy). Seizure behaviour was video recorded and seizure durations were determined by blinded video analysis by experienced researchers, familiar with the different seizure characteristics. Data are expressed as mean±SD.

### Example 7

### In Vitro ADME Profiling

In vitro ADME profiling of compounds was done by Eurofins Panlabs Inc (St Charles, MO, USA) using their ADME-Tox service (Cat Ref P375, Tier 1 ADME Panel). The following was tested: (1) aqueous solubility in PBS (pH 7.4), simulated intestinal fluid and simulated gastric fluid at 200 μM, (2) protein binding (plasma, human) at 10 μM, (3) A-B and B-A permeability (Caco-2, pH 6.5 and 7.4) at 10 μM, and (4) intrinsic clearance (liver microsomes, human) at 100 nM. Of note, the ADME-Tox service includes the determination of log D values, however, these could not be defined as the concentration of test compound in the aqueous buffer was below the limit of quantitation for both molecules. Hence, c Log P values were calculated based on the corresponding SMILES using Actelion's free OSIRIS DataWarrior software version 5.2.1. (28). The atom-based log P calculation method, called OsirisP, uses as an increment system and adds contributions of every atom based on its atom type. The prediction engine distinguishes a total of 368 atom types and was optimized using a training set of more than 5000 molecules with experimentally determined log P values. The free prediction engine has proven to outperform many alternative calculation methods (29).

### Aqueous Solubility

SGF, SIF and PBS buffers were prepared as follows: 34.2 mM NaCl, 84.7 mM HCl, 3.2 g/L pepsin (pH 2) for SGF, 50 mM KH₂PO₄, 38 mM NaOH, 10 g/L pancreatin (pH 7.5) for SIF and 137 mM NaCl, 2.7 mM KCl, 8.1 mM Na₂HPO₄ and 1.5 mM KH₂PO₄ (pH 7.4) for PBS. Test compounds were prepared at 200 μM concentrations in the corresponding buffer from a 10 mM stock solution (final solvent concentration of 2% DMSO). Buffer samples were mixed thoroughly followed by a 24 h incubation at room temperature. Samples were centrifuged and the supernatant was used for HPLC analysis. A calibration standard of the test compound was prepared at 200 μM in methanol/water (3:2 v/v) from the stock solution on the day of the analysis. Metoprolol, rifampicin, ketoconazole, phenytoin, haloperidol, simvastatin, diethylstilbestrol and tamoxifen were included in each assay. Aqueous solubility (μM) was determined by comparing peak areas.

### Plasma Protein Binding

Human plasma, used as the protein containing matrix, was spiked with the test compound at 10 μM (final solvent concentration of 1% DMSO). The assay was performed in a 96-well format in a dialysis block (Teflon) with the dialysate compartment containing PBS (pH 7.4) and the sample side containing an equal volume of the spiked protein matrix. The plate was incubated at 37° C. for 4 h. After incubation, samples were taken from both compartments, diluted with the phosphate buffer, followed by the addition of acetonitrile and centrifugation. The supernatants were then used for HPLC-MS/MS analysis. Acebutolol, quinidine and warfarin were tested in each assay. The percentage bound to proteins and the recovery were calculated as follows:

$$\begin{matrix}Protein binding ( \% ) = \frac{{Area}_{p} - {Area}_{b}}{{Area}_{p}} \times 100 \\ Recovery ( \% ) = \frac{{Area}_{p} + {Area}_{b}}{{Area}_{c}} \times 100\end{matrix}$$

Where Areaₚ, Area_{b} and Area_{c} are the peak area of the analyte in the protein matrix, the peak area of the analyte in the assay buffer and the peak area of the analyte in control sample, respectively.

### Caco-2 Permeability

Caco-2 cells were derived from a human colorectal adenocarcinoma. For permeability assays, cells were seeded at 1×105 cells/cm2 in 96 Multiscreen™ plates (Millipore) and used at days 21-25 post-seeding. Cells were used for 15 consecutive passages in culture. HBSS with 10 mM MES at pH 6.5 (apical side) or HBSS with HEPES at pH 7.4 (basolateral side) were used as the transport buffers. Test compounds were added at 10 μM concentration (final solvent concentration of 1% DMSO) to the apical side to determine apical to basolateral (A-B) transport and to the basal side to determine basolateral to apical (B-A) transport. For inhibition studies, 100 μM verapamil was included on both the A and B sides. Aliquots were taken from the donor side (A-B transport) at time zero and the end point, and from the receiver side (B-A transport) at the end point. Propranolol, labetalol, ranitidine, and colchicine (P-glycoprotein substrate) were included in each assay. Samples were analysed by HPLC-MS/MS for quantification. Pₐₚₚ (cm/sec) was calculated from the following equation:

$${P}_{app} = \frac{{V}_{R} \times {C}_{R , e n d}}{Δ t} \times \frac{1}{A \times ( {C}_{D , m i d} - {C}_{R , m i d} )}$$

Where V_{R} is the volume of the receiver, C is the concentration of the test compound (either at the donor or receiver side and at mid-point or end point of the incubation), Δt is the incubation time (seconds) and A is the surface area of the cell monolayer (0.11 cm²).

### Intrinsic Clearance

Metabolic stability of the test compounds was evaluated in pooled liver microsomes from human species. Test compounds were pre-incubated with liver microsomes in phosphate buffer (pH 7.4) in a 37° C. shaking water bath for 5 min and an NADPH-generating system was added to initiate the reaction. Samples were collected at time points of 0, 15, 30, 45 and 60 min and extracted with acetonitrile/methanol. After centrifugation, the supernatants were analysed by HPLC-MS/MS. T_{1/2} was calculated from the slope of the line obtained by plotting the natural logarithmic percentage (Ln %) of the test compound remaining in the reaction mixture vs. incubation time (min). CLᵢₙₜ (μL/min/mg protein) was calculated from T_{1/2} using following equation:

$$C {L}_{i n t} = \frac{0.693}{{T}_{1 / 2} \times protein conc .} \qquad Example 8. In vitro pharmacological profiling$$

In vitro pharmacological profiling of compounds was done against 78 functional assays for 47 common off-targets (30) by Eurofins DiscoverX Corporation (Fremont, CA, USA) using their SAFETYscan E/IC50 ELECT service (Cat Ref 87-1003DR, Safety47 Panel Dose Response). The following assays were performed: (1) GPCR cAMP modulation, (2) calcium mobilization, (3) nuclear hormone receptor assays, (4) KINOMEscan binding assays, (5) ion channel assays, (6) transporter assays and (7) enzymatic assays. Compounds were tested in a range of 10 concentrations, following a 3-fold serial dilution, starting from 10 μM. The assays were performed utilizing the PathHunter enzyme fragment complementation (EFC) technology, FLIPR®-based cellular screening assays, and KINOMEscan kinase binding assays.

GPCR cAMP Modulation

cAMP Hunter cell lines were expanded from freezer stocks. Prior to testing, cells were seeded in a total volume of 20 μL into white walled 384-well microplates and incubated at 37° C. cAMP modulation was determined using the DiscoverX HitHunter cAMP XS+ assay. For Gₛ agonist determination, cells were incubated with sample to induce response. For Gᵢ agonist determination, cells were incubated with sample in the presence of EC₈₀ forskolin to induce response. For both conditions, media was aspirated from cells and replaced with 15 μL 2:1 HBSS/10 mM HEPES:cAMP XS+ Ab reagent. Intermediate dilution of sample stocks was performed to generate 4× sample in assay buffer (containing 4×EC₈₀ forskolin in the case of antagonist determination). 5 μL of 4× sample was added to cells and incubated at 37° C. or room temperature (RT) for 30 or 60 min. Final assay vehicle concentration was 1%. For antagonist determination, cells were pre-incubated with sample followed by agonist challenge at the EC₈₀ concentration. Media was aspirated from cells and replaced with 10 μL 1:1 HBSS/HEPES:cAMP XS+ Ab reagent. 5 μL of 4× compound was added to the cells and incubated at 37° C. or RT for 30 min. 5 μL of 4×EC₈₀ agonist was added to the cells and incubated at 37° C. or RT for 30 or 60 min. For Gᵢ coupled GPCRs, EC₈₀ forskolin was included. After appropriate compound incubation, assay signal was generated through incubation with 20 μL cAMP XS+ ED/CL lysis cocktail for 1 h followed by incubation with 20 μL cAMP XS+ EA reagent for 3 h at RT. Microplates were read following signal generation with a PerkinElmer Envision™ instrument for chemiluminescent signal detection. Compound activity was analyzed using CBIS data analysis suite (ChemInnovation, CA). For Gₛ agonist mode assays, percentage activity was calculated using the following equation:

$$\% Activity = 100 \% \times \frac{mean RLU of test sample - mean RLU of vehicle control}{mean RLU of MAX control - mean RLU of vehicle control}$$

For Gₛ antagonist mode assays, percentage inhibition was calculated using the following equation:

$$\% Inhibition = 100 \% \times ( 1 - \frac{mean RLU of test sample - mean RLU of vehicle control}{mean RLU of {EC}_{80} control - mean RLU of vehicle control} )$$

For Gᵢ agonist mode assays, percentage activity was calculated using the following equation:

$$\% Activity = 100 \% \times ( 1 - \frac{mean RLU of test sample - mean RLU of MAX control}{mean RLU of vehicle control - mean RLU of MAX control} )$$

For Gᵢ antagonist or negative allosteric mode assays, percentage inhibition was calculated using the following equation:

$$\% Inhibition = 100 \% \times \frac{mean RLU of test sample - mean RLU of {EC}_{80} control}{mean RLU of forskolin positive control - mean RLU of {EC}_{80} control}$$

### Calcium Mobilization

Prior to testing, cells (10,000 cells/well) were expanded from freezer stocks and seeded into a total volume of 50 μL into black walled, clear-bottom, Poly-D-lysine coated 384-well microplates and incubated at 37° C. (≤0.2% DMSO concentration). Assays were performed in 1× dye loading buffer consisting of 1× Dye (DiscoverX, Calcium No Wash^{PLUS} kit, Catalog No. 90-0091), 1× Additive A and 2.5 mM Probenecid in HBSS/20 mM HEPES. Probenecid was prepared fresh. Prior to testing, media was aspirated from cells and replaced with 25 μL dye loading buffer. Cells were incubated for 45 min at 37° C. and then 20 min at RT. For agonist determination, 25 μL of 2× compound in HBSS/20 mM HEPES was added using a FLIPR Tetra (MDS). For antagonist determination, cells were pre-incubated with sample followed by agonist challenge at the EC₈₀ concentration. 25 μL of 2× sample was added and cells were incubated for 30 min at RT in the dark to equilibrate plate temperature. After incubation, antagonist determination was initiated by addition of 25 μL of 1× compound with 3×EC₈₀ agonist using FLIPR. For both agonist and antagonist formats, activity was measure on a FLIPR Tetra (MDS). Calcium mobilization was monitored for 2 min with a 5 sec baseline read.

FLIPR read—Area under the curve, was calculated for the entire 2 min read. Compound activity was analysed using CBIS data analysis suite (ChemInnovation, CA). For agonist mode assays, percentage activity was calculated using the following equation:

$$\% Activity = 100 \% \times \frac{mean RFU of test sample - mean RFU of vehicle control}{mean MAX RFU of control ligand - mean RFU of vehicle control}$$

For antagonist mode assays, percentage inhibition was calculated using the following equation:

$$\% Inhibition = 100 \% \times ( 1 - \frac{mean RFU of test sample - mean RFU of vehicle control}{mean RFU of {EC}_{80} control - mean RFU of vehicle control} )$$

### Nuclear Hormone Receptor Assays

Prior to testing, PathHunter NHR cell lines were expanded from freezer stocks and cells were seeded in a total volume of 20 μL into white walled 384-well microplates and incubated at 37° C. For agonist determination, cells were incubated with sample to induce response and an intermediate dilution of sample stocks was performed to generate 5× sample in assay buffer. 5 μL of 5× sample was added to the cells and incubated at 37° C. or RT for 3-16 h. Final assay vehicle concentration was 1%. For antagonist determination, cells were pre-incubated with antagonist followed by agonist challenge at the EC₈₀ concentration. Intermediate dilution of sample stocks was performed to generate 5× sample in assay buffer. 5 μL of 5× sample was added to the cells and incubated at 37° C. or RT for 60 min (vehicle concentration was 1%), followed by the addition of 5 μL of 6×EC₈₀ agonist in assay buffer and incubation at 37° C. or RT for 3-16 h. Assay signals were generated through a single addition of 12.5 or 15 μL (50% v/v) of PathHunter Detection reagent cocktail followed by a 1 h incubation at RT. Microplates were read following signal generation with a PerkinElmer Envision™ instrument for chemiluminescent signal detection. Compound activity was analysed using CBIS data analysis suite (ChemInnovation, CA). For agonist mode assays, percentage activity was calculated using the following equation:

$$\% Activity = 100 \% \times \frac{mean RLU of test sample - mean RLU of vehicle control}{mean MAX RLU of control ligand - mean RLU of vehicle control}$$

For antagonist mode assays, percentage inhibition was calculated using the following equation:

$$\% Inhibition = 100 \% \times ( 1 - \frac{mean RLU of test sample - mean RLU of vehicle control}{mean RLU of {EC}_{80} control - mean RLU of vehicle control} )$$

For select assays, the ligand response produced a decrease in receptor activity (inverse agonist with a constitutively active target). For those assays inverse agonist activity was calculated by the following equation:

$$\% Inverse Agonist Activity = 100 \% \times \frac{mean RLU of vehicle control - mean RLU of test sample}{mean RLU of vehicle control - mean MAX RLU of control ligand}$$

### KINOMEscan Binding Assays

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 min). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 min at RT to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads and test compounds in 1× loading buffer (20% SeaBlock, 0.17×PBS, 0.05% Tween 20, 6 mM DTT). All reactions were performed in polypropylene 384-well plates in a final volume of 0.02 mL. The assay plates were incubated at room temperature with shaking for 1 h and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at RT with shaking for 30 min. The kinase concentration in the eluates was measured by qPCR.

The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10 min hot start at 95° C., followed by 35 cycles of 95° C. for 15 sec, 60° C. for 1 min.

Percentage of response was calculated by the following equation:

$$\% Response = 100 \% \times ( \frac{test compound siganl - positive control signal}{negative compound signal - positive control signal} )$$

Percentage of control was converted to percentage of response using the following formula:

$$\% Response = 100 \% - \% Control$$

Binding constants (K_{d}s) were calculated with a standard dose-response curve using the Hill equation:

$$Response = Background + \frac{Signal - Background}{1 + \frac{{K}_{d}^{Hill Slope}}{{Dose}^{Hill Slope}}}$$

Where the Hill slope was set to −1 and curves were fitted using a non-linear least square fit with the Levenberg-Marquardt algorithm.

### Ion Channel Assays

Prior to testing, cell lines were expanded from freezer stocks and cells were seeded in a total volume of 20 μL into black walled, clear-bottom, Poly-D-lysine coated 384-well microplates and incubated at 37° C. As described in (2), assays were performed in 1× dye loading buffer consisting of 1× Dye and 2.5 mM Probenecid when applicable. Probenecid was prepared fresh. For agonist (opener) determination, cells were incubated with sample to induce response and an intermediate dilution of sample stocks was performed to generated 2-5× sample assay in buffer. 10-25 μL of 2-5× sample was added to the cells and incubated at 37° C. or RT for 30 min. Final assay vehicle concentration was 1%. For antagonist (blocker) determination, cells were pre-incubated with sample. Intermediate dilution of sample stocks was performed to generate 2-5× sample in assay buffer. After dye loading, cells were removed from the incubator and 10-25 μL of 2-5× sample was added to the cells in the presence of EC₈₀ agonist when appropriate. Cells were incubated for 30 min at RT in the dark to equilibrate the plate temperature. Vehicle concentration was 1%. Again, compound activity was measured as described earlier, on a FLIPR Tetra (MDS). Compound activity was analysed using CBIS data analysis suite (ChemInnovation, CA). For agonist mode assays, percentage activity was calculated using the following equation:

$$\% Activity = 100 \% \times \frac{mean RLU of test sample - mean RLU of vehicle control}{mean MAX control ligand - mean RLU of vehicle control}$$

For antagonist mode, percentage of inhibition was calculated as follows:

$$\% Inhibition = 100 \% \times ( 1 - \frac{mean RLU of test sample - mean RLU of vehicle control}{mean RLU of {EC}_{80} control - mean RLU of vehicle control} )$$

### Transporter Assays

Prior to testing, cell lines were expanded from freezer stocks and cells were seeded in a total volume of 25 μL into black walled, clear-bottom, Poly-D-lysine coated 384-well microplates and incubated at 37° C. After cell plating and incubation, media was removed an 25 μL of 1× compound in 1×HBSS/0.1% BSA was added. Compounds were incubated with cells for 30 min at 37° C. After compound incubation, 25 μL of 1× dye loading buffer (1× dye, 1×HBSS/20 mM HEPES) was added to the wells. Cells were incubated for 30-60 min at 37° C. After incubation, microplates were transferred to a PerkinElmer Envision™ instrument for fluorescent signal detection. Compound activity was analysed using CBIS data analysis suite (ChemInnovation, CA). For blocker mode assays, percentage of inhibition was calculated using the following formula:

$$\% Inhibition = 100 \% \times ( 1 - \frac{mean RLU of test sample - mean RLU of vehicle control}{mean RLU of positive control - mean RLU of vehicle control} )$$

### Enzymatic Assays

Enzyme preparations were sourced from various vendors-AChE (R&D Systems), COX1 and COX2 (BPS Bioscience), MAOA (Sigma), PDE3A and PDE4D2 (Signal Chem). AChE: enzyme and test compound were pre-incubated for 15 min at RT before substrate addition. Acetylthiocholine and DTNB were added and incubated at RT for 30 min. Signal was detected by measuring absorbance at 405 nm. COX1 and COX2: enzyme stocks were diluted in assay buffer (40 mM Tris-HCl, 1×PBS, 0.5 mM Phenol, 0.01% Tween 20 and 10 nM Hematin) and allowed to equilibrate with compounds at RT for 30 min (binding incubation). Arachidonic acid (1.7 μM) and Ampliflu Red (2.5 μM) were prepared and dispended into a reaction plate. Plates were read immediately on a fluorimeter with the emission detection at 590 nm and excitation wavelength 544 nm. MAOA: enzyme and test compound were pre-incubated for 15 min at 37° C. before substrate addition. The reaction was initiated by addition of kynuramine and incubated at 37° C. for 30 min. The reaction was terminated by addition of NaOH. The amount of 4-hydroquinoline formed was determined through spectrofluorometric readout with the emission detection at 380 nm and excitation wavelength 310 nm. PDE3A and PDE4D2: enzyme and test compound were pre-incubated for 15 min at RT before substrate addition. cAMP substrate (at a concentration equal to EC₈₀) was added and incubated at RT for 30 min. Enzyme reaction was terminated by addition of 9 mM IBMX. Signal was detected using the HitHunter® cAMP detection kit. Microplates were transferred to a PerkinElmer Envision™ instrument and read out as described for each assay. Compound activity was analysed using CBIS data analysis suite (ChemInnovation, CA). For enzyme activity assays, percentage inhibition was calculated using the following equation:

$$\% Inhibition = 100 \% \times ( 1 - \frac{mean RLU of test sample - mean RLU of vehicle control}{mean RLU of positive control - mean RLU of vehicle control} )$$

### Example 9

### Systematic Generation of a Compound Library of Propynones and Structural Derivatives

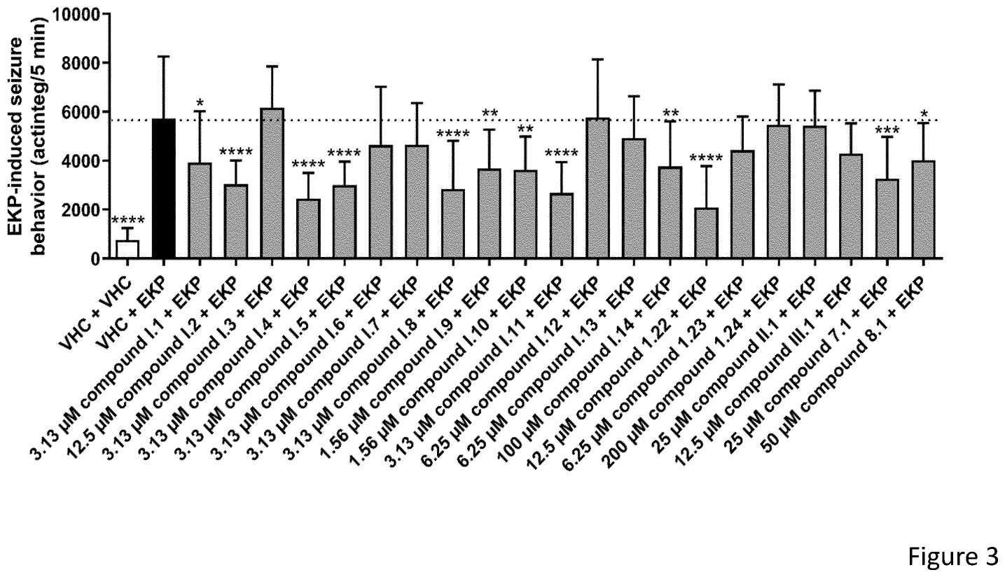

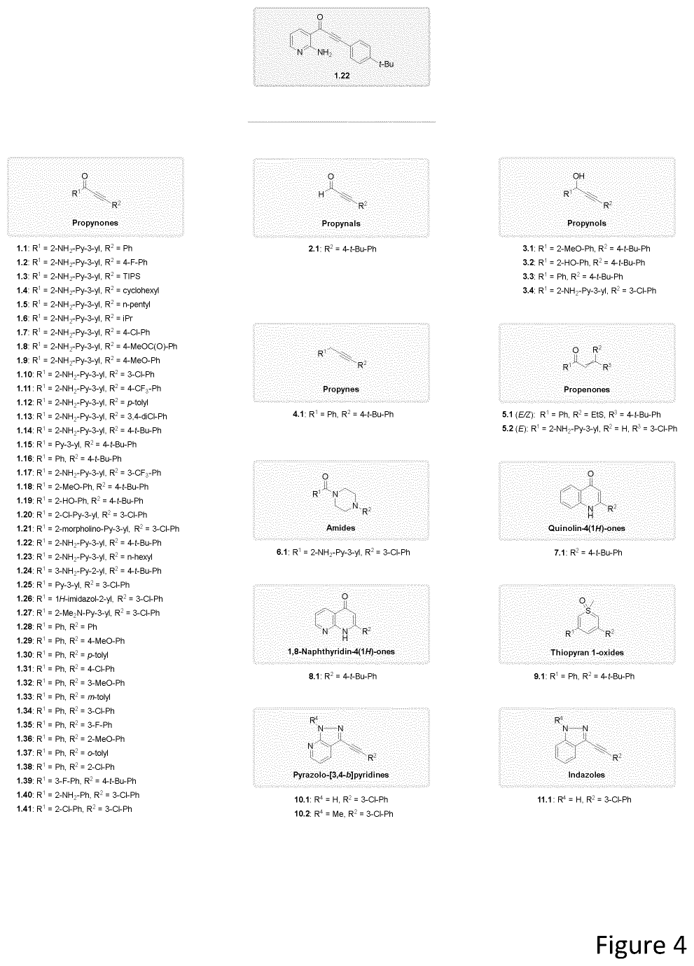

In our search for novel antiseizure compounds with the potential to treat drug-resistant seizures, the zebrafish EKP seizure model was used for hit identification and as critical gate-keeper for further investigation in the pharmacoresistant mouse 6-Hz (44 mA) psychomotor seizure model. An automated behavioural analysis was done of 7-day-old zebrafish larvae using video tracking (ViewPoint, France). In the course of our drug discovery process, several propynones (i.e., compounds I.1, I.2, I.4, I.5, I.8, I.9, I.10, I.11, I.14, and 1.22), a quinolinone (i.e., compound 7.1), and a naphthyridinone (i.e., compound 8.1) were observed to be active against EKP-induced drug-resistant seizures (FIGS. 2 and 3). Among them was compound 1-(2-aminopyridin-3-yl)-3-(4-(tert-butyl)phenyl)prop-2-yn-1-one (i.e., compound 1.22), a propynone, which was most effective and was validated for its antiseizure properties by electrophysiological analysis in zebrafish and by behavioural analysis in the mouse 6-Hz (44 mA) seizure model (data not shown). In addition, propynones I.6, I.7, and I.23, as well as quinolinone III.1, showed a non-significant reduction in EKP-induced drug-resistant seizures of approximately 20% (19-25%)(FIGS. 2 and 3).

To further investigate the antiseizure activities of propynones in general, and of compound 1.22 in particular, to improve our understanding of the structural necessities, and to select hits with an optimal safety-efficacy profile, a compound library of 56 structurally related small molecules was synthesized (FIG. 4). The library covers 11 compound classes (i.e., propynones, propynals, propynols, propynes, propenones, amides, quinolinones, naphthyridinones, thiopyranoxides, pyrazolopyridines, and indazoles) and includes more than 30 small molecules that are structurally novel and have been synthesized for the first time (examples 14-27). The library was generated in a systematic manner, designing each compound based on the efficacy of previously synthesized molecules in the behavioural assay.

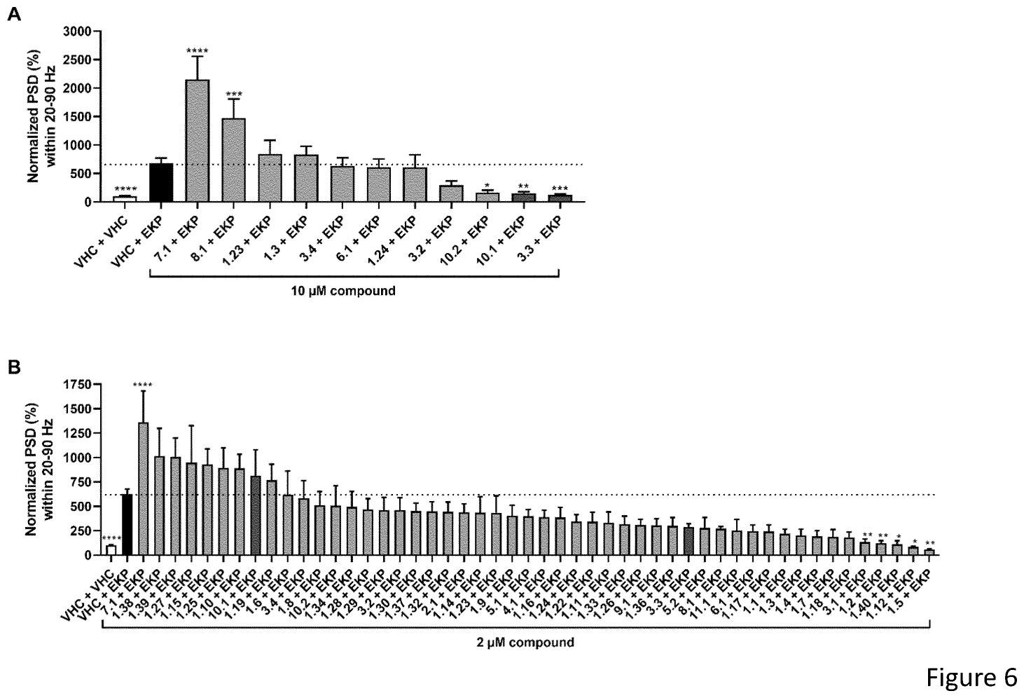

An overview of the behavioural antiseizure activity data is given in FIG. 5. The compounds were tested in 5-day-old zebrafish larvae at 2 and/or 10 μM, depending on their tolerability, after 2 h incubation time. Many significantly reduced EKP-induced seizure behaviour to various levels of efficacy. Among the antiseizure hits were compounds from 10 out of 11 classes tested, namely, propynones, propynals, propynols, propenones, amides, quinolinones, naphthyridinones, thiopyranoxides, pyrazolopyridines and indazoles. Hence, these data suggest that all are newly identified classes of antiseizure compounds. Of note, compound 4.1, a propyne, showed a non-significant reduction in EKP-induced seizure behaviour of approximately 23% and 19% at 2 and 10 μM, respectively.

### Example 10

### Electrophysiological Antiseizure Analysis in the Larval Zebrafish EKP Seizure Model

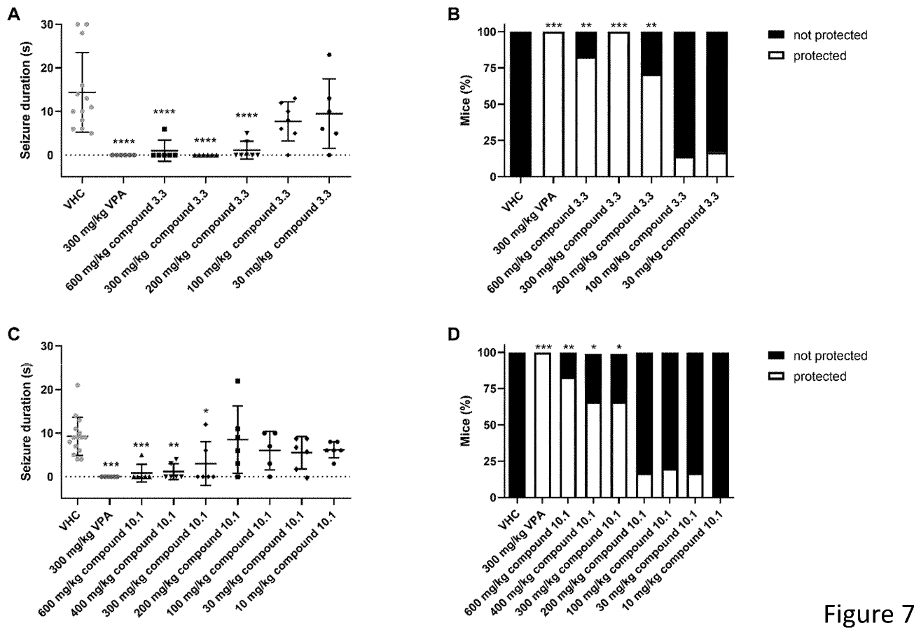

Given the challenge to differentiate between hits in terms of efficacy, all compounds underwent electrophysiological antiseizure analysis (FIG. 6 and Table 1). Based upon in-house experience with electrophysiological assessment of zebrafish larvae at different developmental stages, for the zebrafish EKP seizure model in particular, this analysis was performed on 7-dpf larvae. Moreover, a long-term treatment period of 22 h was chosen over a short-term of 2 h.

Prior to electrophysiological antiseizure analysis of the compounds, their tolerability in zebrafish larvae was assessed (Table 1). Compounds were administered to 6 dpf zebrafish larvae via water immersion at 10 and 2 μM for 20 (±2) h. At 7 dpf the larvae were visually evaluated for signs of toxicity under a light microscope. A compound was defined to be tolerated at the concentration used when no signs of toxicity were observed compared to VHC-treated larvae. When tolerance was observed at 2 and 10 μM, the tolerability of 50 μM was tested as well. All compounds were considered safe at 2 μM, except for compounds 1.13, 1.20, 1.21, 1.31, 1.35, and 1.41. These compounds are active and tolerated in the behavioural assay, which is performed on 5 dpf larvae and after 2 h (instead of 7 dpf, 22 h): 1.13, 1.20, 1.21, 1.31, and 1.35

An overview of the electrophysiological antiseizure activity data, also referred to as anti-epileptiform activity, is given in FIG. 6 and an overview of the tolerability of compounds and their efficacies against EKP-induced epileptiform discharges is given in table 1. Instead of visually quantifying the epileptiform events present in the LFP recordings, which has been the standard approach in zebrafish research so far (27, 31-33), power spectral density (PSD) computation of the LFP recordings was done (26), which is more rapid and not prone to subjectivity. This automated approach has been used before in the clinic and for rodent research (34-36). The methodology for PSD analysis of zebrafish LFP recordings was recently developed by the Laboratory for Molecular Biodiscovery (Prof. P. de Witte) and was observed to correlate well with visual analysis (26, 37). PSD analysis assumes that the epileptiform activity manifests as a high-power oscillation at certain frequencies (26). Thus, if epileptic activity occurs often during the recording an elevated PSD estimate should be found at the corresponding frequency band(s) (26). Indeed, EKP-treated control larvae show a significant higher PSD than VHC-treated control larvae (p≤0.0001). At 10 μM, compounds 3.3, 10.1, and 10.2, which were well tolerated (Table 1), show a significant decrease of the EKP-induced elevated PSD (p≤0.001, p≤0.01, and p≤0.05, respectively) (FIG. 6A). While compound 3.3 remained effective at 2 μM, compounds 10.1 and 10.2 did not (FIG. 6B). Many other compounds also (significantly) reduced EKP-induced seizure behaviour to various levels of efficacy (FIG. 6 and Table 1). An antiseizure hit was defined as a compound that had an electrophysiological antiseizure efficacy of at least 30%, which means that EKP-induced epileptiform activity (i.e., EKP-induced elevation in PSD) was lowered by at least 30%. Among the antiseizure hits (defined as at least 30% efficacy (see table 1)) were compounds from all classes except the quinolinones as compound 7.1 even significantly elevated the PSD (p≤0.0001 at 10 and 2 μM). Compound 4.1, a propyne, which did not show significant activity in the behavioural assay, was found to be effective as it (non-significantly) lowered the EKP-induced elevated PSD by 38% at 2 μM (FIG. 6B and Table 1). Taken together, the electrophysiological findings are in agreement with the behavioural data for the following compound classes: propynones, propynals, propynols, propenones, amides, naphthyridinones, thiopyranoxides, pyrazolopyridines, and indazoles, suggesting that these are indeed newly identified classes of antiseizure compounds. Moreover, also propynes are shown to be of interest given the antiepileptiform activity of compound 4.1 (FIG. 6B and Table 1). The same applies to the quinolinones as compound 7.1 did show significant antiseizure activity (p≤0.01 and p≤0.001) at the behavioural level (FIG. 5 and FIG. 3, respectively).

TABLE 1
Overview of compound tolerability and efficacy against EKP-induced
epileptiform discharges as measured by non-invasive LFP recordings
(i.e. electrophysiological antiseizure analysis).

| | tolerability | | | efficacy | |
|---|---|---|---|---|---|
| Compound | 50 μM | 10 μM | 2 μM | 10 μM | 2 μM |
| Compound 1.1 | ND | NT | T | ND | 65 |
| Compound 1.2 | ND | NT | T | ND | 80 |
| Compound 1.3 | NT | T | T | −23 | 67 |
| Compound 1.4 | ND | NT | T | ND | 69 |
| Compound 1.5 | ND | NT | T | ND | 91 |
| Compound 1.6 | ND | NT | T | ND | 1 |
| Compound 1.7 | ND | NT | T | ND | 70 |
| Compound 1.8 | ND | NT | T | ND | 18 |
| Compound 1.9 | ND | NT | T | ND | 35 |
| Compound 1.10 | ND | NT | T | ND | −42 |
| Compound 1.11 | ND | NT | T | ND | 47 |
| Compound 1.12 | ND | NT | T | ND | 86 |
| Compound 1.13 | ND | NT | NT | ND | ND |
| Compound 1.14 | ND | NT | T | ND | 60 |
| Compound 1.15 | ND | NT | T | ND | −49 |
| Compound 1.16 | ND | NT | T | ND | 38 |
| Compound 1.17 | ND | NT | T | ND | 61 |
| Compound 1.18 | ND | NT | T | ND | 71 |
| Compound 1.19 | ND | NT | T | ND | −23 |
| Compound 1.20 | ND | NT | NT | ND | ND |
| Compound 1.21 | ND | NT | NT | ND | ND |
| Compound 1.22 | ND | NT | T | ND | 45 |
| Compound 1.23 | NT | T | T | −25 | 31 |
| Compound 1.24 | T | T | T | 11 | 45 |
| Compound 1.25 | ND | NT | T | ND | −43 |
| Compound 1.26 | ND | NT | T | ND | 51 |
| Compound 1.27 | ND | NT | T | ND | −52 |
| Compound 1.28 | ND | NT | T | ND | 25 |
| Compound 1.29 | ND | NT | T | ND | 26 |
| Compound 1.30 | ND | NT | T | ND | 28 |
| Compound 1.31 | ND | NT | NT | ND | ND |
| Compound 1.32 | ND | NT | T | ND | 29 |
| Compound 1.33 | ND | NT | T | ND | 50 |
| Compound 1.34 | ND | NT | T | ND | 21 |
| Compound 1.35 | ND | NT | NT | ND | ND |
| Compound 1.36 | ND | NT | T | ND | 52 |
| Compound 1.37 | ND | NT | T | ND | 28 |
| Compound 1.38 | ND | NT | T | ND | −63 |
| Compound 1.39 | ND | NT | T | ND | −61 |
| Compound 1.40 | ND | NT | T | ND | 82 |
| Compound 1.41 | ND | NT | NT | ND | ND |
| Compound 2.1 | ND | NT | T | ND | 30 |
| Compound 3.1 | ND | NT | T | ND | 79 |
| Compound 3.2 | T | T | T | 57 | 26 |
| Compound 3.3 | NT | T | T | 82 | 54 |
| Compound 3.4 | NT | T | T | 7 | 7 |
| Compound 4.1 | ND | NT | T | ND | 38 |
| Compound 5.1 | ND | NT | T | ND | 36 |
| Compound 5.2 | ND | NT | T | ND | 55 |
| Compound 6.1 | T | T | T | 10 | 61 |
| Compound 7.1 | T | T | T | −217 | −118 |
| Compound 8.1 | T | T | T | −117 | 57 |
| Compound 9.1 | ND | NT | T | ND | 51 |
| Compound 10.1 | T | T | T | 77 | −30 |
| Compound 10.2 | T | T | T | 76 | 19 |
| Compound 11.1 | ND | NT | T | ND | 60 |

Legend:
T Tolerated
NT Not Tolerated
ND Non Determined
Left column: compound IDs of the synthesized propynones, propynals, propynols, propynes, propenones, amides, quinolinones, naphthyridinones, thiopyranoxides, and pyrazolopyridines.
Middle column: compound tolerability at 2, 10, and 50 μM.
Right column: mean compound efficacy (normalized data) against EKP-induced epileptiform discharges, as measured by non-invasive LFP recordings (i.e. electrophysiological antiseizure analysis), at 2 and 10 μM.

Although many compounds show potential against EKP-induced drug-resistant seizures in the zebrafish model, compounds 3.3, 10.1, and 10.2 show the most optimal tolerability-efficacy profile. Compounds 3.3 and 10.1 were selected for further investigation in terms of safety (i.e., in vitro pharmacological profiling for 47 common off-targets) and efficacy (i.e., behavioural antiseizure analysis in the mouse 6-Hz (44 mA) psychomotor seizure model, in vitro ADME profiling, and pharmacokinetic analysis in naïve mice). In addition, given the chemical diversity present among the numerous antiseizure hits, compounds 6.1 and 8.1 were also selected for behavioural antiseizure analysis in the mouse 6-Hz (44 mA) psychomotor seizure model. This to investigate whether the activity of these four new classes of antiseizure compounds in the zebrafish EKP model of drug-resistant seizures translates to a standard mouse seizure model of drug-resistant seizures. Selection criteria included heterocycles versus carbocycles, monocyclic compounds versus bicyclic compounds, a diverse set of functional groups, and—feasibly—distinctive pharmacological profiles.

### Example 11

### Validation of Antiseizure Activity of Compounds 3.3, 6.1, 8.1, and 10.1 in the Mouse 6-Hz (44 mA) Psychomotor Seizure Model

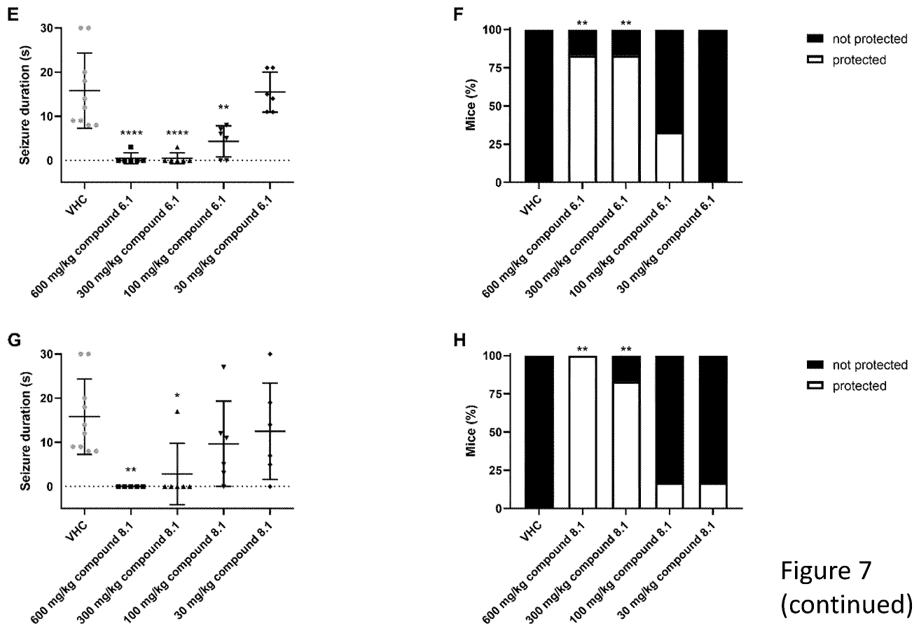

We aimed to investigate whether the anti-epileptiform activity of compounds 3.3, 6.1, 8.1, and 10.1 observed in the zebrafish EKP model, translates to a standard mouse seizure model of drug-resistant seizures. Among the rodent seizure models available, the mouse 6-Hz (44 mA) model was selected. The 6-Hz (44 mA) mouse model is a gold standard in current antiseizure drug discovery that can detect compounds with novel antiseizure mechanisms and with potential activity against pharmacoresistant seizures (12, 38, 39). The 6-Hz 44 mA model is an acute model of pharmacoresistant focal impaired awareness seizures, previously referred to as complex partial or psychomotor seizures that are induced by a low frequency, long duration corneal electrical stimulation (6 Hz, 0.2 ms rectangular pulse width, 3 s duration, 44 mA). Seizures are typically characterized by a clonic phase and stereotypical automatic behaviours like stun, forelimb clonus, Straub tail and vibrissae twitching. For the experiments with compounds 3.3 and 10.1 (FIG. 7A-D), VHC injected mice showed characteristic seizure behaviour with a mean (±SD) duration of 14.4 s (±9.1 s) and 9.3 s (±4.4 s) (FIGS. 7A and C). In both experiments, 300 mg/kg valproate was used as a positive control and completely protected mice against the electrically induced seizures (p<0.0001 and p<0.001), as expected (12, 31) (FIGS. 7A and C). For the experiments with compounds 6.1 and 8.1 (FIG. 7E-H), VHC injected mice showed characteristic seizure behaviour with a mean (±SD) duration of 15.8 s (±8.5 s) (FIGS. 7E and G).

Mice that were injected with compound 3.3 displayed a dose-response relationship (FIG. 7A-B), with nearly to full protection at the highest doses of 600 mg/kg (p<0.0001, mean duration of 1.00 s (±2.5 s)), 300 mg/kg (p<0.0001, mean duration of 0 s (±0 s)) and 200 mg/kg (p<0.0001, mean duration of 1.1 s (±2.0 s)), but not at lower doses of 100 mg/kg (mean duration of 7.7 s (±4.5 s)) and 30 mg/kg (mean duration of 9.5 s (±8.0 s)) (FIG. 7A). Strikingly, one in six mice injected with the highest dose of compound 3.3 (600 mg/kg), was not fully protected in comparison to the lower dose of 300 mg/kg, where all mice were fully protected. Interestingly, this mouse had a low body weight of only 23.5 g vs. 30 g on average (body weight range: 23.5±38 g).

Mice that were injected with compound 10.1 displayed a dose-response relationship (FIG. 7C-D), with nearly full protection at the highest doses of 600 mg/kg (p=0.0007, mean duration of 0.8 s (±2.0 s)), 400 mg/kg (p=0.0011, mean duration of 1.2 s (±1.8 s)), 300 mg/kg (p=0.0171, mean duration of 3.0 s (±5.0 s)), and little to no protection at the lower doses of 200 mg/kg (mean duration of 8.5 s (±7.7 s)), 100 mg/kg (mean duration of 6.0 s (±4.4)), 30 mg/kg (mean duration of 5.5 s (±3.7 s)), and 10 mg/kg (mean duration of 6.2 s (±1.8 s)) (FIG. 7C).

Mice that were injected with compound 6.1 displayed a dose-response relationship (FIG. 7E-F), with nearly protection at the highest doses of 600 mg/kg (p=0.0004, mean duration of 0.5 s (±1.2 s)), 300 mg/kg (p=0.0004, mean duration 0.5 s (±1.2 s)) and 100 mg/kg (p=0.0123, mean duration of 4.3 s (±3.5 s)), but not at a lower dose of 30 mg/kg (mean duration of 15.5 s (±4.6 s)) (FIG. 7E).

Mice that were injected with compound 8.1 displayed a dose-response relationship (FIG. 7G-H), with full protection at the highest dose of 600 mg/kg (p=0.0006, mean duration of 0 s (±0 s)), and little to no protection at lower doses of 300 mg/kg (p=0.0034, mean duration of 2.8 s (±6.9 s)), 100 mg/kg (mean duration of 9.7 s (±9.7 s)), and 30 mg/kg (mean duration of 12.5 (±10.9 s)) (FIG. 7G).

We can conclude that the anti-epileptiform activity that was observed in the larval EKP-model translates to a standard mouse model of pharmacoresistant seizures. This demonstrates the effectiveness of the zebrafish-based antiseizure drug discovery approach and the potential of the investigated compounds.

### Example 12

### In Vitro ADME Profiling of Compounds 3.3 and 10.1

To characterize the absorption, distribution, metabolism and excretion (ADME) properties of compounds 3.3 and 10.1, in vitro ADME profiling was performed by Eurofins Panlabs Inc (St Charles, MO, USA) using their ADME-Tox service (Cat Ref P375, Tier 1 ADME Panel) (Table 2). The Log D values could not be defined as the concentration of test compound in the aqueous buffer was below the limit of quantitation for both molecules. Hence, c Log P values were calculated based on the corresponding SMILES using Actelion's free OSIRIS DataWarrior software version 5.2.1. (28) (Table 2). Compound 3.3 displayed a c Log P value of 4.635, which points out a low hydrophilicity and high lipophilicity. Compound 10.1, on the other hand, showed a lower c Log P value of 2.996, and is thus less lipophilic. The solution properties showed high plasma protein binding for both compound 3.3 and 10.1 of 99.8% and 99.65%, respectively, and an acceptable solubility. Solubility studies were performed in PBS (pH 7.4), simulated intestinal fluid (pH 7.5), and simulated gastric fluid (pH 2.0). Compound 3.3 showed a solubility of 17.7 μM in PBS, 102.5 μM in simulated intestinal fluid, and 24.5 μM in simulated gastric fluid. Compound 10.1 had a lower solubility in PBS of <0.1 μM and in simulated intestinal fluid of 26.7 μM. In simulated gastric fluid compound 10.1 had a higher solubility of 26.7 μM. In vitro absorption studies in Caco-cells showed for compound 3.3 a transport activity from the apical to basolateral direction of 0.4×10⁻⁶ cm/s and from the basolateral to apical direction of 0.1×10⁻⁶ cm/s. For compound 10.1, a transport activity from the apical to basolateral direction of 3.3×10⁻⁶ cm/s and from basolateral to apical direction of 0.7×10⁻⁶ cm/s was observed. A percentual recovery of 6 and 13% from the apical to basolateral direction and of 15 and 44% from basolateral to apical direction was observed for compounds 3.3 and 10.1, respectively. Overall, the permeability is rather low for both compounds. Finally, in vitro metabolism studies were done to determine the intrinsic clearance in human liver microsomes and the half-life. A half-life of 19 min was observed for compound 3.3 who showed an intrinsic clearance of 380.6 μL/min/mg, and a half-life of 10 min was observed for compound 10.1 who showed an intrinsic clearance of 748.5 μL/min/mg. Taken together, both compounds show an acceptable ADME profile, which could be improved in a hit-to-lead optimization process.

TABLE 2
In vitro ADME profiles of compounds 3.3 and 10.1 performed by Eurofins Panlabs
Inc using their ADME-Tox service (Cat Ref P375, Tier 1 ADME Panel).

| | | | | | | In vitro | | | |
|---|---|---|---|---|---|---|---|---|---|
| | Solution properties | | | | | absorption | | In vitro | |
| | | PBS | Simulated | Simulated | Plasma | A-B | B-A | metabolism | |
| | | (pH | intestinal | gastric | protein | permeability | permeability | | Intrinsic |
| | | 7.4, | fluid | fluid | binding | (10⁻⁶ | (10⁻⁶ | T_{1/2} | clearance |
| | cLogP | μM) | (μM) | (μM) | (%) | cm/s) | cm/s) | (min) | (μL/min/mg) |
| Compound 3.3 | 4.635 | 17.7 | 102.5 | 24.5 | 99.8 | 0.4 | 0.1 | 19 | 380.6 |
| Compound 10.1 | 2.996 | <0.1 | 26.7 | 26.7 | 99.65 | 3.3 | 0.7 | 10 | 748.5 |

Solution properties like aqueous solubility in PBS (pH 7.4), simulated intestinal fluid (pH 7.5) and simulated gastric fluid (pH 2) at 200 μM and plasma protein binding (human) at 10 μM were evaluated.
In vitro studies like A-B and B-A permeability (Caco-2, pH 6.5 and 7.4) at 10 μM, and in vitro metabolism studies like intrinsic clearance (human liver microsomes) at 100 nM were performed.
cLogP values were calculated based on the corresponding SMILES using Data Warrior version 5.2.1.

### Example 13

### In Vitro Pharmacological Profiling of Compounds 3.3 and 10.1

To identify potential off-target effects of compounds 3.3 and 10.1 in vitro pharmacological profiling was performed by Eurofins Panlabs using their Safety47 Screen. The in vitro pharmacological profiles are shown in table 3, showing the IC50 and EC50 values, as well as the maximal response. For compound 3.3, the following IC50 values were obtained: 4.3 μM for the muscarinic acetylcholine receptor M₂ (CHRM2), 7.9 μM for the dopamine receptor D₂ (DRD2S), 6.4 μM for the 5-hydroxytryptamine 1B (HTR1B) receptor, and 6.9 μM for the alpha-4 beta-2 nicotinic acetylcholine receptors. For compound 10.1, on the other hand, no IC50 or EC50 values were observed at the highest test concentration of 10 μM. Thus, while compound 3.3 shows 4 potential off-targets compound 10.1 has none. Of note, the potencies of compound 3.3 are low with IC50 values of 4-8 μM, which is typically considered safe. Nonetheless, these findings should be further investigated.

TABLE 3
In vitro pharmacological profiles of compounds 3.3 and 10.1.

| Assay description | | | | Compound 3.3 | | | Compound 10.1 | | |
|---|---|---|---|---|---|---|---|---|---|
| Assay | Assay | | Result | Value | RC50 | Max | Value | RC50 | Max |
| name | target | Mode | type | prefix | (μM) | response | prefix | (μM) | response |
| Calcium Flux | ADORA2A | Agonist | EC50 | > | 10 | 1.8 | > | 10 | 3.0 |
| Calcium Flux | ADRA1A | Agonist | EC50 | > | 10 | 0.6 | > | 10 | 0.6 |
| Calcium Flux | AVPR1A | Agonist | EC50 | > | 10 | 8.7 | > | 10 | 7.9 |
| Calcium Flux | CCKAR | Agonist | EC50 | > | 10 | 0.2 | > | 10 | 0.3 |
| Calcium Flux | CHRM1 | Agonist | EC50 | > | 10 | 1.8 | > | 10 | 1.8 |
| Calcium Flux | CHRM3 | Agonist | EC50 | > | 10 | 0 | > | 10 | 2.2 |
| Calcium Flux | EDNRA | Agonist | EC50 | > | 10 | 0 | > | 10 | 1.8 |
| Calcium Flux | HRH1 | Agonist | EC50 | > | 10 | 0 | > | 10 | 0 |
| Calcium Flux | HTR2A | Agonist | EC50 | > | 10 | 0 | > | 10 | 0 |
| Calcium Flux | HTR2B | Agonist | EC50 | > | 10 | 0 | > | 10 | 0 |
| Calcium Flux | ADORA2A | Antagonist | IC50 | > | 10 | 0 | > | 10 | 0 |
| Calcium Flux | ADRA1A | Antagonist | IC50 | > | 10 | 0 | > | 10 | 0 |
| Calcium Flux | AVPR1A | Antagonist | IC50 | > | 10 | 0 | > | 10 | 0 |
| Calcium Flux | CCKAR | Antagonist | IC50 | > | 10 | 0 | > | 10 | 0 |
| Calcium Flux | CHRM1 | Antagonist | IC50 | > | 10 | 0 | > | 10 | 5.5 |
| Calcium Flux | CHRM3 | Antagonist | IC50 | > | 10 | 0 | > | 10 | 0 |
| Calcium Flux | EDNRA | Antagonist | IC50 | > | 10 | 0 | > | 10 | 0 |
| Calcium Flux | HRH1 | Antagonist | IC50 | > | 10 | 0 | > | 10 | 1.0 |
| Calcium Flux | HTR2A | Antagonist | IC50 | > | 10 | 0 | > | 10 | 0 |
| Calcium Flux | HTR2B | Antagonist | IC50 | > | 10 | 0 | > | 10 | 8.8 |
| cAMP | ADRA2A | Agonist | EC50 | > | 10 | 0 | > | 10 | 20.7 |
| cAMP | ADRB1 | Agonist | EC50 | > | 10 | 0.6 | > | 10 | 0 |
| cAMP | ADRB2 | Agonist | EC50 | > | 10 | 1.9 | > | 10 | 0.3 |
| cAMP | CHRM2 | Agonist | EC50 | > | 10 | 0 | > | 10 | 32.5 |
| cAMP | CNR1 | Agonist | EC50 | > | 10 | 9.8 | > | 10 | 20.1 |
| cAMP | CNR2 | Agonist | EC50 | > | 10 | 38.1 | > | 10 | 49.3 |
| cAMP | DRD1 | Agonist | EC50 | > | 10 | 1.2 | > | 10 | 1.3 |
| cAMP | DRD2S | Agonist | EC50 | > | 10 | 0 | > | 10 | 15.0 |
| cAMP | HRH2 | Agonist | EC50 | > | 10 | 2.2 | > | 10 | 0.4 |
| cAMP | HTR1A | Agonist | EC50 | > | 10 | 0 | > | 10 | 9.1 |
| cAMP | HTR1B | Agonist | EC50 | > | 10 | 5.2 | > | 10 | 9.3 |
| cAMP | OPRD1 | Agonist | EC50 | > | 10 | 3.6 | > | 10 | 12.1 |
| cAMP | OPRK1 | Agonist | EC50 | > | 10 | 0 | > | 10 | 5.4 |
| cAMP | OPRM1 | Agonist | EC50 | > | 10 | 0 | > | 10 | 9.9 |
| cAMP | ADRA2A | Antagonist | IC50 | > | 10 | 13.0 | > | 10 | 0 |
| cAMP | ADRB1 | Antagonist | IC50 | > | 10 | 2.2 | > | 10 | 40.3 |
| cAMP | ADRB2 | Antagonist | IC50 | > | 10 | 0 | > | 10 | 39.9 |
| cAMP | CHRM2 | Antagonist | IC50 | = | 4.3 | 90.1 | > | 10 | 0 |
| cAMP | CNR1 | Antagonist | IC50 | > | 10 | 17.5 | > | 10 | 0 |
| cAMP | CNR2 | Antagonist | IC50 | > | 10 | 38.3 | > | 10 | 0 |
| cAMP | DRD1 | Antagonist | IC50 | > | 10 | 2.7 | > | 10 | 17.2 |
| cAMP | DRD2S | Antagonist | IC50 | = | 7.9 | 63.5 | > | 10 | 0 |
| cAMP | HRH2 | Antagonist | IC50 | > | 10 | 2.7 | > | 10 | 13.9 |
| cAMP | HTR1A | Antagonist | IC50 | > | 10 | 47.2 | > | 10 | 0 |
| cAMP | HTR1B | Antagonist | IC50 | = | 6.4 | 66.2 | > | 10 | 0 |
| cAMP | OPRD1 | Antagonist | IC50 | > | 10 | 5.9 | > | 10 | 0 |
| cAMP | OPRK1 | Antagonist | IC50 | > | 10 | 8.9 | > | 10 | 0 |
| cAMP | OPRM1 | Antagonist | IC50 | > | 10 | 0.8 | > | 10 | 0 |
| Ion Channel | CAV1.2 | Blocker | IC50 | > | 10 | 7.2 | > | 10 | 0 |
| Ion Channel | GABAA | Blocker | IC50 | > | 10 | 8.7 | > | 10 | 5.2 |
| Ion Channel | hERG | Blocker | IC50 | > | 10 | 3.8 | > | 10 | 6.6 |
| Ion Channel | HTR3A | Blocker | IC50 | > | 10 | 50.1 | > | 10 | 19.7 |
| Ion Channel | KvLQT1/minK | Blocker | IC50 | > | 10 | 47.5 | > | 10 | 0 |
| Ion Channel | nAChR (a4/b2) | Blocker | IC50 | = | 6.9 | 73.9 | > | 10 | 1.5 |
| Ion Channel | NAV1.5 | Blocker | IC50 | > | 10 | 59.9 | > | 10 | 21.5 |
| Ion Channel | NMDAR | Blocker | IC50 | > | 10 | 0 | > | 10 | 0 |
| | (1A/2B) | | | | | | | | |
| Ion Channel | GABAA | Opener | EC50 | > | 10 | 0 | > | 10 | 1.3 |
| Ion Channel | HTR3A | Opener | EC50 | > | 10 | 0 | > | 10 | 3.4 |
| Ion Channel | KvLQT1/minK | Opener | EC50 | > | 10 | 0 | > | 10 | 2.0 |
| Ion Channel | nAChR | Opener | EC50 | > | 10 | 0 | > | 10 | 0 |
| | (a4/b2) | | | | | | | | |
| Ion Channel | NMDAR | Opener | EC50 | > | 10 | 0 | > | 10 | 0 |
| | (1A/2B) | | | | | | | | |
| Binding | INSR | Inhibitor | IC50 | > | 10 | 10.3 | > | 10 | 0 |
| Binding | LCK | Inhibitor | IC50 | > | 10 | 0 | > | 10 | 0 |
| Binding | ROCK1 | Inhibitor | IC50 | > | 10 | 0 | > | 10 | 4.2 |
| Binding | VEGFR2 | Inhibitor | IC50 | > | 10 | 0 | > | 10 | 5.4 |
| NHR Nuclear | AR | Agonist | EC50 | > | 10 | 1.4 | > | 10 | 0 |
| Translocation | | | | | | | | | |
| NHR Nuclear | AR | Antagonist | IC50 | > | 10 | 17.1 | > | 10 | 4.6 |
| Translocation | | | | | | | | | |
| NHR Protein | GR | Agonist | EC50 | > | 10 | 1.2 | > | 10 | 0 |
| Interaction | | | | | | | | | |
| NHR Protein | GR | Antagonist | IC50 | > | 10 | 0 | > | 10 | 2.6 |
| Interaction | | | | | | | | | |
| Enzymatic | AChE | Inhibitor | IC50 | > | 10 | 3.8 | > | 10 | 1.3 |
| Enzymatic | COX1 | Inhibitor | IC50 | > | 10 | 9.9 | > | 10 | 0 |
| Enzymatic | COX2 | Inhibitor | IC50 | > | 10 | 0 | > | 10 | 0 |
| Enzymatic | MAOA | Inhibitor | IC50 | > | 10 | 4.4 | > | 10 | 12.4 |
| Enzymatic | PDE3A | Inhibitor | IC50 | > | 10 | 0.6 | > | 10 | 0.1 |
| Enzymatic | PDE4D2 | Inhibitor | IC50 | > | 10 | 0 | > | 10 | 2.7 |
| Transporter | DAT | Blocker | IC50 | > | 10 | 0 | > | 10 | 33.1 |
| Transporter | NET | Blocker | IC50 | > | 10 | 0 | > | 10 | 13.6 |
| Transporter | SERT | Blocker | IC50 | > | 10 | 0 | > | 10 | 0 |

### Example 14

### Synthesis of the Compounds

### Reagents & Materials

All reagents were obtained from commercially available sources (Sigma-Aldrich, Acros Organics, J&K Scientific, AK Scientific, Fisher Scientific, Manchester Organics, Fluorochem, Janssen Chimica and Iris Biotech) and were used without any further purification, unless stated otherwise. 3-bromopyridin-2-amine purchased from Acros was purified via column chromatography on silica gel prior to use. Dry triethylamine used in the carbonylative Sonogashira reactions was previously distilled over sodium or CaH₂ and stored under argon atmosphere. Dry DCM and dry dioxane were purchased via Acros Organics in 500 mL glass bottles equipped with an AcroSeal® and were stabilized with amylene (approximately 50 ppm) and BHT (2-5 ppm), respectively, and stored over molecular sieves. Dry THF (unstabilized) and dry toluene were bought via Sigma-Aldrich in 18 L steel drums and were dispensed using a MBRAUN MB-SPS-800 Solvent Purification System.

Unless stated otherwise, all reactions were performed under N₂ or Ar atmosphere and stirred magnetically with PTFE-coated magnetic stirring bars at 350-450 rpm. With the exception of the EKP synthesis (see p. S54), solvents were evaporated under reduced pressure using a rotary evaporator at a bath temperature of 50° C. Final compounds were dried under high vacuum (10⁻³ mbar) at room temperature. Yields refer to isolated compounds after purification with a purity ≥99.8 mol % based on ¹H NMR analysis.

### NMR

¹H and ¹³C nuclear magnetic resonance (NMR) spectra were recorded on a Bruker Avance 300 spectrometer with a Bruker 300 UltraShield™ magnet system (operating at a ¹H basic frequency of 300.13 MHz), a Bruker Avance III HD 400 spectrometer with a Bruker Ascend™ 400 magnet system (operating at a ¹H basic frequency of 400.17 MHz) or a Bruker Avance II+ 600 spectrometer with a Bruker 600 UltraShield™ magnet system (operating at a ¹H basic frequency of 600.13 MHz) in chloroform-d (CDCl₃) or DMSO-d₆. The data were recorded at room temperature using Bruker TopSpin 3.6.1 and processed and analyzed using Bruker TopSpin 4.1.1. The δ-values are expressed in parts per million (ppm). ¹H data were calibrated using tetramethylsilane (TMS) as an internal reference, while ¹³C data were calibrated using the deuterated solvents as internal reference (for CDCl₃ a 1:1:1 triplet at 77.16 ppm and for DMSO-d₆ a 1:3:6:7:6:3:1 septet at 39.52 ppm). The following acronyms were used: s (singlet), d (doublet), t (triplet), q (quartet), quint (quintet), sext (sextet), sept (septet), ABq (AB quartet), m (multiplet), br (broadened). The prefix app. denotes the apparent multiplicity of a signal.

### TLC

Thin layer chromatography (TLC) analysis was performed using Sigma-Aldrich 20×20 cm precoated, glass TLC plates with fluorescent indicator at 254 nm (article number 99571: layer thickness 250 μm, particle size 8.0-12.0 μm, average pore diameter 60 Å). Visualization of the products was achieved by UV-radiation at 254 nm or using a staining solution of saturated o-dianisidine in glacial acetic acid.

### Flash Column Chromatography

Flash column chromatography (medium pressure liquid chromatography, MPLC) was performed using a Büchi Sepacore® flash system, consisting of a Büchi C-660 Fraction Collector, a Büchi C-615 Pump Manager controlling two Büchi C-605 Pump Modules, a Knauer WellChrom K-2501 spectrophotometer (operating at 254 nm), and a Linseis D120S plotter. Büchi PP cartridges (40/150 mm) were filled with 90 g of Acros ultra-pure silica gel for column chromatography (article number 360050300, particle size 40-60 μm, average pore diameter 60 Å) using a Büchi C-670 Cartridger. Unless stated otherwise, the eluent flow rate was set to 25 mL/min.

### Column Chromatography

Column chromatography was performed using Acros silica gel for chromatography article number 240370300, particle size 0.060-0.200 mm, average pore diameter 60 Å).

### Microwave

Microwave-assisted reactions were performed using a single-mode CEM Discover® LabMate operating at 2.465 GHz. The reaction mixture was magnetically stirred and continuously irradiated at a power of 0 to 300 W using the standard absorbance level of 100 W. The reactions were carried out in 10 mL glass microwave vials, sealed with a snap-on cap with septum. When the reaction was finished, the vial was cooled down to ambient temperature under a stream of compressed air. The reaction parameters (temperature, pressure, output power and reaction time) were monitored by a computer using Synergy 1.39 software.

### HR-MS

High-resolution mass spectra (HR-MS) were acquired on a quadrupole orthogonal acceleration time-of-flight mass spectrometer (Synapt G2 HDMS, Waters, Milford, MA). Samples were infused at 3 μL/min and spectra were obtained in positive or negative ionization mode with a resolution of 15000 (FWHM) using leucine enkephalin as lock mass.

HR-MS data were acquired for all new compounds and a small number of known compounds for which no HR-MS data were available in literature.

### LR-MS

Low resolution mass spectra (LR-MS) were recorded using an Agilent 1100 HPLC system, consisting of a G1311A quaternary pump and solvent module, a G1313A automatic liquid sampler (ALS), a G1315A diode-array detector (DAD, operating at 215, 254, 280, 320 and 365 nm) and a G1316A thermostated column compartment (TCC, kept at a constant temperature of 25° C.) without an HPLC column (direct injection method). The HPLC system was coupled to an Agilent 6110 single-quadrupole mass spectrometer with an electrospray ionization (ESI) source (capillary voltage 3500 V), operating in the positive mode. Samples were prepared by dissolving the compound in methanol to an approximate concentration of 1 mM. Each sample was automatically injected onto the HPLC system (injection volume 10 μL) and run isocratically in 100% methanol (LC-MS grade, Fisher Scientific) with a flow rate of 0.2 mL/min. Data were acquired using Agilent LC/MSD ChemStation software rev. B.04.03-SP2 [105] and processed and analyzed using ACD/Spectrus Processor 2019.1.2.

LR-MS data were acquired for all compounds for which HR-MS data were already available in literature.

### ATR-FT-IR

Attenuated total reflection (ATR) Fourier-transformed infrared (FT-IR) spectra were recorded on a Bruker Alpha-P FT-IR spectrometer with single reflection Platinum ATR accessory. Samples were analyzed neat in solid or liquid state without any further manipulations. The data were recorded at room temperature using Bruker OPUS 7.5 and processed and analyzed using ACD/Spectrus Processor 2019.1.2. The ν-values are reported in units of reciprocal centimeters (cm⁻¹).

### Melting Points

Melting points (MP) were recorded using an Electrothermal™ IA9300 digital melting point apparatus. Samples were analyzed in 1.5 mm outer diameter capillaries with a sample height of 1 mm. The Tₘ-values values are uncorrected and reported in units of degrees Celsius (° C.).

### Example 15

### Synthesis of Ethyl 2-oxopent-4-enoate (EKP)

Protocol based on Zhang et al. Sci. Rep. 2017, 7 (1also).

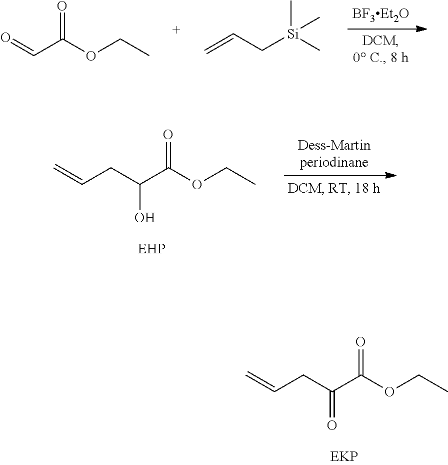

Ethyl 2-oxopent-4-enoate (ethyl ketopentanoate, EKP) was prepared by adding dropwise boron trifluoride diethyletherate (6.34 mL, 50.00 mmol, 1.00 equiv.) to a stirring solution of ethyl glyoxylate (˜50% in toluene, 9.91 mL, 50.00 mmol, 1.00 equiv.) and allyltrimethylsilane (15.89 mL, 100.00 mmol, 2.00 equiv.) in dry DCM (120 mL) at 0° C. The solution was allowed to warm up to ambient temperature and was stirred for an additional 8 h. After this time, the reaction was quenched with a saturated aqueous NH₄Cl and extracted with DCM (3×50 mL). The combined organic extracts were washed with brine (100 mL), dried over Na₂SO₄ and filtered. The filtrate was concentrated under reduced pressure to yield the crude ethyl 2-hydroxypent-4-enoate (ethyl hydroxypentanoate, EHP) intermediate as a yellow oil.

To a solution of this crude ethyl 2-hydroxypent-4-enoate in DCM (250 mL) was added Dess-Martin periodinane (24.56 g, 55.00 mmol, 1.10 equiv.) while stirring at room temperature. After 18 h, the mixture was quenched with a 1/1 mixture of 10% aqueous Na₂S₂O₃/saturated aqueous NaHCO₃ and extracted with DCM (3×50 mL). The combined organic layers were washed with water and brine, dried over Na₂SO₄ and filtered. The filtrate was concentrated under reduced pressure. To the residue was added Et₂O (100 mL) to precipitate out the Dess-Martin iodinane by-products. The suspension was filtered on a glass filter and the EKP-containing filtrate was concentrated under reduced pressure. The residue was purified via column chromatography on silica gel (95/5 pentane/Et₂O), furnishing the desired product as a light yellow oil in 26-38% yield.

¹H NMR (400 MHz, CDCl₃): δ 6.77 (ddd, J=17.2, 11.2, 10.3 Hz, 1H), 6.22 (ddd, J=11.2, 0.9, 0.8 Hz, 1H), 5.92 (br, s, 1H), 5.42 (ddd, J=17.2, 1.8, 0.9 Hz, 1H), 5.26 (ddd, J=10.3, 1.8, 0.8 Hz, 1H), 4.31 (q, J=7.1 Hz, 2H), 1.35 (t, J=7.1 Hz, 3H). ¹³C NMR (101 MHz, CDCl₃): δ 165.65, 139.38, 129.78, 120.16, 112.15, 62.35, 14.02. IR (neat): ν 3424.92 (O—H stretch), 3090.25 (C—H stretch), 2982.60 (C—H stretch), 2941.26 (C—H stretch), 2910.25 (C—H stretch), 1688.69 (C═O stretch). LR-MS (ESI): m/z calculated for [M+Na]⁺ 165.0528, found 165.1.

All data are in accordance with Zhang cited above. Note that EKP exists solely as its tautomeric enol form when dissolved in CDCl₃.

### General Notes on the Synthesis of EKP

EKP degrades at temperatures higher than 40° C. and in the presence of (Lewis) acids and nucleophiles. Therefore, rotary evaporation was always performed at 35° C. Furthermore, a significant portion of the yield is lost during column chromatography on silica gel. Hence, the elution time should be kept as short as possible. Other methods of purification (e.g. vacuum distillation and kugelrohr) proved even less successful.

Note that EKP is a highly toxic substance with a high vapor pressure. Care should be taken during the entire synthetic procedure or when handling the product. EKP should be stored at a temperature of −25° C. or lower, which freezes the compound. In this fashion, the thermal degradation rate is decreased and the vapor pressure is reduced.

### Example 16

### Synthesis of Propynones

### General Procedure A for the Synthesis of Propynones

Protocol based on Neumann et al. Org. Lett. 2014, 16 (8), 2216-2219 and Veryser et al. React. Chem. Eng. 2016, 1 (2), 142-146.

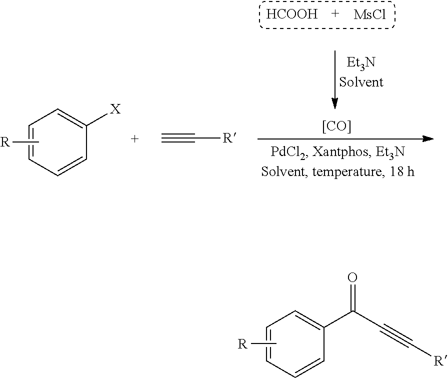

To the right chamber of a flame-dried two-chamber reactor (COWare)⁴⁻⁵ was added the aryl halide (0.50 mmol, 1.00 equiv.), palladium(II) chloride (4.4 mg, 0.025 mmol, 5.0 mol %) and Xantphos (14.5 mg, 0.025 mmol, 5.0 mol %) [Hermange et al. J. Am. Chem. Soc. 2011, 133 (15), 6061-6071; Friis et al. J. Am. Chem. Soc. 2011, 133 (45), 18114-18117]. The reactor was closed with two screw caps and septa and was evacuated and backfilled with argon three times. Dry, degassed toluene (3 mL) was added to the left chamber, followed by formic acid (29 μL, 0.75 mmol, 1.50 equiv.) and mesylchloride (58 μL, 0.75 mmol, 1.50 equiv.). To the right chamber, dry, degassed dioxane (3 mL) was added, followed by the terminal alkyne (0.75 mmol, 1.50 equiv.) and dry triethylamine (0.21 mL, 1.50 mmol, 3.00 equiv.) [If solid, the alkyne was added to the two-chamber reactor before it was closed with the screw cap]. The reaction was initiated by the addition of triethylamine (0.21 mL, 1.50 mmol, 3.00 equiv.) to the left chamber of the reactor. Immediately after the addition of the triethylamine, the reactor was placed in an oil bath at 80 or 100° C. for 18 h. When the reaction was finished, the crude reaction mixture was filtered over a pad of Celite® 535. The filtrate was concentrated in vacuo and purified via column chromatography on silica gel.

### General Procedure B for the Synthesis of Propynones

Protocol based on WO2012065963 and Yamaji et al. Phys. Chem. Chem. Phys. 2017, 19 (26), 17028-17035.

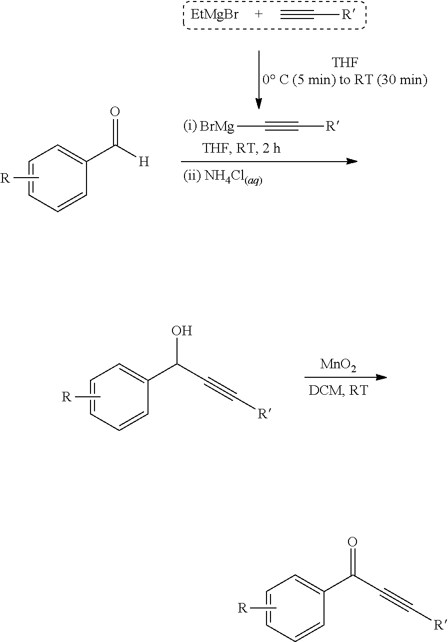

At 0° C., EtMgBr (3.0 M in Et₂O, 1.00 ml, 3.00 mmol, 3.00 equiv.) was added to a solution of the terminal alkyne (3.00 mmol, 3.0 equiv.) in dry THF (8 mL) and stirred for 5 min at 0° C. and then for 30 min at room temperature. This solution was slowly added to a solution of the aldehyde (1.00 mmol, 1.00 equiv.) in dry THF (10 mL) under a nitrogen atmosphere at room temperature. After 2 h, the mixture was quenched with saturated aqueous NH₄Cl, extracted with DCM and the organic layers were washed with brine and dried over Na₂SO₄. The resulting organic solution was filtered and the filtrate was concentrated under reduced pressure.

The crude residue was dissolved in DCM (10 mL) and stirred overnight at room temperature in the presence of manganese dioxide (609 mg, 7.00 mmol, 7.00 equiv.). The solution was filtered over Celite® 535 and the solvent was evaporated in vacuo. The residue was purified via column chromatography on silica gel.

### 1-(4-methoxyphenyl)-3-(p-tolyl)prop-2-yn-1-one (I.1)

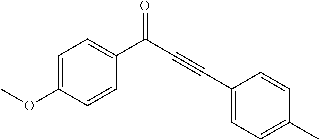

General procedure A for the synthesis of propynones was followed using 1-bromo-4-methoxybenzene (63 μL) and 1-ethynyl-4-methylbenzene (95 μL) at 80° C. In both chambers, 2 mL of solvent was used. The crude reaction mixture was purified via flash column chromatography on silica gel (heptane/EtOAc 95/5), furnishing 72% of the desired product as a yellow solid.

¹H NMR (300 MHz, CDCl₃): δ 8.23-8.14 (m, 2H), 7.57 (app. d, J=8.0 Hz, 2H), 7.21 (app. d, J=8.0 Hz, 2H), 7.01-6.94 (m, 2H), 3.89 (s, 3H), 2.40 (s, 3H). ¹³C NMR (75 MHz, CDCl₃): δ 176.77, 164.41, 141.32, 133.01, 131.96, 130.40, 129.47, 117.23, 113.86, 93.01, 86.78, 55.60, 21.77. IR (neat): ν 2995.00 (C—H stretch), 2968.13 (C—H stretch), 2837.91 (C—H stretch), 2188.89 (C≡C stretch), 1616.35 (C═O stretch). HR-MS (ESI): m/z calculated for [M+H]⁺ 251.1072, found 251.1071. MP: Tₘ 87.5-88.3.

All data are in accordance with CN107602361.

### 1-(4-nitrophenyl)-3-(p-tolyl)prop-2-yn-1-one (I.2)

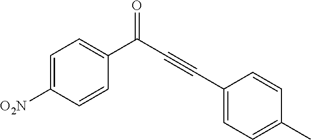 [SMILES: Cc1ccc(C#CC(=O)c2ccc([N+](=O)[O-])cc2)cc1]

General procedure A for the synthesis of propynones was followed using 1-bromo-4-nitrobenzene (101 mg) and 1-ethynyl-4-methylbenzene (95 μL) at 80° C. In both chambers, 2 mL of solvent was used. The crude reaction mixture was purified via flash column chromatography on silica gel (heptane/EtOAc 95/5), yielding 12% of the desired product as a yellow solid.

¹H NMR (300 MHz, CDCl₃): δ 8.37 (app. s, 4H), 7.61 (app. d, J=8.1 Hz, 2H), 7.27 (app. d, J=8.1 Hz, 2H), 2.43 (s, 3H). ¹³C NMR (75 MHz, CDCl₃): δ 175.92, 150.81, 142.44, 141.14, 133.39, 130.42, 129.69, 123.85, 116.27, 96.26, 86.56, 21.88. IR (neat): ν 3102.48 (C—H stretch), 2922.65 (C—H stretch), 2850.31 (C—H stretch), 2195.09 (C≡C stretch), 1641.15 (C═O stretch), 1510.93 (NO₂ stretch). HR-MS (ESI): m/z calculated for [M+H]⁺ 266.0817, found 266.0812. MP: Tₘ 159.6-160.7.

All data are in accordance with Sarkae et al. Appl. Organomet. Chem. 2020, 34 (7), e5646.

### 1-(2,3-dihydrobenzo[b][1,4]dioxin-6-yl)-3-phenylprop-2-yn-1-one (I.3)

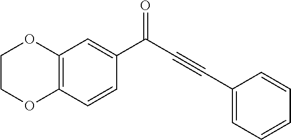 [SMILES: O=C(C#Cc1ccccc1)c1ccc2c(c1)OCCO2]

General procedure A for the synthesis of propynones was followed using 6-bromo-2,3-dihydrobenzo[b][1,4]dioxine (67 μL) and ethynylbenzene (82 μL) at 80° C. In both chambers, 2 mL of solvent was used. The crude reaction mixture was purified via flash column chromatography on silica gel (heptane/EtOAc 95/5), furnishing the desired compound as a beige solid in 43% yield.

¹H NMR (300 MHz, CDCl₃): δ 7.80-7.73 (m, 2H), 7.69-7.64 (m, 2H), 7.52-7.36 (m, 3H), 6.99-6.93 (m, 1H), 4.37-4.26 (m, 4H). ¹³C NMR (75 MHz, CDCl₃): δ 176.54, 149.15, 143.40, 133.01, 131.01, 130.66, 128.67, 123.95, 120.27, 118.87, 117.40, 92.38, 86.87, 64.78, 64.08. IR (neat): ν 3063.21 (C—H stretch), 2935.06 (C—H stretch), 2877.18 (C—H stretch), 2203.36 (C≡C stretch), 1630.82 (C═O stretch). HR-MS (ESI): m/z calculated for [M+H]⁺ 265.0865, found 265.0859. MP: Tₘ 98.1-100.8 (literature 98.5-99.7).

All data are in accordance with Levashov et al. Russ. J. Gen. Chem. 2017, 87(7), 1627-1630 and Wu et al. Chemistry—A European Journal 2010, 16 (40), 12104-12107.

### 1-(4-aminophenyl)-3-(4-(tert-butyl)phenyl)prop-2-yn-1-one (I.4)

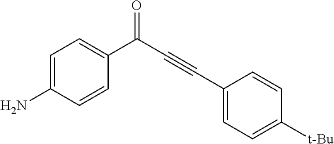 [SMILES: CC(C)(C)c1ccc(C#CC(=O)c2ccc(N)cc2)cc1]

General procedure A for the synthesis of propynones was followed using 4-bromoaniline (86 mg) and 1-(tert-butyl)-4-ethynylbenzene (135 μL) at 80° C. In both chambers, 2 mL of solvent was used. The crude reaction mixture was purified via flash column chromatography on silica gel (heptane/EtOAc 8/2), furnishing 64% of the desired product as a dark brown solid.

¹H NMR (400 MHz, CDCl₃): δ 8.05 (app. d, J=8.5 Hz, 2H), 7.58 (app. d, J=8.3 Hz, 2H), 7.41 (app. d, J=8.3 Hz, 2H), 6.67 (app. d, J=8.5 Hz, 2H), 4.37 (br, s, 2H), 1.32 (s, 9H). ¹³C NMR (101 MHz, CDCl₃): δ 176.51, 154.12, 152.49, 132.82, 132.40, 132.05, 125.74, 117.60, 113.79, 92.27, 87.00, 35.11, 31.15. IR (neat): ν 3350.51 (N—H stretch), 3222.36 (N—H stretch), 2957.79 (C—H stretch), 2864.78 (C—H stretch), 2190.96 (C≡C stretch), 1614.28 (C═O stretch). HR-MS (ESI): m/z calculated for [M+H]⁺ 278.1545, found 278.1539. MP: Tₘ 137.9-139.7.

### 1-(2-aminophenyl)-3-(4-(tert-butyl)phenyl)prop-2-yn-1-one (I.5)

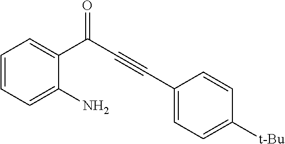 [SMILES: CC(C)(C)c1ccc(C#CC(=O)c2ccccc2N)cc1]

General procedure A for the synthesis of propynones was followed using 2-bromoaniline (86 mg) and 1-(tert-butyl)-4-ethynylbenzene (135 μL.) at 80° C. In both chambers, 2 mL of solvent was used. The crude reaction mixture was purified via flash column chromatography on silica gel (heptane/EtOAc 98/2), yielding 54% of the desired product as a yellow oil.

¹H NMR (400 MHz, CDCl₃): δ 8.20 (app. d, J=8.1 Hz, 1H), 7.61 (app. d, J=8.2 Hz, 2H), 7.43 (app. d, J=8.2 Hz, 2H), 7.31 (app. t, J=8.1 Hz, 1H), 6.73 (app. t, J=8.1 Hz, 1H), 6.67 (app. d, J=8.1 Hz, 1H), 6.39 (br, s, 2H), 1.34 (s, 9H). ¹³C NMR (101 MHz, CDCl₃): δ 179.75, 154.17, 151.16, 135.31, 134.59, 132.79, 125.77, 119.08, 117.58, 116.87, 116.19, 92.99, 87.02, 35.15, 31.18. IR (neat): ν 3445.59 (N—H stretch), 3338.11 (N—H stretch), 3036.34 (C—H stretch), 2961.93 (C—H stretch), 2904.05 (C—H stretch), 2866.85 (C—H stretch), 2199.23 (C≡C stretch), 1620.48 (C═O stretch). HR-MS (ESI): m/z calculated for [M+H]⁺ 278.1545, found 278.1540.

### 1-(2-aminophenyl)non-2-yn-1-one (I.6)

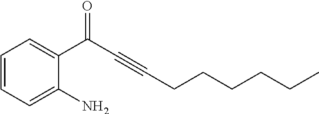 [SMILES: CCCCCCC#CC(=O)c1ccccc1N]

General procedure A for the synthesis of propynones was followed using 2-bromoaniline (86 mg) and oct-1-yne (149 μL, 1.00 mmol, 2.00 equiv.) at 100° C. In both chambers, 2 mL of solvent was used. The crude reaction mixture was purified via flash column chromatography on silica gel (heptane/EtOAc 95/5), furnishing the desired product as a brown oil in 48% yield.

¹H NMR (400 MHz, CDCl₃): δ 8.07 (app. dd, J=8.3, 1.0 Hz, 1H), 7.27 (app. t, J=7.2 Hz, 1H), 6.67 (app. t, J=7.2 Hz, 1H), 6.62 (app. d, J=8.3 Hz, 1H), 6.32 (br, s, 2H), 2.47 (t, J=7.1 Hz, 2H), 1.65 (app. quint, J=7.1 Hz, 2H), 1.46 (app. quint, J=7.1 Hz, 2H), 1.37-1.27 (m, 4H), 0.90 (t, J=6.8 Hz, 3H). ¹³C NMR (101 MHz, CDCl₃): δ 179.94, 150.96, 135.01, 134.65, 118.86, 116.67, 115.94, 95.85, 79.87, 31.25, 28.65, 27.86, 22.51, 19.20, 14.03. IR (neat): ν 3437.32 (N—H stretch), 3243.03 (N—H stretch), 2926.79 (C—H stretch), 2856.51 (C—H stretch), 2219.89 (C≡C stretch), 1618.41 (C═O stretch). HR-MS (ESI): m/z calculated for [M+H]⁺ 230.1545, found 230.1544.

### ethyl 4-(non-2-ynoyl)benzoate (I.7)

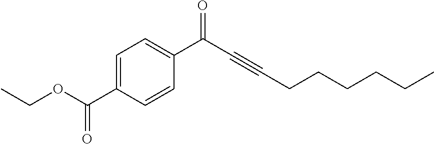 [SMILES: CCCCCCC#CC(=O)c1ccc(C(=O)OCC)cc1]

General procedure A for the synthesis of propynones was followed using ethyl 4-bromobenzoate (82 μL) and oct-1-yne (149 μL, 1.00 mmol, 2.00 equiv.) at 100° C. In both chambers, 2 mL of solvent was used. The crude reaction mixture was purified via flash column chromatography on silica gel (heptane/EtOAc 99/1), yielding 21% of the desired product as a brown oil.

¹H NMR (400 MHz, CDCl₃): δ 8.16 (app. d, J=8.5 Hz, 2H), 8.13 (app. d, J=8.5 Hz, 2H), 4.40 (q, J=7.1 Hz, 2H), 2.51 (t, J=7.1 Hz, 2H), 1.69 (app. quint, J=7.2 Hz, 2H), 1.53-1.46 (m, 2H), 1.41 (t, J=7.1 Hz, 3H), 1.38-1.29 (m, 4H), 0.90 (t, J=6.7 Hz, 3H). ¹³C NMR (101 MHz, CDCl₃): δ 177.47, 165.73, 139.94, 134.87, 129.65, 129.36, 98.10, 79.69, 61.52, 31.23, 28.66, 27.74, 22.50, 19.29, 14.28, 14.02. IR (neat): ν 2955.73 (C—H stretch), 2928.86 (C—H stretch), 2858.58 (C—H stretch), 2199.23 (C≡C stretch), 1719.69 (C═O stretch ester), 1647.35 (C═O stretch ketone). HR-MS (ESI): m/z calculated for [M+H]⁺ 287.1647, found 287.1638.

### 3-(4-(tert-butyl)phenyl)-1-(thiophen-3-yl)prop-2-yn-1-one (I.8)

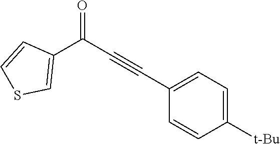 [SMILES: CC(C)(C)c1ccc(C#CC(=O)c2ccsc2)cc1]

General procedure A for the synthesis of propynones was followed using 3-bromothiophene (47 μL) and 1-(tert-butyl)-4-ethynylbenzene (180 μL, 1.00 mmol, 2.00 equiv.) at 100° C. In both chambers, 2 mL of solvent was used. The crude reaction mixture was purified via flash column chromatography on silica gel (heptane/EtOAc 98/2 to 9/1), furnishing 39% of the desired product as a brown oil.

¹H NMR (400 MHz, CDCl₃): δ 8.36 (app. dd, J=2.9, 0.6 Hz, 1H), 7.68 (app. dd, J=5.1, 0.6 Hz, 1H), 7.61 (app. d, J=8.2 Hz, 2H), 7.45 (d, J=8.2 Hz, 2H), 7.34 (app. dd, J=5.1, 3.0 Hz, 1H), 1.35 (s, 9H). ¹³C NMR (101 MHz, CDCl₃): δ 171.53, 154.57, 143.14, 135.26, 132.95, 126.87, 126.77, 125.79, 116.99, 92.04, 87.20, 35.11, 31.08. IR (neat): ν 3106.61 (C—H stretch), 2961.93 (C—H stretch), 2866.85 (C—H stretch), 2186.82 (C≡C stretch), 1626.68 (C═O stretch). HR-MS (ESI): m/z calculated for [M+H]⁺ 269.1000, found 269.0997.

### 1-(thiophen-3-yl)non-2-yn-1-one (I.9)

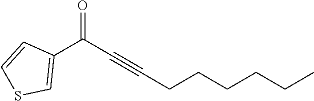

General procedure A for the synthesis of propynones was followed using 3-bromothiophene (47 μL) and oct-1-yne (149 μL, 1.00 mmol, 2.00 equiv.) at 100° C. In both chambers, 2 mL of solvent was used. The crude reaction mixture was purified via flash column chromatography on silica gel (heptane/EtOAc 98/2 to 95/5), furnishing the desired product as a brown oil in 53% yield.

¹H NMR (300 MHz, CDCl₃): δ 8.22 (app. dd, J=3.0, 1.2 Hz, 1H), 7.59 (app. dd, J=5.1, 1.2 Hz, 1H), 7.30 (app. dd, J=5.1, 3.0 Hz, 1H), 2.45 (t, J=7.1 Hz, 2H), 1.66 (app. quint, J=7.1 Hz, 2H), 1.52-1.40 (m, 2H), 1.39-1.26 (m, 4H), 0.98 (t, J=6.8 Hz, 3H). ¹³C NMR (75 MHz, CDCl₃): δ 171.71, 143.13, 135.19, 126.77, 126.59, 94.98, 80.18, 31.22, 28.63, 27.78, 22.48, 19.10, 14.02. IR (neat): ν 3106.61 (C—H stretch), 2953.66 (C—H stretch), 2926.79 (C—H stretch), 2856.51 (C—H stretch), 2199.23 (C≡C stretch), 1632.88 (C═O stretch). HR-MS (ESI): m/z calculated for [M+H]⁺ 221.1000, found 221.0995.

### 3-cyclopropyl-1-(thiophen-3-yl)prop-2-yn-1-one (I.10)

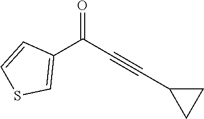 [SMILES: O=C(C#CC1CC1)c1ccsc1]

General procedure A for the synthesis of propynones was followed using 3-bromothiophene (47 μL) and ethynylcyclopropane (85 μL, 1.00 mmol, 2.00 equiv.) at 100° C. In both chambers, 2 mL of solvent was used. The crude reaction mixture was purified via flash column chromatography on silica gel (heptane/EtOAc 98/2 to 8/2), furnishing 60% of the desired product as a brown solid.

¹H NMR (300 MHz, CDCl₃): δ 8.19 (app. dd, J=3.0, 1.2 Hz, 1H), 7.56 (app. dd, J=5.1, 1.2 Hz, 1H), 7.29 (app. dd, J=5.1, 3.0 Hz, 1H), 1.57-1.45 (m, 1H), 1.07-0.95 (m, 4H). ¹³C NMR (75 MHz, CDCl₃): δ 171.55, 143.14, 135.04, 126.84, 126.63, 99.20, 76.00, 9.91, −0.01. IR (neat): ν 3085.94 (C—H stretch), 3005.33 (C—H stretch), 2920.59 (C—H stretch), 2850.31 (C—H stretch), 2207.49 (C≡C stretch), 1661.82 (C═O stretch). HR-MS (ESI): m/z calculated for [M+H]⁺ 177.0374, found 177.0368. MP: Tₘ 78.8-80.6.

### 3-(4-(tert-butyl)phenyl)-1-(4-methylpyridin-3-yl)prop-2-yn-1-one (I.11)

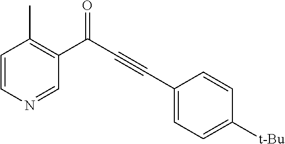 [SMILES: Cc1ccncc1C(=O)C#Cc1ccc(C(C)(C)C)cc1]

General procedure A for the synthesis of propynones was followed using 3-bromo-4-methylpyridine (56 μL) and 1-(tert-butyl)-4-ethynylbenzene (180 μL, 1.00 mmol, 2.00 equiv.) at 100° C. In both chambers, 2 mL of solvent was used. The crude reaction mixture was purified via flash column chromatography on silica gel (DCM to DCM/MeOH 95/5), furnishing the desired product as a dark brown oil in 15% yield.

¹H NMR (300 MHz, CDCl₃) δ 9.46 (app. s, 1H), 8.60 (app. d, J=5.1 Hz, 1H), 7.62 (app. d, J=8.6 Hz, 2H), 7.44 (app. d, J=8.6 Hz, 2H), 7.21 (app. d, J=5.1 Hz, 1H), 2.68 (s, 3H), 1.33 (s, 9H). ¹³C NMR (75 MHz, CDCl₃): δ 178.19, 154.90, 153.85, 152.58, 149.44, 133.10, 131.85, 126.82, 125.83, 116.65, 94.00, 87.60, 35.14, 31.04, 21.36. IR (neat): ν 3046.67 (C—H stretch), 2961.93 (C—H stretch), 2906.12 (C—H stretch), 2868.91 (C—H stretch), 2195.09 (C≡C stretch), 1641.15 (C═O stretch). HR-MS (ESI): m/z calculated for [M+H]⁺ 278.1545, found 278.1541.

### 3-(4-(tert-butyl)phenyl)-1-(isoquinolin-4-yl)prop-2-yn-1-one (I.12)

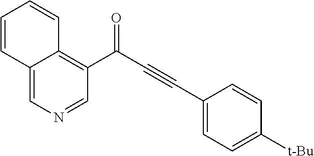 [SMILES: CC(C)(C)c1ccc(C#CC(=O)c2cncc3ccccc23)cc1]

General procedure A for the synthesis of propynones was followed using 4-bromoisoquinoline (104 mg) and 1-(tert-butyl)-4-ethynylbenzene (180 μL, 1.00 mmol, 2.00 equiv.) at 100° C. In both chambers, 2 mL of solvent was used. The crude reaction mixture was purified via flash column chromatography on silica gel (DCM to DCM/MeOH 95/5), furnishing 41% of the desired product as a dark yellow solid.

¹H NMR (400 MHz, CDCl₃): δ 9.61 (app. s, 1H), 9.41 (app. s, 1H), 9.20 (app. d, J=8.7 Hz, 1H), 8.06 (app. d, J=8.2 Hz, 1H), 7.89 (app. t, J=7.8 Hz, 1H), 7.71 (app. t, J=7.5 Hz, 1H), 7.66 (app. d, J=8.3 Hz, 2H), 7.46 (app. d, J=8.3 Hz, 2H), 1.34 (s, 9H). ¹³C NMR (101 MHz, CDCl₃): δ 178.87, 158.07, 154.94, 150.65, 133.40, 133.24, 131.62, 128.60, 128.49, 128.26, 126.66, 125.96, 125.45, 116.92, 93.58, 87.77, 35.26, 31.17. IR (neat): ν 3048.74 (C—H stretch), 2957.79 (C—H stretch), 2904.05 (C—H stretch), 2864.78 (C—H stretch), 2193.02 (C≡C stretch), 1628.75 (C═O stretch). HR-MS (ESI): m/z calculated for [M+H]⁺ 314.1545, found 314.1541. MP: Tₘ 102.5-104.4.

### 1-(2-amino-5-methylphenyl)-3-(4-(tert-butyl)phenyl)prop-2-yn-1-one (I.13)

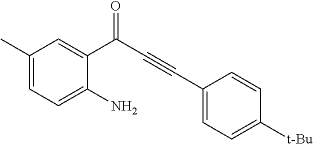 [SMILES: Cc1ccc(N)c(C(=O)C#Cc2ccc(C(C)(C)C)cc2)c1]

General procedure A for the synthesis of propynones was followed using 2-bromo-4-methylaniline (62 μL) and 1-(tert-butyl)-4-ethynylbenzene (180 μL, 1.00 mmol, 2.00 equiv.) at 100° C. In both chambers, 2 mL of solvent was used. The crude reaction mixture was purified via flash column chromatography on silica gel (heptane/EtOAc 95/5), furnishing the title compound as a dark brown oil in 29% yield.

¹H NMR (400 MHz, CDCl₃): δ 7.96 (s, 1H), 7.61 (app. d, J=8.3 Hz, 2H), 7.44 (app. d, J=8.3 Hz, 2H), 7.15 (app. d, J=8.4 Hz, 1H), 6.60 (app. d, J=8.4 Hz, 1H), 6.25 (br, s, 2H), 2.30 (s, 3H), 1.35 (s, 9H). ¹³C NMR (101 MHz, CDCl₃): δ 179.51, 153.97, 149.08, 136.58, 133.79, 132.60, 125.65, 125.06, 118.85, 117.60, 116.88, 92.72, 87.01, 35.03, 31.08, 20.42. IR (neat): ν 3445.59 (N—H stretch), 3333.98 (N—H stretch), 3036.34 (C—H stretch), 2959.86 (C—H stretch), 2904.05 (C—H stretch), 2866.85 (C—H stretch), 2190.96 (C≡C stretch), 1628.75 (C═O stretch). LR-MS (ESI): m/z calculated for [M+H]⁺ 292.1701, found 292.2.

All data are in accordance with Veryser et al. Adv. Synth. Catal. 2017, 359 (8), 1271-1276.

### 3-(4-(tert-butyl)phenyl)-1-(2-(methylamino)phenyl)prop-2-yn-1-one (I.14)

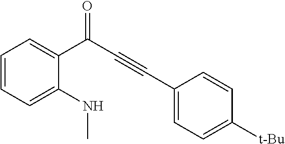 [SMILES: CNc1ccccc1C(=O)C#Cc1ccc(C(C)(C)C)cc1]

General procedure A for the synthesis of propynones was followed using 2-bromo-N-methylaniline (59 μL) and 1-(tert-butyl)-4-ethynylbenzene (180 μL, 1.00 mmol, 2.00 equiv.) at 100° C. In both chambers, 2 mL of solvent was used. The crude reaction mixture was purified via flash column chromatography on silica gel (heptane/EtOAc 98/2), furnishing the desired product as a dark yellow solid in 41% yield.

¹H NMR (400 MHz, CDCl₃): δ 8.80 (br, d, J=5.1 Hz, 1H), 8.24 (app. dd, J=8.0, 1.2 Hz, 1H), 7.61 (app. d, J=8.4 Hz, 2H), 7.45-7.38 (m, 3H), 6.72-6.63 (m, 2H), 2.97 (d, J=5.1 Hz, 3H), 1.35 (s, 9H). ¹³C NMR (151 MHz, CDCl₃): δ 179.49, 153.93, 152.75, 135.90, 135.29, 132.63, 125.65, 118.59, 117.65, 114.45, 111.02, 92.80, 86.98, 35.07, 31.11, 29.35. IR (neat): ν 3317.44 (N—H stretch), 3081.81 (C—H stretch), 2955.73 (C—H stretch), 2924.72 (C—H stretch), 2868.91 (C—H stretch), 2819.31 (C—H stretch), 2199.23 (C≡C stretch), 1616.35 (C═O stretch). HR-MS (ESI): m/z calculated for [M+H]⁺ 292.1701, found 292.1696. MP: Tₘ 141.6-142.8.

### 1-(2-aminopyridin-3-yl)-3-phenylprop-2-yn-1-one (1.1)

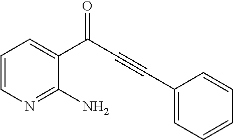 [SMILES: Nc1ncccc1C(=O)C#Cc1ccccc1]

General procedure A for the synthesis of propynones was followed using 3-bromopyridin-2-amine (88 mg) and ethynylbenzene (87 μL) at 100° C. In both chambers, 2 mL of solvent was used. The crude reaction mixture was purified via flash column chromatography on silica gel (heptane/EtOAc 9/1 to 6/4), furnishing the desired product as a dark yellow solid in 41% yield.

¹H NMR (300 MHz, CDCl₃): δ 8.46 (app. dd, J=7.8, 1.9 Hz, 1H), 8.29 (app. dd, J=4.7, 1.9 Hz, 1H), 7.69-7.64 (m, 2H), 7.53-739 (m, 3H), 6.74 (app. dd, J=7.8, 4.7 Hz, 1H). ¹³C NMR (151 MHz, CDCl₃): δ 178.43, 159.15, 155.21, 143.26, 133.04, 130.90, 128.86, 120.25, 114.36, 112.99, 93.66, 86.50. IR (neat): ν 3400.12 (N—H stretch), 3253.37 (N—H stretch), 3092.14 (C—H stretch), 2916.45 (C—H stretch), 2850.31 (C—H stretch), 2190.96 (C≡C stretch), 1614.28 (C═O stretch). HR-MS (ESI): m/z calculated for [M+H]⁺ 223.0871, found 223.0864. MP: Tₘ 146.2-147.8.

### 1-(2-aminopyridin-3-yl)-3-(4-fluorophenyl)prop-2-yn-1-one (1.2)

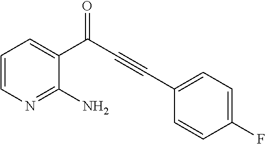 [SMILES: Nc1ncccc1C(=O)C#Cc1ccc(F)cc1]

General procedure A for the synthesis of propynones was followed using 3-bromopyridin-2-amine (88 mg) and 1-ethynyl-4-fluorobenzene (87 μL) at 100° C. In both chambers, 2 mL of solvent was used. The crude reaction mixture was purified via flash column chromatography on silica gel (heptane/EtOAc 8/2 to 7/3), yielding 50% of the desired product as a light brown solid.

¹H NMR (400 MHz, CDCl₃): δ 8.42 (app. dd, J=7.9, 1.9 Hz, 1H), 8.29 (app. dd, J=4.7, 1.9 Hz, 1H), 7.70-7.63 (m, 2H), 7.16-7.09 (m, 2H), 6.73 (app. dd, J=7.9, 4.7 Hz, 1H). ¹³C NMR (101 MHz, CDCl₃): δ 178.26, 164.13 (d, J=253.8 Hz), 159.15, 155.25, 143.17, 135.29 (d, J=8.8 Hz), 116.41 (d, J=22.2 Hz), 116.35 (d, J=3.7 Hz), 114.24, 112.96, 92.55, 86.39. IR (neat): ν 3398.05 (N—H stretch), 3263.70 (N—H stretch), 3112.81 (C—H stretch), 2924.72 (C—H stretch), 2190.96 (C≡C stretch), 1620.48 (C═O stretch). HR-MS (ESI): m/z calculated for [M+H]⁺ 241.0777, found 241.0770. MP: Tₘ 172.5-173.5.

### 1-(2-aminopyridin-3-yl)-3-(triisopropylsilyl)prop-2-yn-1-one (1.3)

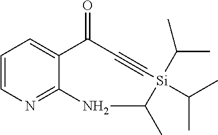 [SMILES: CC(C)[Si]1(C(C)C)C#CC(=O)c2cccnc2NC1C]

General procedure A for the synthesis of propynones was followed using 3-bromopyridin-2-amine (88 mg) and ethynyltriisopropylsilane (173 μL) at 80° C. The crude reaction mixture was purified via flash column chromatography on silica gel (heptane/EtOAc 8/2 to 7/3), yielding 23% of the desired product as a bright yellow solid.

¹H NMR (300 MHz, CDCl₃): δ 8.41 (app. dd, J=7.9, 1.9 Hz, 1H), 8.27 (app. dd, J=4.7, 1.9 Hz, 1H), 6.70 (app. dd, J=7.9, 4.7 Hz, 1H), 1.16 (sept, J=4.2 Hz, 3H), 1.15 (d, J=4.6 Hz, 18H). ¹³C NMR (101 MHz, CDCl₃): δ 177.94, 159.12, 154.99, 143.31, 114.28, 112.98, 102.65, 98.50, 18.72, 11.26. IR (neat): ν 3395.98 (N—H stretch), 3271.97 (N—H stretch), 3125.22 (C—H stretch), 2945.39 (C—H stretch), 2862.71 (C—H stretch), 2151.69 (C≡C stretch), 1612.21 (C═O stretch). HR-MS (ESI): m/z calculated for [M+H]⁺ 303.1893, found 303.1889. MP: Tₘ 94.6-95.7.

### 1-(2-aminopyridin-3-yl)-3-cyclohexylprop-2-yn-1-one (1.4)

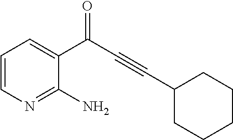 [SMILES: Nc1ncccc1C(=O)C#CC1CCCCC1]

General procedure A for the synthesis of propynones was followed using 3-bromopyridin-2-amine (88 mg) and ethynylcyclohexane (100 μL) at 80° C. The crude reaction mixture was purified via flash column chromatography on silica gel (DCM/EtOAc 90/10), followed by column chromatography on silica gel (DCM to DCM/EtOAc/MeOH 8/1/1), yielding 56% of the title compound as a bright yellow solid.

¹H NMR (400 MHz, CDCl₃): δ 8.34 (app. dd, J=7.8, 1.8 Hz, 1H), 8.25 (app. dd, J=4.7, 1.8 Hz, 1H), 6.69 (app. dd, J=7.8, 4.7 Hz, 1H), 2.74-2.62 (m, 1H), 1.98-1.86 (m, 2H), 1.82-1.70 (m, 2H), 1.69-1.49 (m, 4H), 1.46-1.32 (m, 3H). ¹³C NMR (151 MHz, CDCl₃): δ 178.89, 159.09, 154.76, 143.43, 114.43, 112.84, 100.82, 79.17, 31.86, 29.53, 25.78, 24.89. IR (neat): ν 3398.05 (N—H stretch), 3265.77 (N—H stretch), 3125.22 (C—H stretch), 2924.72 (C—H stretch), 2852.38 (C—H stretch), 2201.29 (C≡C stretch), 1608.08 (C═O stretch). HR-MS (ESI): m/z calculated for [M+H]⁺ 229.1341, found 229.1335. MP: Tₘ 137.5-138.6.

### 1-(2-aminopyridin-3-yl)oct-2-yn-1-one (1.5)

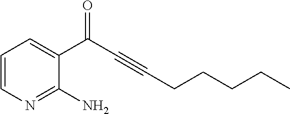 [SMILES: CCCCCC#CC(=O)c1cccnc1N]

General procedure A for the synthesis of propynones was followed using 3-bromopyridin-2-amine13 (88 mg) and hept-1-yne (100 μL) at 80° C. The crude reaction mixture was purified via column chromatography on silica gel (DCM/EtOAc 9/1 to 8/2), yielding 29% of the desired product as a bright yellow solid.

¹H NMR (400 MHz, CDCl₃): δ 8.34 (app. dd, J=7.8, 1.8 Hz, 1H), 8.25 (app. dd, J=4.7, 1.8 Hz, 1H), 6.69 (app. dd, J=7.8, 4.7 Hz, 1H), 2.49 (t, J=7.2 Hz, 2H), 1.73-1.62 (m, 2H), 1.50-1.31 (m, 4H), 0.93 (t, J=7.2 Hz, 3H). ¹³C NMR (151 MHz, CDCl₃): δ 178.80, 159.11, 154.85, 143.43, 114.35, 112.85, 97.35, 79.30, 31.28, 27.66, 22.27, 19.32, 14.06. IR (neat): ν 3400.12 (N—H stretch), 3259.57 (N—H stretch), 3116.95 (C—H stretch), 2959.86 (C—H stretch), 2930.92 (C—H stretch), 2852.38 (C—H stretch), 2211.63 (C≡C stretch), 1610.15 (C═O stretch). HR-MS (ESI): m/z calculated for [M+H]⁺ 217.1341, found 217.1337. MP: Tₘ 110.7-111.7.

### 1-(2-aminopyridin-3-yl)-4-methylpent-2-yn-1-one (1.6)

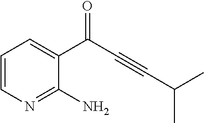

General procedure A for the synthesis of propynones was followed using 3-bromopyridin-2-amine (88 mg) and 3-methylbut-1-yne (81 μL) at 80° C. The crude reaction mixture was purified via column chromatography on silica gel (DCM/MeOH 99.5/0.5), yielding 26% of the desired product as a dark yellow solid.

¹H NMR (400 MHz, CDCl₃): δ 8.33 (app. dd, J=7.8, 1.8 Hz, 1H), 8.25 (app. dd, J=4.7, 1.8 Hz, 1H), 6.69 (app. dd, J=7.8, 4.7 Hz, 1H), 2.85 (sept, J=7.0 Hz, 1H), 1.32 (d, J=6.9 Hz, 6H). ¹³C NMR (151 MHz, CDCl₃): δ 178.85, 159.10, 154.84, 143.42, 114.33, 112.84, 101.86, 78.38, 22.18, 21.12. IR (neat): ν 3416.65 (N—H stretch), 3271.97 (N—H stretch), 3131.42 (C—H stretch), 2970.19 (C—H stretch), 2926.79 (C—H stretch), 2868.91 (C—H stretch), 2203.36 (C≡C stretch), 1614.28 (C═O stretch). HR-MS (ESI): m/z calculated for [M+H]⁺ 189.1028, found 189.1023. MP: Tₘ 91.7-92.8.

### 1-(2-aminopyridin-3-yl)-3-(4-chlorophenyl)prop-2-yn-1-one (1.7)

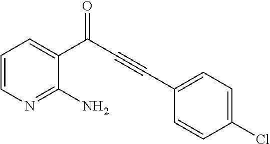 [SMILES: Nc1ncccc1C(=O)C#Cc1ccc(Cl)cc1]

General procedure A for the synthesis of propynones was followed using 3-bromopyridin-2-amine (88 mg) and 1-chloro-4-ethynylbenzene (102 mg) at 80° C. The crude reaction mixture was purified via column chromatography on silica gel (DCM), yielding 35% of the desired product as a bright yellow solid.

¹H NMR (600 MHz, CDCl₃): δ 8.43 (app. dd, J=7.8, 1.8 Hz, 1H), 8.32 (app. dd, J=4.7, 1.8 Hz, 1H), 7.62 (app. dd, J=8.6, 2.0 Hz, 2H), 7.43 (app. dd, J=8.6, 2.0 Hz, 2H), 6.76 (app. dd, J=7.8, 4.7 Hz, 1H). ¹³C NMR (151 MHz, CDCl₃): δ 178.13, 159.15, 155.35, 143.17, 137.29, 134.20, 129.33, 118.69, 114.19, 112.98, 92.19, 87.18. IR (neat): ν 3412.52 (N—H stretch), 3261.63 (N—H stretch), 3085.94 (C—H stretch), 2916.45 (C—H stretch), 2848.24 (C—H stretch), 2205.43 (C≡C stretch), 1610.15 (C═O stretch). HR-MS (ESI): m/z calculated for [M+H]⁺ 257.0482, found 257.0474. MP: Tₘ 183.3-184.2.

### methyl 4-(3-(2-aminopyridin-3-yl)-3-oxoprop-1-yn-1-yl)benzoate (1.8)

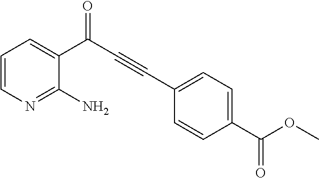 [SMILES: COC(=O)c1ccc(C#CC(=O)c2cccnc2N)cc1]

General procedure A for the synthesis of propynones was followed using 3-bromopyridin-2-amine (88 mg) and methyl 4-ethynylbenzoate (123 mg) at 80° C. The crude reaction mixture was purified via column chromatography on silica gel (Et₂O), furnishing the desired product as a dark yellow solid in 18% yield.

¹H NMR (400 MHz, CDCl₃): δ 8.43 (app. dd, J=7.9, 1.8 Hz, 1H), 8.31 (app. dd, J=4.7, 1.8 Hz, 1H), 8.09 (app. d, J=8.3 Hz, 2H), 7.72 (app. d, J=8.3 Hz, 2H), 6.74 (app. dd, J=7.9, 4.7 Hz, 1H), 3.95 (s, 3H). ¹³C NMR (151 MHz, CDCl₃): δ 178.00, 166.23, 159.17, 155.50, 143.21, 132.83, 131.88, 129.88, 124.71, 114.16, 113.04, 91.90, 88.19, 52.63. IR (neat): ν 3418.72 (N—H stretch), 3259.57 (N—H stretch), 3116.95 (C—H stretch), 2922.65 (C—H stretch), 2852.38 (C—H stretch), 2203.36 (C≡C stretch), 1719.69 (C═O stretch ester), 1612.21 (C═O stretch ketone). HR-MS (ESI): m/z calculated for [M+H]⁺ 281.0926, found 281.0926. MP: Tₘ 182.4-183.7 (decomposition).

### 1-(2-aminopyridin-3-yl)-3-(4-methoxyphenyl)prop-2-yn-1-one (1.9)

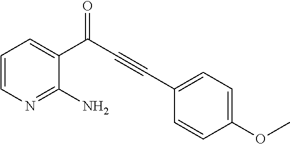 [SMILES: COc1ccc(C#CC(=O)c2cccnc2N)cc1]

General procedure A for the synthesis of propynones was followed using 3-bromopyridin-2-amine (88 mg) and 1-ethynyl-4-methoxybenzene (100 mg) at 80° C. Dry, degassed dioxane was used in both chambers. The crude reaction mixture was purified via column chromatography on silica gel (DCM/Et₂O 95/5 to 9/1), yielding 73% of the desired product as a yellow solid.

¹H NMR (400 MHz, CDCl₃): δ 8.44 (app. dd, J=7.8, 1.8 Hz, 1H), 8.27 (app. dd, J=4.7, 1.8 Hz, 1H), 7.65-7.58 (m, 2H), 7.10-6.78 (m, 2H), 6.72 (app. dd, J=7.8, 4.7 Hz, 1H), 3.85 (s, 3H). ¹³C NMR (101 MHz, CDCl₃): δ 178.50, 161.81, 159.14, 154.90, 143.17, 135.04, 114.57, 114.42, 112.86, 111.99, 94.73, 86.39, 55.58. IR (neat): ν 3402.19 (N—H stretch), 3261.63 (N—H stretch), 3131.42 (C—H stretch), 2930.92 (C—H stretch), 2835.84 (C—H stretch), 2188.89 (C≡C stretch), 1599.81 (C═O stretch). HR-MS (ESI): m/z calculated for [M+H]⁺ 253.0977, found 253.0972. MP: Tₘ 175.7-177.0.

### 1-(2-aminopyridin-3-yl)-3-(3-chlorophenyl)prop-2-yn-1-one (1.10)

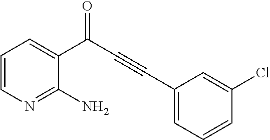 [SMILES: Nc1ncccc1C(=O)C#Cc1cccc(Cl)c1]

General procedure A for the synthesis of propynones was followed using 3-bromopyridin-2-amine (88 mg) and 1-chloro-3-ethynylbenzene (96 μL) at 80° C. Dry, degassed dioxane was used in both chambers. The crude reaction mixture was purified via column chromatography on silica gel (DCM/MeOH 99.9/0.1). Spectroscopic analysis of the product fraction indicated the presence of some residual aryl halide. Therefore, the product mixture was dissolved in Et₂O and subsequently washed with 0.5 M aqueous HCl, saturated aqueous NaHCO₃, 0.5 M aqueous HCl, saturated aqueous NaHCO₃ and brine. The organic phase was dried over MgSO₄, filtered and concentrated in vacuo. The title compound was obtained as a dark yellow solid in 23% yield.

¹H NMR (300 MHz, CDCl₃): δ 8.41 (app. dd, J=7.9, 1.9 Hz, 1H), 8.30 (app. dd, J=4.7, 1.9 Hz, 1H), 7.65 (app. t, J=1.7 Hz, 1H), 7.55 (app. dd, J=7.5, 2.6 Hz, 1H), 7.47 (app. ddd, J=8.1, 2.1, 1.2 Hz, 1H), 7.37 (app. t, J=7.8 Hz, 1H), 6.74 (app. dd, J=7.9, 4.7 Hz, 1H). ¹³C NMR (151 MHz, CDCl₃): δ 178.01, 159.15, 155.40, 143.22, 134.80, 132.64, 131.14, 131.09, 130.13, 121.96, 114.18, 113.03, 91.53, 86.99. IR (neat): ν 3418.72 (N—H stretch), 3267.83 (N—H stretch), 3112.81 (C—H stretch), 2920.59 (C—H stretch), 2852.38 (C—H stretch), 2199.23 (C≡C stretch), 1614.28 (C═O stretch). HR-MS (ESI): m/z calculated for [M+H]⁺ 257.0482, found 257.0475. MP: Tₘ 150.1-150.8.

### 1-(2-aminopyridin-3-yl)-3-(4-(trifluoromethyl)phenyl)prop-2-yn-1-one (1.11)

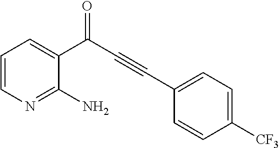 [SMILES: Nc1ncccc1C(=O)C#Cc1ccc(C(F)(F)F)cc1]

General procedure A for the synthesis of propynones was followed using 3-bromopyridin-2-amine (88 mg) and 1-ethynyl-4-(trifluoromethyl)benzene (125 μL) at 80° C. Dry, degassed dioxane was used in both chambers. The crude reaction mixture was purified via flash column chromatography on silica gel (DCM to DCM/EtOAc 95/5). Spectroscopic analysis of the product fraction indicated the presence of some residual aryl halide. Therefore, the product mixture was dissolved in Et₂O and subsequently washed with 0.5 M aqueous HCl, saturated aqueous NaHCO₃, 0.5 M aqueous HCl, saturated aqueous NaHCO₃ and brine. The organic phase was dried over MgSO₄, filtered and concentrated in vacuo. The title compound was obtained as a dark yellow oil in 21% yield.

¹H NMR (600 MHz, CDCl₃): δ 8.42 (app. dd, J=7.9, 1.9 Hz, 1H), 8.30 (app. dd, J=4.7, 1.9 Hz, 1H), 7.77 (app. d, J=8.1 Hz, 2H), 7.69 (app. d, J=8.1 Hz, 2H), 6.75 (app. dd, J=7.9, 4.7 Hz, 1H). ¹³C NMR (151 MHz, CDCl₃): δ 177.73, 159.01, 155.29, 143.11, 133.01, 132.25 (q, J=33.0 Hz), 125.66 (q, J=3.8 Hz), 123.90, 123.55 (q, J=272.5 Hz), 113.99, 112.87, 91.01, 87.58. IR (neat): ν 3404.25 (N—H stretch), 3278.17 (N—H stretch), 3131.42 (C—H stretch), 2955.73 (C—H stretch), 2920.59 (C—H stretch), 2852.38 (C—H stretch), 2207.49 (C≡C stretch), 1612.21 (C═O stretch). HR-MS (ESI): m/z calculated for [M+H]⁺ 291.0745, found 291.0739.

### 1-(2-aminopyridin-3-yl)-3-(p-tolyl)prop-2-yn-1-one (1.12)

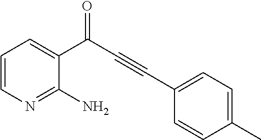 [SMILES: Cc1ccc(C#CC(=O)c2cccnc2N)cc1]

General procedure A for the synthesis of propynones was followed using 3-bromopyridin-2-amine (88 mg) and 1-ethynyl-4-methylbenzene (97 μL) at 80° C. Dry, degassed dioxane was used in both chambers. The crude reaction mixture was purified via flash column chromatography on silica gel (DCM/EtOAc 95/5 to 9/1), yielding 74% of the desired product as a bright yellow solid.

¹H NMR (400 MHz, CDCl₃): δ 8.45 (app. dd, J=7.8, 1.9 Hz, 1H), 8.28 (app. dd, J=4.7, 1.9 Hz, 1H), 7.56 (app. d, J=8.0 Hz, 2H), 7.23 (app. d, J=8.0 Hz, 2H), 6.73 (app. dd, J=7.8, 4.7 Hz, 1H), 2.41 (s, 3H). ¹³C NMR (101 MHz, CDCl₃): δ 178.52, 159.13, 155.05, 143.27, 141.64, 133.07, 129.65, 117.12, 114.43, 112.95, 94.34, 86.36, 21.92. IR (neat): ν 3418.72 (N—H stretch), 3255.43 (N—H stretch), 3119.01 (C—H stretch), 3034.27 (C—H stretch), 2916.45 (C—H stretch), 2850.31 (C—H stretch), 2197.16 (C≡C stretch), 1606.01 (C═O stretch). HR-MS (ESI): m/z calculated for [M+H]⁺ 237.1028, found 237.1022. MP: Tₘ 158.3-159.2.

### 1-(2-aminopyridin-3-yl)-3-(3,4-dichlorophenyl)prop-2-yn-1-one (1.13)

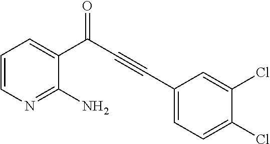 [SMILES: Nc1ncccc1C(=O)C#Cc1ccc(Cl)c(Cl)c1]

General procedure A for the synthesis of propynones was followed using 3-bromopyridin-2-amine (88 mg) and 1,2-dichloro-4-ethynylbenzene (132 mg) at 80° C. Dry, degassed dioxane was used in both chambers. The crude reaction mixture was purified via flash column chromatography on silica gel (DCM to DCM/EtOAc 9/1 to 6/4), followed by column chromatography on silica gel (DCM/EtOAc 8/2), furnishing the desired product as a bright yellow solid in 17% yield.

¹H NMR (400 MHz, CDCl₃): δ 8.38 (app. dd, J=7.9, 1.9 Hz, 1H), 8.31 (app. dd, J=4.7, 1.9 Hz, 1H), 7.75 (app. dd, J=1.7, 0.4 Hz, 1H), 7.51 (app. dd, J=8.3, 0.4 Hz, 1H), 7.48 (app. dd, J=8.3, 1.7 Hz, 1H), 6.74 (app. dd, J=7.9, 4.7 Hz, 1H). ¹³C NMR (101 MHz, CDCl₃): δ 177.80, 159.15, 155.55, 143.12, 135.70, 134.40, 133.38, 131.93, 131.03, 120.15, 114.07, 113.06, 90.43, 87.58. IR (neat): ν 3418.72 (N—H stretch), 3263.70 (N—H stretch), 3104.55 (C—H stretch), 3026.00 (C—H stretch), 2197.16 (C≡C stretch), 1614.28 (C═O stretch). HR-MS (ESI): m/z calculated for [M+H]⁺ 291.0092, found 291.0085. MP: Tₘ 182.1-182.9.

### 1-(2-aminophenyl)-3-(4-(tert-butyl)phenyl)prop-2-yn-1-one (1.14)

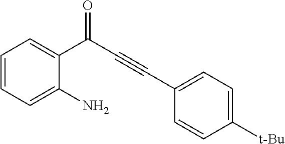 [SMILES: CC(C)(C)c1ccc(C#CC(=O)c2ccccc2N)cc1]

General procedure A for the synthesis of propynones was followed using 2-bromoaniline (58 μL) and 1-(tert-butyl)-4-ethynylbenzene (141 μL) at 80° C. Dry, degassed dioxane was used in both chambers. The crude reaction mixture was purified via flash column chromatography on silica gel (heptane/DCM 5/5), furnishing the desired product as a yellow solid in 36% yield.

¹H NMR (300 MHz, CDCl₃): δ 8.19 (app. dd, J=8.1, 1.4 Hz, 1H), 7.61 (app. d, J=8.6 Hz, 2H), 7.43 (app. d, J=8.6 Hz, 2H), 7.32 (app. ddd, J=8.5, 7.1, 1.6 Hz, 1H), 6.72 (app. ddd, J=8.1, 7.1, 1.0 Hz, 1H), 6.66 (app. dd, J=8.4, 0.6 Hz, 1H), 6.36 (br, s, 2H), 1.34 (s, 9H). ¹³C NMR (101 MHz, CDCl₃): δ 179.63, 154.05, 151.04, 135.19, 134.48, 132.68, 125.65, 118.96, 117.46, 116.76, 116.08, 92.87, 86.90, 35.03, 31.07. IR (neat): ν 3426.99 (N—H stretch), 3317.44 (N—H stretch), 3075.61 (C—H stretch), 2959.86 (C—H stretch), 2920.59 (C—H stretch), 2850.31 (C—H stretch), 2186.82 (C≡C stretch), 1616.35 (C═O stretch). HR-MS (ESI): m/z calculated for [M+H]⁺ 278.1545, found 278.1539. MP: Tₘ 74.1-75.2.

### 3-(4-(tert-butyl)phenyl)-1-(pyridin-3-yl)prop-2-yn-1-one (1.15)

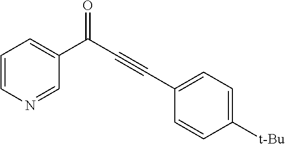 [SMILES: CC(C)(C)c1ccc(C#CC(=O)c2cccnc2)cc1]

General procedure A for the synthesis of propynones was followed using 3-bromopyridine (50 μL) and 1-(tert-butyl)-4-ethynylbenzene (141 μL) at 80° C. Dry, degassed dioxane was used in both chambers. The crude reaction mixture was purified via flash column chromatography on silica gel (DCM to DCM/MeOH 99/1), yielding 24% of the title compound as a brown oil.

¹H NMR (300 MHz, CDCl₃): δ 9.46 (app. dd, J=2.2, 0.8 Hz, 1H), 8.84 (app. dd, J=4.8, 1.7 Hz, 1H), 8.44 (app. dt, J=8.0, 2.0 Hz, 1H), 7.65 (app. d, J=8.6 Hz, 2H), 7.48 (app. dd, J=7.9, 0.9 Hz, 1H), 7.47 (app. d, J=8.6 Hz, 2H), 1.35 (s, 9H). ¹³C NMR (101 MHz, CDCl₃): δ 176.63, 155.34, 154.27, 151.64, 136.33, 133.38, 132.51, 126.06, 123.69, 116.62, 95.69, 86.36, 35.34, 31.19. IR (neat): ν 3040.47 (C—H stretch), 2957.79 (C—H stretch), 2926.79 (C—H stretch), 2852.38 (C—H stretch), 2195.09 (C≡C stretch), 1641.15 (C═O stretch). HR-MS (ESI): m/z calculated for [M+H]⁺ 264.1388, found 264.1392.

### 3-(4-(tert-butyl)phenyl)-1-phenylprop-2-yn-1-one (1.16)

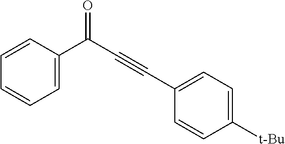 [SMILES: CC(C)(C)c1ccc(C#CC(=O)c2ccccc2)cc1]

General procedure A for the synthesis of propynones was followed using bromobenzene (1.07 mL, 10.00 mmol, 1.00 equiv.) and 1-(tert-butyl)-4-ethynylbenzene (2.82 mL, 15.00 mmol, 1.50 equiv.) at 80° C. The quantities of the other reagents and solvents were adapted accordingly. Dry, degassed dioxane was used in both chambers. The crude reaction mixture was purified via flash column chromatography on silica gel (heptane/DCM 8/2), yielding 80% of the title compound as a yellow solid.

¹H NMR (400 MHz, CDCl₃): δ 8.26-8.20 (m, 2H), 7.65-7.58 (m, 3H), 7.54-7.48 (m, 2H), 7.46-7.41 (m, 2H), 1.33 (s, 9H). ¹³C NMR (101 MHz, CDCl₃): δ 178.07, 154.58, 137.01, 134.00, 132.99, 129.54, 128.59, 125.76, 117.03, 93.80, 86.75, 35.09, 31.05. IR (neat): ν 3038.40 (C—H stretch), 2963.99 (C—H stretch), 2904.05 (C—H stretch), 2866.85 (C—H stretch), 2190.96 (C≡C stretch), 1634.95 (C═O stretch). HR-MS (ESI): m/z calculated for [M+H]⁺ 263.1436, found 263.1431. MP: Tₘ 49.2-50.2.

All data are in accordance with Wu et al. 2010 cited above and Liu et al. Org. Lett. 2008, 10 (18), 3933-3936.

### 1-(2-aminopyridin-3-yl)-3-(3-(trifluoromethyl)phenyl)prop-2-yn-1-one (1.17)

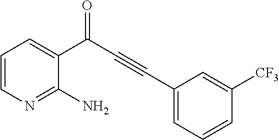 [SMILES: Nc1ncccc1C(=O)C#Cc1cccc(C(F)(F)F)c1]

General procedure A for the synthesis of propynones was followed using 3-bromopyridin-2-amine (88 mg) and 1-ethynyl-3-(trifluoromethyl)benzene (111 μL) at 80° C. Dry, degassed dioxane was used in both chambers. The crude reaction mixture was purified via column chromatography on silica gel (DCM/Et₂O 9/1), furnishing the desired product as a bright yellow solid in 26% yield.

¹H NMR (400 MHz, CDCl₃): δ 8.42 (app. dd, J=7.9, 1.9 Hz, 1H), 8.31 (app. dd, J=4.7, 1.9 Hz, 1H), 7.91 (app. s, 1H), 7.83 (app. d, J=7.8 Hz, 1H), 7.73 (app. d, J=7.9 Hz, 1H), 7.57 (app. dd, J=7.9, 7.8 Hz, 1H), 6.75 (app. dd, J=7.9, 4.7 Hz, 1H). ¹³C NMR (101 MHz, CDCl₃): δ 177.89, 159.16, 155.50, 143.21, 135.99, 131.62 (q, J=33.1 Hz), 129.65 (q, J=3.8 Hz), 129.51, 127.34 (q, J=3.7 Hz), 123.53 (q, J=272.7 Hz), 121.30, 114.10, 113.06, 91.11, 87.18. IR (neat): ν 3414.59 (N—H stretch), 3269.90 (N—H stretch), 3108.68 (C—H stretch), 2918.52 (C—H stretch), 2201.29 (C≡C stretch), 1612.21 (C═O stretch). HR-MS (ESI): m/z calculated for [M+H]+ 291.0745, found 291.0736. MP: Tₘ 168.3-168.9.

### 3-(4-(tert-butyl)phenyl)-1-(2-methoxyphenyl)prop-2-yn-1-one (1.18)

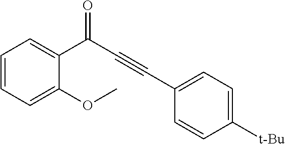 [SMILES: COc1ccccc1C(=O)C#Cc1ccc(C(C)(C)C)cc1]

General procedure A for the synthesis of propynones was followed using 1-bromo-2-methoxybenzene (1.28 mL, 10.00 mmol, 1.00 equiv.) and 1-(tert-butyl)-4-ethynylbenzene (2.82 mL, 15.00 mmol, 1.50 equiv.) at 80° C. The quantities of the other reagents and solvents were adapted accordingly. Dry, degassed dioxane was used in both chambers. The crude reaction mixture was purified via column chromatography on silica gel (heptane/DCM 4/6), yielding 9% of the title compound as a dark brown oil.

¹H NMR (600 MHz, CDCl₃): δ 8.08 (app. dd, J=7.9, 1.6 Hz, 1H), 7.58 (app. d, J=8.3 Hz, 2H), 7.54 (app. ddd, J=8.4, 7.4, 1.6 Hz, 1H), 7.42 (app. d, J=8.3 Hz, 2H), 7.05 (app. ddd, J=7.9, 7.4 Hz, 1H), 7.02 (app. d, J=8.4 Hz, 1H), 3.97 (s, 3H), 1.33 (s, 9H). ¹³C NMR (151 MHz, CDCl₃): δ 176.90, 159.79, 154.18, 134.87, 132.91, 132.64, 126.96, 125.67, 120.31, 117.64, 112.21, 92.29, 89.05, 55.95, 35.07, 31.08. IR (neat): ν 3071.47 (C—H stretch), 2961.93 (C—H stretch), 2866.85 (C—H stretch), 2837.91 (C—H stretch), 2195.09 (C≡C stretch), 1618.41 (C═O stretch). HR-MS (ESI): m/z calculated for [M+H]⁺ 293.1542, found 293.1538.

### 3-(4-(tert-butyl)phenyl)-1-(2-hydroxyphenyl)prop-2-yn-1-one (1.19)

Protocol based on Ma et al. Org. Lett. 2016, 18 (6), 1322-1325.

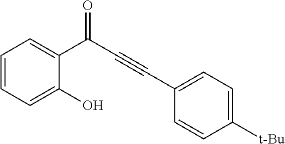 [SMILES: CC(C)(C)c1ccc(C#CC(=O)c2ccccc2O)cc1]

In a round-bottom flask, rac-2-(3-(4-(tert-butyl)phenyl)-1-hydroxyprop-2-yn-1-yl)phenol (3.2, 800 mg, 2.85 mmol, 1.00 equiv.) was stirred in the presence of manganese dioxide (1.736 g, 19.97 mmol, 7.00 equiv.) for 3 h at room temperature. The suspension was filtered over Celite® 535 and the filtrate was concentrated under reduced pressure. The residue was purified via column chromatography (heptane/EtOAc 95/5). The title compound was obtained as a bright yellow solid in 22% yield.

¹H NMR (600 MHz, CDCl₃): δ 11.79 (s, 1H), 8.13 (app. dd, J=7.9, 1.3 Hz, 1H), 7.64 (app. d, J=8.4 Hz, 2H), 7.53 (app. ddd, J=8.8, 7.2, 1.7 Hz, 1H), 7.46 (app. d, J=8.4 Hz, 2H), 7.02-6.98 (m, 2H), 1.35 (s, 9H). ¹³C NMR (151 MHz, CDCl₃): δ 182.51, 162.94, 155.21, 137.16, 133.22, 133.17, 126.00, 121.00, 119.50, 118.26, 116.74, 96.94, 85.72, 35.31, 31.18. IR (neat): ν 3046.67 (C—H stretch), 2957.79 (C—H stretch), 2924.72 (C—H stretch), 2868.91 (C—H stretch), 2197.16 (C≡C stretch), 1618.41 (C═O stretch). HR-MS (ESI): m/z calculated for [M+H]⁺ 279.1385, found 279.1378. MP: Tₘ 131.1-136.7 (decomposition).

### 3-(3-chlorophenyl)-1-(2-chloropyridin-3-yl)prop-2-yn-1-one (1.20)

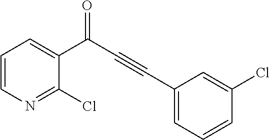 [SMILES: O=C(C#Cc1cccc(Cl)c1)c1cccnc1Cl]

General procedure B for the synthesis of propynones was followed using 2-chloronicotinaldehyde (283 mg, 2.00 mmol, 1.00 equiv.) and 1-chloro-3-ethynylbenzene (0.74 mL, 6.00 mmol, 3.00 equiv.). The quantities of the other reagents and solvents were adapted accordingly. The crude reaction mixture after the oxidation step was purified via flash column chromatography on silica gel (heptane/EtOAc 8/2). The title compound was obtained as an off-white solid in 83% overall yield.

¹H NMR (400 MHz, CDCl₃): δ 8.63-8.53 (m, 1H), 8.37-8.29 (m, 1H), 7.65 (app. s, 1H), 7.59-7.52 (m, 1H), 7.52-7.46 (m, 1H), 7.46-7.41 (m, 1H), 7.38 (app. t, J=7.8 Hz, 1H). ¹³C NMR (101 MHz, CDCl₃): δ 175.34, 152.66, 149.67, 140.84, 134.85, 132.87, 132.48, 131.68, 131.37, 130.18, 122.59, 121.43, 93.47, 88.36. IR (neat): ν 3119.01 (C—H stretch), 3065.27 (C—H stretch), 2203.36 (C≡C stretch), 1632.88 (C═O stretch). IR (neat): ν 3119.01 (C—H stretch), 3065.27 (C—H stretch), 2203.36 (C≡C stretch), 1632.88 (C═O stretch). HR-MS (ESI): m/z calculated for [M+H]⁺ 275.9983, found 275.9979. MP: Tₘ 114.1-114.8.

### 3-(3-chlorophenyl)-1-(2-morpholinopyridin-3-yl)prop-2-yn-1-one (1.21)

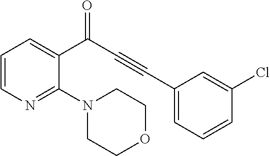 [SMILES: O=C(C#Cc1cccc(Cl)c1)c1cccnc1N1CCOCC1]

General procedure B for the synthesis of propynones was followed using 2-morpholinonicotinaldehyde (384 mg, 2.00 mmol) and 1-chloro-3-ethynylbenzene (0.74 mL, 6.00 mmol). The quantities of the other reagents and solvents were adapted accordingly. The crude reaction mixture after the oxidation step was purified via flash column chromatography on silica gel (heptane/EtOAc 75/25). The purified product was obtained as a yellow liquid, which could be solidified into a bright yellow solid by crash precipitation in n-pentane. The title compound was obtained in 92% overall yield.

¹H NMR (400 MHz, CDCl₃): δ 8.39-8.33 (m, 2H), 7.61 (app. s, 1H), 7.55-7.50 (m, 1H), 7.48-7.43 (m, 1H), 7.39-7.33 (m, 1H), 6.85 (m, 1H), 3.84 (br, app. t, J=4.3 Hz, 4H), 3.52 (br, app. t, J=4.3 Hz, 4H). ¹³C NMR (101 MHz, CDCl₃): δ 176.40, 159.15, 152.32, 143.04, 134.80, 132.72, 131.14, 131.12, 130.14, 122.01, 119.51, 114.43, 89.84, 88.42, 66.94, 50.20. IR (neat): ν 3065.27 (C—H stretch), 2970.19 (C—H stretch), 2856.51 (C—H stretch), 2197.16 (C≡C stretch), 1628.75 (C═O stretch). HR-MS (ESI): m/z calculated for [M+H]⁺ 327.0900, found 327.0891. MP: Tₘ 87.6-88.5.

### 1-(2-aminopyridin-3-yl)-3-(4-(tert-butyl)phenyl)prop-2-yn-1-one (1.22)

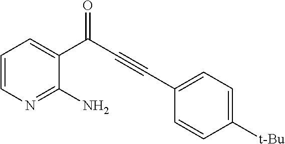 [SMILES: CC(C)(C)c1ccc(C#CC(=O)c2cccnc2N)cc1]

General procedure A for the synthesis of propynones was followed using 3-bromopyridin-2-amine (88 mg) and 1-(tert-butyl)-4-ethynylbenzene (181 μL, 1.00 mmol, 2.00 equiv.) at 100° C. In both chambers, 2 mL of solvent was used. The crude reaction mixture was purified via flash column chromatography on silica gel (heptane/EtOAc 95/5 to 6/4), yielding 64% of the desired product as a bright yellow solid.

¹H NMR (400 MHz, CDCl₃): δ 8.45 (app. d, J=7.8 Hz, 1H), 8.26 (app. d, J=3.2 Hz, 1H), 7.60 (app. d, J=8.2 Hz, 2H), 7.43 (app. d, J=8.2 Hz, 2H), 6.72 (app. dd, J=7.8, 3.2 Hz, 1H), 1.33 (s, 9H). ¹³C NMR (101 MHz, CDCl₃): δ 178.43, 159.16, 154.74, 154.63, 143.38, 132.89, 125.87, 117.06, 114.44, 112.75, 94.27, 86.27, 35.20, 31.16. IR (neat): ν 3389.78 (N—H stretch), 3263.70 (N—H stretch), 3133.48 (C—H stretch), 3038.40 (C—H stretch), 2959.86 (C—H stretch), 2866.85 (C—H stretch), 2186.82 (C≡C stretch), 1614.28 (C═O stretch). HR-MS (ESI): m/z calculated for [M+H]⁺ 279.1497, found 279.1494. MP: Tₘ 164.1-164.9.

### 1-(2-aminopyridin-3-yl)non-2-yn-1-one (1.23)

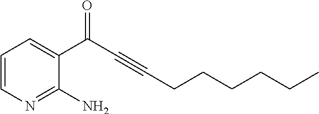 [SMILES: CCCCCCC#CC(=O)c1cccnc1N]

General procedure A for the synthesis of propynones was followed using 3-bromopyridin-2-amine (88 mg) and oct-1-yne (147 μL, 1.00 mmol, 2.00 equiv.) at 100° C. In both chambers, 2 mL of solvent was used. The crude reaction mixture was purified via flash column chromatography on silica gel (heptane/EtOAc 9/1 to 8/2), yielding 49% of the desired product as a brown solid.

¹H NMR (400 MHz, CDCl₃) δ 8.35 (app. dd, J=7.9, 1.4 Hz, 1H), 8.23 (app. dd, J=4.7, 1.4 Hz, 1H), 6.67 (app. dd, J=7.9, 4.7 1H), 2.48 (t, J=7.1 Hz, 2H), 1.65 (app. quint, J=7.4 Hz, 2H), 1.45 (app. quint, J=7.1 Hz, 2H), 1.31 (m, 4H), 0.90 (t, J=7.1 Hz, 3H). ¹³C NMR (101 MHz, CDCl₃): δ 178.66, 159.15, 154.34, 143.69, 114.44, 112.59, 97.33, 79.25, 31.33, 28.77, 27.89, 22.60, 19.31, 14.12. IR (neat): ν 3420.79 (N—H stretch), 3305.04 (N—H stretch), 2953.66 (C—H stretch), 2918.52 (C—H stretch), 2852.38 (C—H stretch), 2213.69 (C≡C stretch), 1620.48 (C═O stretch). HR-MS (ESI): m/z calculated for [M+H]⁺ 231.1497, found 231.1494. MP: Tₘ 75.8-77.6.

### 1-(3-aminopyridin-2-yl)-3-(4-(tert-butyl)phenyl)prop-2-yn-1-one (1.24)

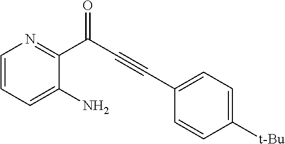 [SMILES: CC(C)(C)c1ccc(C#CC(=O)c2ncccc2N)cc1]

General procedure A for the synthesis of propynones was followed using 2-bromopyridin-3-amine (88 mg) and 1-(tert-butyl)-4-ethynylbenzene (181 μL, 1.00 mmol, 2.00 equiv.) at 100° C. In both chambers, 2 mL of solvent was used. The crude reaction mixture was purified via flash column chromatography on silica gel (heptane/EtOAc 8/2), furnishing the title compound as a light brown solid in 25% yield.

¹H NMR (400 MHz, CDCl₃): δ 8.02 (m, 1H), 7.51 (app. d, J=8.3 Hz, 2H), 7.38 (app. d, J=8.3 Hz, 2H), 7.02 (m, 2H), 4.31 (br, s, 2H), 1.32 (s, 9H). ¹³C NMR (101 MHz, CDCl₃): δ 152.34, 144.40, 140.08, 132.00, 131.66, 128.85, 125.58, 125.54, 123.79, 121.10, 119.41, 95.20, 84.67, 34.94, 31.23. IR (neat): ν 3360.85 (N—H stretch), 3048.74 (C—H stretch), 2959.86 (C—H stretch), 2901.99 (C—H stretch), 2866.85 (C—H stretch), 2215.76 (C≡C stretch), 1686.62 (C═O stretch). MP: Tₘ 125.8-127.2.

### 3-(3-chlorophenyl)-1-(pyridin-3-yl)prop-2-yn-1-one (1.25)

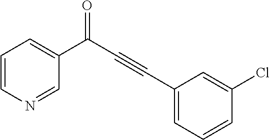 [SMILES: O=C(C#Cc1cccc(Cl)c1)c1cccnc1]

General procedure B for the synthesis of propynones was followed using nicotinaldehyde (93 μL) and 1-chloro-3-ethynylbenzene (0.37 mL). The crude reaction mixture after the oxidation step was purified via flash column chromatography on silica gel (heptane/acetone 97/3). The purified product was obtained as a dark yellow solid in 99% overall yield.

¹H NMR (400 MHz, CDCl₃): δ 9.42 (app. s, 1H), 8.86 (app. dd, J=4.7, 1.1 Hz, 1H), 8.42 (app. dt, J=7.8, 1.6 Hz, 1H), 7.7-7.66 (m, 1H), 7.61-7.56 (m, 1H), 7.52-7.45 (m, 2H), 7.42-7.35 (m, 1H). ¹³C NMR (101 MHz, CDCl₃): δ 176.15, 154.46, 151.43, 136.22, 134.77, 132.83, 132.02, 131.49, 131.27, 130.10, 123.61, 121.29, 92.42, 86.69. IR (neat): ν 3048.74 (C—H stretch), 2961.93 (C—H stretch), 2201.29 (C≡C stretch), 1632.88 (C═O stretch). HR-MS (ESI): m/z calculated for [M+H]⁺ 242.0373, found 242.0369. MP: Tₘ 106.2-106.6.

All data are in accordance with CN102256487

### 3-(3-chlorophenyl)-1-(1H-imidazol-2-yl)prop-2-yn-1-one (1.26)

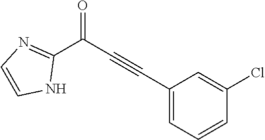 [SMILES: O=C(C#Cc1cccc(Cl)c1)c1ncc[nH]1]

General procedure B for the synthesis of propynones was followed using 1H-imidazole-2-carbaldehyde (96 mg) and 1-chloro-3-ethynylbenzene (0.37 mL). The crude reaction mixture after the oxidation step was purified via flash column chromatography on silica gel (heptane/EtOAc 5/5). The purified product was obtained as a beige solid in 53% overall yield.

¹H NMR (400 MHz, DMSO-d₆): δ 13.71 (br, s, 1H), 7.82-7.77 (m, 1H), 7.72-7.63 (m, 2H), 7.59 (br s, 1H), 7.57-7.51 (m, 1H), 7.31 (br, s, 1H). ¹³C NMR (101 MHz, DMSO-d₆): δ 166.35, 145.63, 133.63, 132.26, 132.00, 131.49, 131.30, 131.02, 123.30, 121.25, 89.89, 87.71. IR (neat): ν 3121.08 (C—H stretch), 2966.06 (C—H stretch), 2850.31 (C—H stretch), 2199.23 (C≡C stretch), 1622.55 (C═O stretch). HR-MS (ESI): m/z calculated for [M+H]⁺ 231.0325, found 231.0322. MP: Tₘ 170.1-170.8.

### 3-(3-chlorophenyl)-1-(2-(dimethylamino)pyridin-3-yl)prop-2-yn-1-one (1.27)

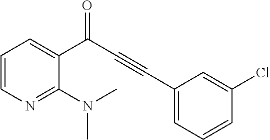 [SMILES: CN(C)c1ncccc1C(=O)C#Cc1cccc(Cl)c1]

General procedure B for the synthesis of propynones was followed using 2-(dimethylamino)nicotinaldehyde (151 mg) and 1-chloro-3-ethynylbenzene (0.37 mL). The crude reaction mixture after the oxidation step was purified via flash column chromatography on silica gel (heptane/EtOAc 95/5). The purified product was obtained as a dark yellow solid in 91% overall yield.

¹H NMR (400 MHz, CDCl₃): δ 8.37 (app. dd, J=7.5, 1.8 Hz, 1H), 8.32 (app. dd, J=4.7, 1.8 Hz, 1H), 7.62 (app. s, 1H), 7.55-7.49 (m, 1H), 7.46-7.41 (m, 1H), 7.35 (app. t, J=7.8 Hz, 1H), 6.72 (app. dd, J=7.9, 4.6 Hz, 1H), 3.08 (s, 6H). ¹³C NMR (101 MHz, CDCl₃): δ 175.18, 158.43, 151.88, 142.46, 134.51, 132.44, 130.90, 130.71, 129.93, 122.07, 117.12, 111.84, 89.21, 88.31, 41.40. IR (neat): ν 3063.21 (C—H stretch), 2922.65 (C—H stretch), 2850.31 (C—H stretch), 2199.23 (C≡C stretch), 1614.28 (C═O stretch). HR-MS (ESI): m/z calculated for [M+H]⁺ 285.0795, found 285.0794. MP: Tₘ 49.4-52.1.

### 1,3-diphenylprop-2-yn-1-one (1.28)

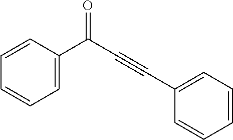 [SMILES: O=C(C#Cc1ccccc1)c1ccccc1]

General procedure A for the synthesis of propynones was followed using bromobenzene (266 μL, 2.50 mmol, 1.00 equiv.) and ethynylbenzene (0.41 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 9/1 to 7/3), furnishing the title compound as a yellow solid in 76% yield.

¹H NMR (400 MHz, CDCl₃): δ 8.26-8.20 (m, 2H), 7.72-7.67 (m, 2H), 7.64 (app. t, J=7.4 Hz, 1H), 7.56-7.46 (m, 3H), 7.43 (app. t, J=7.3 Hz, 2H). ¹³C NMR (101 MHz, CDCl₃): δ 178.05, 136.93, 134.14, 133.10, 130.82, 129.61, 128.72, 128.65, 120.17, 93.13, 86.92. IR (neat): ν 3050.81 (C—H stretch), 2195.09 (C≡C stretch), 1630.82 (C═O stretch). LR-MS (ESI): m/z calculated for [M+H]⁺ 207.0810, found 207.1. MP: Tₘ 48.4-48.9 (literature 46-48).

All data are in accordance with Wu et al. 2010 cited above, Liu et al. 2008 cited above, Iman et al. Synthesis 1990, 1990 (07), 631-632, Chen et al, Tetrahedron 2009, 65 (49), 10134-10141 and Zhang et al. Org. Biomol. Chem. 2020, 18 (6), 1073-1077.

### 3-(4-methoxyphenyl)-1-phenylprop-2-yn-1-one (1.29)

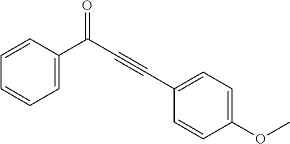 [SMILES: COc1ccc(C#CC(=O)c2ccccc2)cc1]

General procedure A for the synthesis of propynones was followed using bromobenzene (266 μL, 2.50 mmol, 1.00 equiv.) and 1-ethynyl-4-methoxybenzene (0.49 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 9/1 to 3/7) and subsequent recrystallization (heptane/i-PrOH 8/2), furnishing the title compound as a yellow solid in 83% yield.

¹H NMR (400 MHz, CDCl₃): δ 8.25-8.19 (m, 2H), 7.68-7.59 (m, 3H), 7.52 (app. t, J=7.6 Hz, 2H), 6.94 (app. d, J=8.8 Hz, 2H), 3.87 (s, 3H). ¹³C NMR (101 MHz, CDCl₃): δ 178.21, 161.90, 137.24, 135.31, 134.05, 129.66, 128.72, 114.59, 112.10, 94.46, 87.04, 55.61. IR (neat): ν 3052.87 (C—H stretch), 3013.60 (C—H stretch), 2842.04 (C—H stretch), 2182.69 (C≡C stretch), 1624.62 (C═O stretch). LR-MS (ESI): m/z calculated for [M+Na]⁺ 259.0735, found 259.1. MP: Tₘ 81.5-82.6 (literature 80-82).

All data are in accordance with with Wu et al. 2010 cited above, Liu et al. 2008 cited above, Iman et al. 1990 cited above, Chen et al 2009 cited above, Zhang et al. Org. Biomol. Chem. 2020, 18 (6), 1073-1077, Bishop et al. Synthesis 2004, 2004 (01), 43-52, Jeong et al. J. Org. Chem. 2014, 79 (14), 6444-6455, and Zhang et al. Org. Biomol. Chem. 2014, 12 (47), 9702-9706.

### 1-phenyl-3-(p-tolyl)prop-2-yn-1-one (1.30)

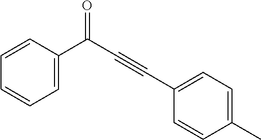 [SMILES: Cc1ccc(C#CC(=O)c2ccccc2)cc1]

General procedure A for the synthesis of propynones was followed using bromobenzene (266 μL, 2.50 mmol, 1.00 equiv.) and 1-ethynyl-4-methylbenzene (0.48 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 8/2 to 5/5) and subsequent recrystallization (heptane), furnishing the title compound as a yellow solid in 64% yield.

¹H NMR (400 MHz, CDCl₃): δ 8.26-8.19 (m, 2H), 7.67-7.56 (m, 3H), 7.52 (app. t, J=7.6 Hz, 2H), 7.23 (app. d, J=7.9 Hz, 2H), 2.41 (s, 3H). ¹³C NMR (101 MHz, CDCl₃): δ 178.23, 141.71, 137.45, 134.15, 133.28, 129.70, 129.64, 128.74, 117.18, 93.96, 86.94, 21.92. IR (neat): ν 3023.93 (C—H stretch), 2916.45 (C—H stretch), 2850.31 (C—H stretch), 2193.02 (C≡C stretch), 1626.68 (C═O stretch). LR-MS (ESI): m/z calculated for [M+Na]⁺ 243.0786, found 243.1. MP: Tₘ 59.8-60.6 (literature 67-71).

All data are in accordance with Wu et al. 2010 cited above, Liu et al. 2008 cited above, Chen et al 2009 cited above, Bishop et al. 2004 cited above, Jeon et al. 2014 cited above and Zhang et al. 2014 cited above.

### 3-(4-chlorophenyl)-1-phenylprop-2-yn-1-one (1.31)

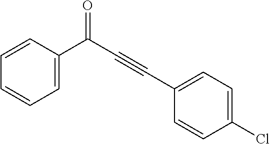 [SMILES: O=C(C#Cc1ccc(Cl)cc1)c1ccccc1]

General procedure A for the synthesis of propynones was followed using bromobenzene (266 μL, 2.50 mmol, 1.00 equiv.) and 1-chloro-4-ethynylbenzene (512 mg, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 8/2 to 6/4) and subsequent recrystallization (heptane/i-PrOH 8/2), furnishing the title compound as a brown solid in 49% yield.

¹H NMR (400 MHz, CDCl₃): δ 8.25-8.17 (m, 2H), 7.69-7.59 (m, 3H), 7.53 (app. t, J=7.7 Hz, 2H), 7.42 (app. d, J=8.5 Hz, 2H). ¹³C NMR (101 MHz, CDCl₃): δ 177.98, 137.37, 136.91, 134.42, 134.41, 129.74, 129.34, 128.84, 118.77, 91.76, 87.75. IR (neat): ν 3085.94 (C—H stretch), 3065.27 (C—H stretch), 3057.01 (C—H stretch), 2197.16 (C≡C stretch), 1632.88 (C═O stretch). HR-MS (ESI): m/z calculated for [M+H]⁺ 241.0420, found 241.0423. MP: Tₘ 104.2-105.8 (literature 105-108).

All data are in accordance with Chen et al. 2009 cited above and Bishop et al. 2004 cited above.

### 3-(3-methoxyphenyl)-1-phenylprop-2-yn-1-one (1.32)

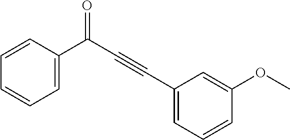 [SMILES: COc1cccc(C#CC(=O)c2ccccc2)c1]

General procedure A for the synthesis of propynones was followed using bromobenzene (266 μL, 2.50 mmol, 1.00 equiv.) and 1-ethynyl-3-methoxybenzene (0.45 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 7/3 to 3/7), furnishing the title compound as a yellow solid in 55% yield.

¹H NMR (400 MHz, CDCl₃): δ 8.27-8.19 (m, 2H), 7.64 (app. t, J=7.4 Hz, 1H), 7.53 (app. t, J=7.7 Hz, 2H), 7.37-7.27 (m, 2H), 7.20 (app. s, 1H), 7.07-7.01 (m, 1H), 3.85 (s, 3H). ¹³C NMR (101 MHz, CDCl₃): δ 178.17, 159.63, 137.04, 134.30, 129.95, 129.75, 128.79, 125.73, 121.23, 117.75, 117.71, 93.16, 86.73, 55.59. IR (neat): ν 3011.53 (C—H stretch), 2961.93 (C—H stretch), 2935.06 (C—H stretch), 2839.98 (C—H stretch), 2190.96 (C≡C stretch), 1632.88 (C═O stretch). HR-MS (ESI): m/z calculated for [M+H]⁺ 237.0916, found 237.0911. MP: Tₘ 73.9-74.8.

### 1-phenyl-3-(m-tolyl)prop-2-yn-1-one (1.33)

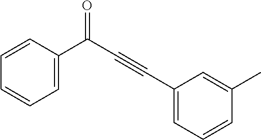 [SMILES: Cc1cccc(C#CC(=O)c2ccccc2)c1]

General procedure A for the synthesis of propynones was followed using bromobenzene (266 μL, 2.50 mmol, 1.00 equiv.) and 1-ethynyl-3-methylbenzene (0.48 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 8/2 to 5/5), furnishing the title compound as a brown solid in 55% yield.

¹H NMR (400 MHz, CDCl₃): δ 8.23 (app. d, J=7.4 Hz, 2H), 7.63 (app. t, J=7.2 Hz, 1H), 7.56-7.47 (m, 4H), 7.34-7.27 (m, 2H), 2.39 (s, 3H). ¹³C NMR (101 MHz, CDCl₃): δ 178.21, 138.68, 137.11, 134.21, 133.71, 131.89, 130.38, 129.72, 128.76, 128.74, 120.09, 93.65, 86.82, 21.34. IR (neat): ν 3061.14 (C—H stretch), 3032.20 (C—H stretch), 2945.39 (C—H stretch), 2916.45 (C—H stretch), 2188.89 (C≡C stretch), 1630.82 (C═O stretch). LR-MS (ESI): m/z calculated for [M+H]⁺ 221.0966, found 221.1. MP: Tₘ 30.8-31.8.

All data are in accordance with Zhang et al. 2020 cited above.

### 3-(3-chlorophenyl)-1-phenylprop-2-yn-1-one (1.34)

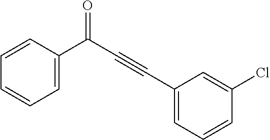 [SMILES: O=C(C#Cc1cccc(Cl)c1)c1ccccc1]

General procedure A for the synthesis of propynones was followed using bromobenzene (266 μL, 2.50 mmol, 1.00 equiv.) and 1-chloro-3-ethynylbenzene (0.46 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 7/3), furnishing the title compound as a beige solid in 29% yield.

¹H NMR (600 MHz, CDCl₃): δ 8.26-8.20 (m, 2H), 7.71-7.65 (m, 2H), 7.59 (app. d, J=7.6 Hz, 1H), 7.55 (app. t, J=7.7 Hz, 2H), 7.51-7.46 (m, 1H), 7.39 (app. t, J=7.9 Hz, 1H). ¹³C NMR (151 MHz, CDCl₃): δ 177.84, 136.81, 134.76, 134.47, 132.79, 131.25, 131.16, 130.11, 129.73, 128.84, 122.00, 91.02, 87.51. IR (neat): ν 3065.27 (C—H stretch), 2201.29 (C≡C stretch), 1630.82 (C═O stretch). LR-MS (ESI): m/z calculated for [M+H]⁺ 241.0420, found 241.0. MP: Tₘ 75.1-75.4.

All data are in accordance with Jeong et al. 2014 cited above.

### 3-(3-fluorophenyl)-1-phenylprop-2-yn-1-one (1.35)

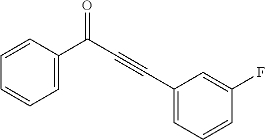 [SMILES: O=C(C#Cc1cccc(F)c1)c1ccccc1]

General procedure A for the synthesis of propynones was followed using bromobenzene (266 μL, 2.50 mmol, 1.00 equiv.) and 1-ethynyl-3-fluorobenzene (0.43 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 8/2 to 6/4), furnishing the title compound as a yellow solid in 20% yield.

¹H NMR (600 MHz, CDCl₃): δ 8.24 (app. d, J=7.2 Hz, 2H), 7.67 (app. t, J=7.4 Hz, 1H), 7.56 (app. t, J=7.7 Hz, 2H), 7.50 (app. d, J=7.6 Hz, 1H), 7.46-7.38 (m, 2H), 7.23 (app. td, J=8.4, 1.6 Hz, 1H). ¹³C NMR (151 MHz, CDCl₃): δ 177.78, 162.33 (d, J=248.1 Hz), 136.72, 134.34, 130.45 (d, J=8.5 Hz), 129.62, 128.95 (d, J=3.2 Hz), 128.72, 121.97 (d, J=9.3 Hz), 119.69 (d, J=23.2 Hz), 118.25 (d, J=21.2 Hz), 91.08 (d, J=3.5 Hz), 87.14. IR (neat): ν 3061.14 (C—H stretch), 2195.09 (C≡C stretch), 1649.42 (C═O stretch). LR-MS (ESI): m/z calculated for [M+Na]⁺ 247.0535, found 247.1. MP: Tₘ 59.3-59.8 (literature 48-61.0).

All data are in accordance with Zhang et al. 2014 cited above and Yilmaz et al. Chemistry Select 2019, 4 (37), 11043-11047.

### 3-(2-methoxyphenyl)-1-phenylprop-2-yn-1-one (1.36)

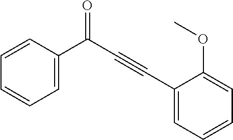 [SMILES: COc1ccccc1C#CC(=O)c1ccccc1]

General procedure A for the synthesis of propynones was followed using bromobenzene (266 μL, 2.50 mmol, 1.00 equiv.) and 1-ethynyl-2-methoxybenzene (0.49 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 7/3 to 3/7), furnishing the title compound as a yellow solid in 56% yield.

¹H NMR (600 MHz, CDCl₃): δ 8.36-8.33 (m, 2H), 7.67-7.61 (m, 2H), 7.54 (app. t, J=7.7 Hz, 2H), 7.50-7.45 (m, 1H), 7.01 (app. t, J=7.5 Hz, 1H), 6.98 (app. d, J=8.4 Hz, 1H), 4.00 (s, 3H). ¹³C NMR (151 MHz, CDCl₃): δ 178.14, 161.90, 137.19, 135.03, 133.87, 132.63, 129.78, 128.53, 120.71, 110.87, 109.48, 91.26, 90.55, 55.94. IR (neat): ν 3065.27 (C—H stretch), 2986.73 (C—H stretch), 2941.26 (C—H stretch), 2835.84 (C—H stretch), 2197.16 (C≡C stretch), 1643.22 (C═O stretch). HR-MS (ESI): m/z calculated for [M+H]⁺ 237.0916, found 237.0908. MP: Tₘ 73.1-74.2 (literature 74-75).

All data are in accordance with Chen et al. 2009 and Iman et al. 1990 cited above.

### 1-phenyl-3-(o-tolyl)prop-2-yn-1-one (1.37)

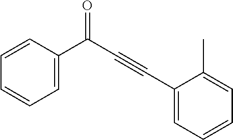 [SMILES: Cc1ccccc1C#CC(=O)c1ccccc1]

General procedure A for the synthesis of propynones was followed using bromobenzene (266 μL, 2.50 mmol, 1.00 equiv.) and 1-ethynyl-2-methylbenzene (0.47 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 8/2 to 5/5), furnishing the title compound as a brown solid in 52% yield.

¹H NMR (400 MHz, CDCl₃): δ 8.27-8.21 (m, 2H), 7.68-7.60 (m, 2H), 7.52 (app. t, J=7.6 Hz, 2H), 7.41-7.35 (m, 1H), 7.29 (app. d, J=7.2 Hz, 1H), 7.27-7.20 (m, 1H), 2.59 (s, 3H). ¹³C NMR (101 MHz, CDCl₃): δ 178.08, 142.19, 137.08, 134.05, 133.68, 130.82, 129.90, 129.56, 128.65, 125.96, 120.04, 92.19, 90.78, 20.91. IR (neat): ν 3063.21 (C—H stretch), 2963.99 (C—H stretch), 2918.52 (C—H stretch), 2850.31 (C—H stretch), 2186.82 (C≡C stretch), 1634.95 (C═O stretch). HR-MS (ESI): m/z calculated for [M+H]⁺ 221.0966, found 221.0968. MP: Tₘ 37.8-39.0.

All data are in accordance with Liu et al. 2008 cited above and Chen et al. 2009 cited above.

### 3-(2-chlorophenyl)-1-phenylprop-2-yn-1-one (1.38)

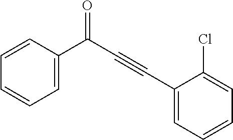 [SMILES: O=C(C#Cc1ccccc1Cl)c1ccccc1]

General procedure A for the synthesis of propynones was followed using bromobenzene (266 μL, 2.50 mmol, 1.00 equiv.) and 1-chloro-2-ethynylbenzene (0.46 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 7/3), furnishing the title compound as a dark yellow solid in 81% yield.

¹H NMR (600 MHz, CDCl₃): δ 8.36-8.31 (m, 2H), 7.78-7.72 (m, 1H), 7.67 (app. t, J=7.4 Hz, 1H), 7.59-7.51 (m, 3H), 7.45 (app. dd, J=7.5, 1.4 Hz, 1H), 7.38-7.33 (app. dd, J=7.5, 1.0 Hz, 1H). ¹³C NMR (151 MHz, CDCl₃): δ 177.86, 137.55, 136.83, 135.11, 134.26, 131.79, 129.83, 129.69, 128.69, 126.87, 120.51, 91.06, 89.02. IR (neat): ν 3059.07 (C—H stretch), 2199.23 (C≡C stretch), 1632.88 (C═O stretch). HR-MS (ESI): m/z calculated for [M+H]⁺ 241.0420, found 241.0417. MP: Tₘ 94.1-94.8 (literature 94).

All data are in accordance with Iman et al. 1990 cited above.

### 3-(4-(tert-butyl)phenyl)-1-(3-fluorophenyl)prop-2-yn-1-one (1.39)

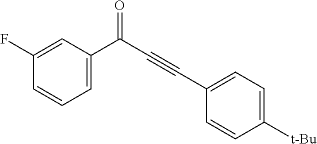 [SMILES: CC(C)(C)c1ccc(C#CC(=O)c2cccc(F)c2)cc1]

General procedure A for the synthesis of propynones was followed using 1-bromo-3-fluorobenzene (218 μL, 2.50 mmol, 1.00 equiv.) and 1-chloro-2-ethynylbenzene (0.68 mL, 3.75 mmol, 1.50 equiv.) at 80° C. The quantities of the other reagents and solvents were adapted accordingly. Dry, degassed dioxane was used in both chambers. The crude reaction mixture was purified via column chromatography on silica gel (petroleum ether/DCM 9/1 to 7/3), furnishing the title compound as a yellow solid in 78% yield.

¹H NMR (600 MHz, CDCl₃): δ 8.05 (app. d, J=7.7 Hz, 1H), 7.91 (app. d, J=9.1 Hz, 1H), 7.66 (app. d, J=8.4 Hz, 2H), 7.56-7.50 (m, 1H), 7.48 (app. d, J=8.4 Hz, 2H), 7.35 (app. td, J=8.2, 1.7 Hz, 1H), 1.37 (s, 9H). ¹³C NMR (151 MHz, CDCl₃): δ 176.68 (d, J=2.6 Hz), 162.77 (d, J=248.2 Hz), 154.92, 139.10 (d, J=6.6 Hz), 133.11, 130.30 (d, J=7.7 Hz), 125.85, 125.41 (d, J=2.9 Hz), 121.03 (d, J=21.6 Hz), 116.72, 115.97 (d, J=22.7 Hz), 94.49, 86.48, 35.16, 31.06. IR (neat): ν 3054.94 (C—H stretch), 2957.79 (C—H stretch), 2904.05 (C—H stretch), 2866.85 (C—H stretch), 2211.63 (C≡C stretch), 1639.08 (C═O stretch). HR-MS (ESI): m/z calculated for [M+H]⁺ 281.1342, found 281.1335. MP: Tₘ 53.7-54.7.

### 1-(2-aminophenyl)-3-(3-chlorophenyl)prop-2-yn-1-one (1.40)

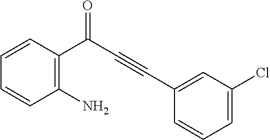 [SMILES: Nc1ccccc1C(=O)C#Cc1cccc(Cl)c1]

General procedure B for the synthesis of propynones was followed using freshly prepared 2-aminobenzaldehyde (363 mg, 3.00 mmol, 1.00 equiv.) and 5.00 equivalents of 1-chloro-3-ethynylbenzene (1.84 mL, 15.00 mmol, 5.00 equiv.) 2-aminobenzaldehyde was prepared via oxidation of 2-aminobenzyl alcohol. Therefore, a flame-dried round-bottom flask was charged with 2-aminobenzyl alcohol (370 mg, 3.00 mmol, 1.00 equiv.) and manganese dioxide (1.49 g, 21.0 mmol, 7.00 equiv.). The flask was closed with a septum, purged with N2 and dry DCM (30 mL) was added via syringe through the septum. The reaction mixture was stirred for 45 min at ambient temperature. Afterwards, the mixture was filtered over a pad of Celite® 535 and the filtrate was concentrated under reduced pressure. The crude product was used without further purification. The quantities of the other reagents and solvents were adapted accordingly. The crude reaction mixture after the oxidation step was purified via flash column chromatography on silica gel (heptane/EtOAc 95/5). Spectroscopic analysis of the product fraction indicated the presence of some residual aldehyde. Therefore, the mixture was dissolved in DMF (10 mL) and saturated aqueous NaHSO₃ (25 mL) was added to the solution. The mixture was shaken thoroughly for half a minute. Afterwards, the mixture was diluted with water and extracted three times with a 9/1 mixture of EtOAc/hexane (25 mL). The combined organic layers were washed three times with water, dried over Na₂SO₄, filtered and concentrated by rotary evaporation. This extraction procedure was repeated three times. The purified product was obtained as an orange solid in 12% overall yield.

¹H NMR (400 MHz, CDCl₃): δ 8.13 (app. dd, J=8.3, 1.2 Hz, 1H), 7.64 (app. t, 1.6 Hz, 1H), 7.54 (app. dt, J=7.6, 1.3 Hz, 1H), 7.44 (app. ddd, J=8.1, 2.1, 1.2 Hz, 1H), 7.37-7.34 (m, 1H), 7.34-7.30 (m, 1H), 6.73 (app. t, J=7.3 Hz, 1H), 6.67 (app. dt, J=8.3, 0.5 Hz, 1H), 3.67 (br s, 2H). ¹³C NMR (101 MHz, CDCl₃): δ 179.05, 151.18, 135.52, 134.54, 134.39, 132.42, 130.87, 130.62, 129.89, 122.34, 118.74, 116.82, 116.22, 90.21, 87.69. IR (neat): ν 3447.66 (N—H stretch), 3313.31 (N—H stretch), 3065.27 (C—H stretch), 2955.73 (C—H stretch), 2922.65 (C—H stretch), 2852.38 (C—H stretch), 2201.29 (C≡C stretch), 1616.35 (C═O stretch). HR-MS (ESI): m/z calculated for [M+H]⁺ 256.0529, found 256.0529. MP: Tₘ 225.2-228.9 (decomposition).

### 1-(2-chlorophenyl)-3-(3-chlorophenyl)prop-2-yn-1-one (1.41)

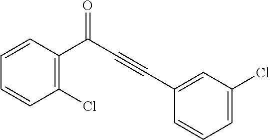 [SMILES: O=C(C#Cc1cccc(Cl)c1)c1ccccc1Cl]

General procedure B for the synthesis of propynones was followed using 2-chlorobenzaldehyde (0.11 mL) and 5.00 equivalents of 1-chloro-3-ethynylbenzene (1.84 mL, 15.00 mmol, 5.00 equiv.). The crude reaction mixture after the oxidation step was purified via flash column chromatography on silica gel (heptane/EtOAc 98/2), furnishing the title compound as an off-white solid in 98% yield.

¹H NMR (400 MHz, CDCl₃): δ 8.06 (app. dt, J=7.6, 0.9 Hz, 1H), 7.62 (app. t, J=1.6 Hz, 1H), 7.53 (app. dt, J=7.6, 1.1 Hz, 1H), 7.49 (app. d, J=4.1 Hz, 2H), 7.48-7.44 (m, 1H), 7.44-7.38 (m, 1H), 7.35 (app. t, J=7.8 Hz, 1H). ¹³C NMR (101 MHz, CDCl₃): δ 176.47, 135.64, 134.66, 133.67, 133.60, 132.67, 132.55, 131.62, 131.19, 131.15, 129.96, 126.88, 121.81, 91.73, 88.71. IR (neat): ν 3061.14 (C—H stretch), 2953.66 (C—H stretch), 2920.59 (C—H stretch), 2852.38 (C—H stretch), 2203.36 (C≡C stretch), 1637.02 (C═O stretch). HR-MS (ESI): m/z calculated for [M+H]⁺ 275.0030, found 275.0027. MP: Tₘ 70.5-71.8 (literature 77-79).

All data are in accordance with Shao et al. Synthesis 2012, 44 (12), 1798-1805.

### Example 17

### Synthesis of Diarylketones

### (4-methoxyphenyl)(phenyl)methanone (II.1)

Protocol based on Veryser et al., React. Chem. Eng. 2016, 1(2), 142-146, and Ahlburg et al. J. Org. Chem. 2013, 78(20), 10310-10318.

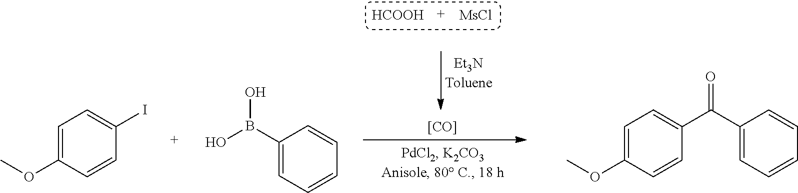

To the right chamber of a flame-dried two-chamber reactor (COware) were added 1-iodo-4-methoxybenzene (117 mg, 0.50 mmol, 1.00 equiv.), phenylboronic acid (73 mg, 0.75 mmol, 1.5 equiv.), potassium carbonate (207 mg, 1.50 mmol, 3.00 equiv.), and palladium(II) chloride (0.9 mg, 0.005 mmol, 1.0 mol %). The reactor was closed with two screw caps and septa and was evacuated and backfilled with argon three times. Dry, degassed toluene (2 mL) was added to the left chamber, followed by formic acid (29 μL, 0.75 mmol, 1.50 equiv.) and mesylchloride (58 μL, 0.75 mmol, 1.50 equiv.). To the right chamber, dry, degassed anisole (2 mL) was added. The reaction was initiated by the addition of triethylamine (0.21 mL, 1.50 mmol, 3.00 equiv.) to the left chamber of the reactor. Immediately after the addition of the triethylamine, the reactor was placed in an oil bath at 80° C. for 18 h. When the reaction was finished, the crude reaction mixture was filtered over a pad of Celite® 535. The filtrate was concentrated in vacuo and purified via flash column chromatography on silica gel (heptane/EtOAc 9/1), furnishing the title compound as pale yellow oil in 66% yield.

¹H NMR (300 MHz, CDCl₃): δ 7.86-7.79 (m, 2H), 7.78-7.63 (m, 2H), 7.60-7.53 (m, 1H), 7.51-7.43 (m, 2H), 7.00-6.94 (m, 2H), 3.89 (s, 3H). ¹³C NMR (75 MHz, CDCl₃): δ 195.60, 163.23, 138.29, 132.58, 131.91, 130.16, 129.75, 128.20, 113.56, 55.51. IR (neat): ν 3057.01 (C—H stretch), 3003.27 (C—H stretch), 2932.99 (C—H stretch), 2839.98 C—H stretch), 1649.42 (C═O stretch). LR-MS (ESI): m/z calculated for [M+Na]⁺ 235.0735, found 235.2. MP: Tₘ 52.5-53.2 (literature 49.7-59).

All data are in accordance with Ahlburg et al. 2013 cited above, Jin et al. Synlett 2011, 2011 (10), 1435-1438, Cho et al. Catal. Commun. 2008, 9 (13), 2261-2263 and Wang et al. Synth. Commun. 2001, 31 (24), 3885-3890.

### Example 18

### Synthesis of Propynals

### 3-(4-(tert-butyl)phenyl)propiolaldehyde (2.1)

Protocol based on Bugarin et al. Tetrahedron Lett. 2015, 56 (23), 3285-3287.

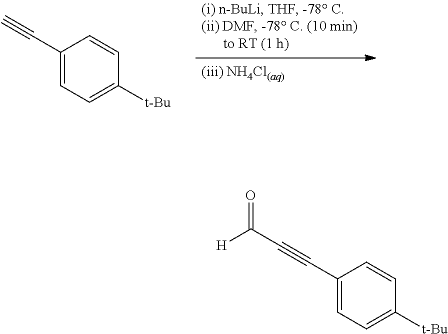

To a solution of the 1-(tert-butyl)-4-ethynylbenzene (2.71 mL, 15.00 mmol, 1.00 equiv.) in dry THF (6 mL) was added n-BuLi (2.5 M in hexanes, 6.00 mL, 15.00 mmol, 1.00 equiv.) at −78° C., while stirring under argon. To this solution was added dry DMF (5.81 mL, 75.00 mmol, 5.00 equiv.) dropwise over 10 min at the same temperature. After the addition, the solution was allowed to warm up to room temperature. After 1 h of further stirring, the solution was quenched with saturated aqueous NH₄Cl and extracted with DCM. The organic layers were combined, washed sequentially with water and brine, dried over MgSO₄ and filtered. The filtrate was concentrated under reduced pressure and purified via column chromatography on silica gel (heptane to heptane/Et₂O 98/2), yielding 5% of the desired product as a pale yellow oil.

¹H NMR (400 MHz, CDCl₃): δ 9.41 (s, 1H), 7.55 (app. d, J=8.6 Hz, 2H), 7.43 (app. d, J=8.6 Hz, 2H), 1.33 (s, 9H). ¹³C NMR (101 MHz, CDCl₃): δ 176.96, 155.32, 133.37, 125.96, 116.45, 96.05, 88.56, 35.28, 31.14. IR (neat): ν 3052.87 (C—H stretch), 2953.66 (C—H stretch), 2922.65 (C—H stretch), 2873.05 (C—H stretch), 2854.45 (C—H stretch aldehyde), 2730.43 (C—H stretch aldehyde), 2188.89 (C≡C stretch), 1665.95 (C═O stretch). HR-MS (ESI): m/z calculated for [M+H]⁺ 187.1123, found 187.1101.

All data are in accordance with Murata et al. Chem. Commun. 2020, 56 (43), 5783-5786.

### Example 19

### Synthesis of Propynols

### rac-3-(4-(tert-butyl)phenyl)-1-(2-methoxyphenyl)prop-2-yn-1-ol (3.1)

Protocol based on Jeong, et al. J. Org. Chem. 2014, 79 (14), 6444-6455.

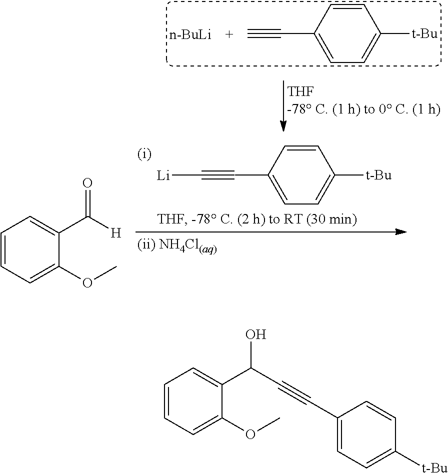

To a flame-dried two-neck round-bottom flask, equipped with a septum and a nitrogen balloon, were added 1-(tert-butyl)-4-ethynylbenzene (3.25 mL, 18.00 mmol, 1.20 equiv.) and dry THF (45 mL). The solution was cooled to −78° C., and n-BuLi (2.5 M in hexanes, 7.50 mL, 18.75 mmol, 1.25 equiv.) was added slowly via a syringe through the septum. The mixture was stirred at −78° C. for 1 h, then warmed to 0° C., stirred for 1 h, and then re-cooled to −78° C. A solution of 2-methoxybenzaldehyde (2.042 g, 15.00 mmol, 1.00 equiv.) in dry THF (15 mL) was added dropwise via a syringe and the reaction was stirred at −78° C. for 1 h, warmed to room temperature and stirred for an additional 30 min. Next, the reaction was quenched with saturated aqueous NH₄Cl and extracted with Et₂O. The combined organic layers were washed with brine, dried over MgSO₄, filtered, and concentrated by rotary evaporation. The residue was purified via flash column chromatography on silica gel (heptane/EtOAc 9/1), yielding 64% of the title compound as a pale, viscous liquid.

¹H NMR (600 MHz, CDCl₃): δ 7.65 (app. dd, J=7.5, 1.4 Hz, 1H), 7.42 (app. d, J=8.5 Hz, 2H), 7.33 (app. d, J=8.5 Hz, 2H), 7.35 (app. ddd, J=8.0, 7.5, 1.4 Hz, 1H), 7.00 (app. ddd, J=7.5, 7.5, 0.8 Hz, 1H), 6.94 (app. dd, J=8.0, 0.8 Hz, 1H), 5.93 (d, J=6.2 Hz, 1H), 3.93 (s, 3H), 3.03 (d, J=6.2 Hz, 1H), 1.31 (s, 9H). ¹³C NMR (101 MHz, CDCl₃): δ 157.00, 151.74, 131.60, 129.80, 129.07, 128.19, 125.34, 121.00, 119.84, 111.00, 87.77, 86.38, 61.77, 55.73, 34.86, 31.27. IR (neat): ν 3416.65 (O—H stretch), 3036.34 (C—H stretch), 2959.86 (C—H stretch), 2866.85 (C—H stretch), 2837.91 (C—H stretch), 2195.09 (C≡C stretch). HR-MS (ESI): m/z calculated for [M+Na]⁺ 317.1517, found 317.1515.

### rac-2-(3-(4-(tert-butyl)phenyl)-1-hydroxyprop-2-yn-1-yl)phenol (3.2)

Protocol based on Ma et al. 2016, cited above.

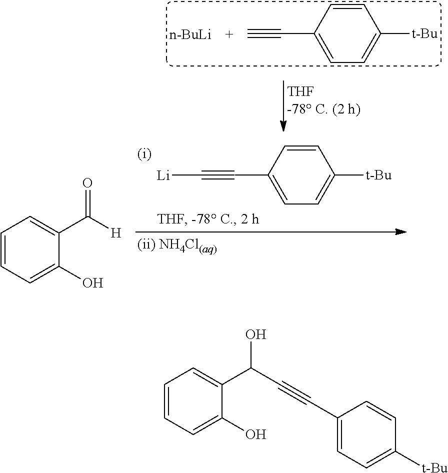

To a solution of 1-(tert-butyl)-4-ethynylbenzene (1.99 mL, 11.00 mmol, 2.20 equiv.) in dry THF (35 mL), n-BuLi (2.5 M in hexanes, 4.20 mL, 10.50 mmol, 2.10 equiv.) was slowly added at −78° C. under nitrogen atmosphere. After 2 h, salicaldehyde (522 μL, 5.00 mmol, 1.00 equiv.) was added dropwise to the solution. The reaction mixture was stirred at −78° C. for another 2 h and was then quenched with saturated aqueous NH₄Cl. The mixture was extracted with DCM and the organic layers were combined, washed with brine, dried over Na₂SO₄ and filtered. The filtrate was concentrated under reduced pressure and purified via flash column chromatography on silica gel (heptane/EtOAc 8/2). The title compound was obtained as burgundy red solid in 47% yield.

¹H NMR (600 MHz, CDCl₃): δ 7.47-7.41 (m, 3H), 7.40-7.33 (m, 4H), 7.30-7.23 (m, 2H), 6.95-6.89 (m, 2H), 5.92 (br, s, 1H), 2.77 (d, J=3.6 Hz, 1H), 1.32 (s, 9H). ¹³C NMR (101 MHz, CDCl₃): δ 155.50, 152.45, 131.73, 130.32, 127.92, 125.54, 124.73, 120.38, 119.02, 117.29, 88.67, 85.95, 64.64, 34.97, 31.28. IR (neat): ν 3342.24 (O—H stretch), 3034.27 (C—H stretch), 2959.86 (C—H stretch), 2901.99 (C—H stretch), 2866.85 (C—H stretch), 2195.09 (C≡C stretch). HR-MS (ESI): m/z calculated for [M+H]⁺ 281.1542, found 281.1496. MP: Tₘ 131.1-136.7 (decomposition).

### rac-3-(4-(tert-butyl)phenyl)-1-phenylprop-2-yn-1-ol (3.3)

Protocol based on Jeong et al. J. Org. Chem. 2014, 79 (14), 6444-6455.

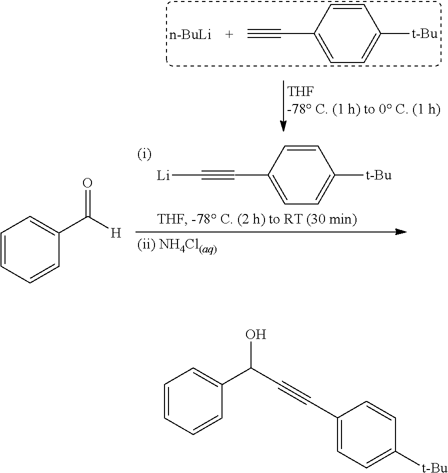

To a flame-dried two-neck round-bottom flask, equipped with a septum and a nitrogen balloon, were added 1-(tert-butyl)-4-ethynylbenzene (11.28 mL, 60.00 mmol, 1.20 equiv.) and dry THF (180 mL). The solution was cooled to −78° C., and n-BuLi (2.5 M in hexanes, 25.00 mL, 62.50 mmol, 1.25 equiv.) was added slowly via a syringe through the septum. The mixture was stirred at −78° C. for 1 h, then warmed to 0° C., stirred for 1 h, and then re-cooled to −78° C. A solution of benzaldehyde (5.31 mL, 50.00 mmol, 1.00 equiv.) in dry THF (60 mL) was added dropwise via a syringe and the reaction was stirred at −78° C. for 1 h, warmed to room temperature and stirred for an additional 30 min. Next, the reaction was quenched with saturated aqueous NH₄Cl and extracted with Et₂O. The combined organic layers were washed with brine, dried over MgSO₄, filtered, and concentrated by rotary evaporation. The residue was purified via column chromatography on silica gel (heptane/DCM 5/5 to 2/8), yielding 65% of the title compound as a light yellow solid.

¹H NMR (400 MHz, CDCl₃): δ 7.65-7.59 (m, 2H), 7.44-7.37 (m, 4H), 7.34-7.31 (m, 3H), 5.65 (d, J=6.5 Hz, 1H), 2.61 (d, J=6.5 Hz, 1H), 1.29 (s, 9H). ¹³C NMR (101 MHz, CDCl₃): δ 151.93, 140.85, 131.58, 128.68, 128.41, 126.85, 125.38, 119.48, 88.22, 86.87, 65.15, 34.84, 31.22. IR (neat): ν 3249.23 (O—H stretch), 3036.34 (C—H stretch), 2963.99 (C—H stretch), 2904.05 (C—H stretch), 2866.85 (C—H stretch), 2197.16 (C≡C stretch). LR-MS (ESI): m/z calculated for [M+Na]⁺ 287.1412, found 287.2. MP: Tₘ 55.5-56.4.

All data are in accordance with Oshimoto et al. Org. Biomol. Chem. 2019, 17(17), 4225-4229.

### rac-1-(2-aminopyridin-3-yl)-3-(3-chlorophenyl)prop-2-yn-1-ol (3.4)

Protocol based on WO2012065963.

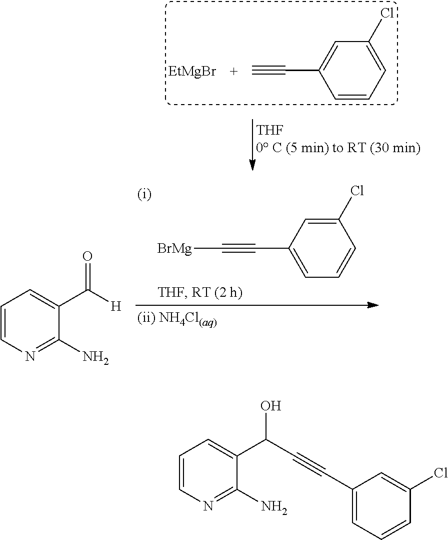

At 0° C., EtMgBr (3.0 M in Et₂O, 16.67 ml, 50.00 mmol, 5.00 equiv.) was added to a solution of 3-chloro-1-ethynylbenzene (6.16 mL, 50.0 mmol, 5.00 equiv.) in dry THF (100 mL) and stirred for 5 min at 0° C. and then for 30 min at room temperature. This solution was slowly added to a solution of 2-aminonicotinaldehyde (1.22 g, 10.0 mmol, 1.00 equiv.) in dry THF (80 mL) under a nitrogen atmosphere at room temperature. After 2 h, the mixture was quenched with saturated aqueous NH₄Cl, extracted with DCM and the organic layers were washed with brine and dried over Na₂SO₄. The resulting organic solution was filtered and the filtrate was concentrated under reduced pressure. The residue was purified via flash column chromatography on silica gel (heptane/EtOAc 65/35 to 5/5). The title compound was obtained as a yellow solid in 20% yield.

¹H NMR (400 MHz, CDCl₃): δ 7.99 (app. d, J=4.6 Hz, 1H), 7.71 (app. d, J=7.4 Hz, 1H), 7.45 (app. s, 1H), 7.30-7.27 (m, 3H), 6.67 (app. dd, J=7.3, 5.2 Hz, 1H), 5.64 (s, 1H), 5.19 (br, s, 2H), 3.65 (br, s, 1H). ¹³C NMR (101 MHz, CDCl₃): δ 157.41, 147.68, 135.37, 133.73, 131.27, 131.03, 130.55, 129.31, 124.55, 119.37, 112.72, 91.54, 83.64, 60.73. IR (neat): ν 3358.78 (N—H stretch), 3098.34 (O—H stretch), 2922.65 (C—H stretch), 2850.31 (C—H stretch), 2687.02 (C—H stretch), 2219.89 (C≡C stretch). HR-MS (ESI): m/z calculated for [M+H]⁺ 259.0638, found 259.0638. MP: Tₘ 138.8-140.1.

### Example 20

### Synthesis of Propynes

### 1-(tert-butyl)-4-(3-phenylprop-1-yn-1-yl)benzene (4.1)

Protocol based on Madu et al. Tetrahedron 2017, 73 (43), 6118-6137.

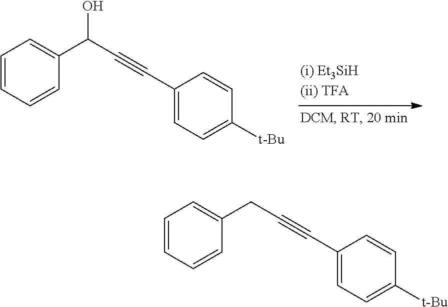

Triethylsilane (319 μL, 2.00 mmol, 2.00 equiv.) was added at room temperature to a solution of (4-(tert-butyl)phenyl)-1-phenylprop-2-yn-1-ol (3.3) (264 mg, 1.00 mmol, 1.00 equiv.) in dry DCM (2 ml) under nitrogen atmosphere. Then 2,2,2-trifluoroacetic acid (297 μL, 4.00 mmol, 4.00 equiv.) was added and the solution was stirred for 20 min. The reaction mixture was quenched with saturated aqueous NaHCO₃ and then extracted with DCM. The organic layers were combined, dried over MgSO₄ and filtered. The filtrate was concentrated in vacuo and purified via flash column chromatography on silica gel (heptane to heptane/DCM 95/5). The desired product was obtained as a yellow oil in 10% yield.

¹H NMR (400 MHz, CDCl₃): δ 7.45 (m, 9H), 3.83 (s, 2H), 1.31 (s, 9H). ¹³C NMR (101 MHz, CDCl₃): δ 151.15, 137.07, 131.49, 128.65, 128.10, 126.72, 125.37, 120.80, 86.85, 82.86, 34.85, 31.33, 25.90. IR (neat): ν 3061.14 (C—H stretch), 2961.93 (C—H stretch), 2904.05 (C—H stretch), 2868.91 (C—H stretch), 2197.16 (C≡C stretch).

### Example 21

### Synthesis of Propenones

### (E/Z)-3-(4-(tert-butyl)phenyl)-3-(ethylthio)-1-phenylprop-2-en-1-one (5.1)

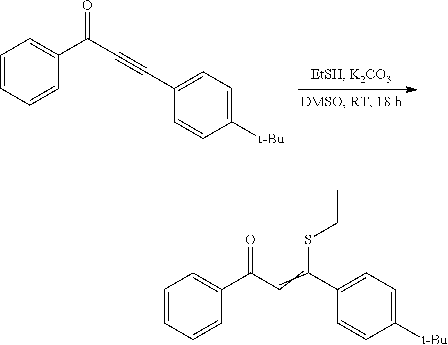 [SMILES: CC(C)(C)c1ccc(C#CC(=O)c2ccccc2)cc1 ; CCSC(=CC(=O)c1ccccc1)c1ccc(C(C)(C)C)cc1]

A round-bottom flask was charged with 3-(4-(tert-butyl)phenyl)-1-phenylprop-2-yn-1-one (1.16) (262 mg, 1.00 mmol, 1.00 equiv.), K₂CO₃ (415 mg, 3.00 mmol, 3.00 equiv.) and DMSO (10 mL) and closed with a septum. While stirring at room temperature, ethanethiol (0.74 mL, 10.00 mmol, 10.00 equiv.) was added to the closed reaction flask via a syringe through the septum. The reaction mixture was allowed to stir at room temperature for 18 h. After this time, the mixture was concentrated under reduced pressure and partitioned between DCM and a saturated aqueous solution of NaHCO₃. The organic phase was collected, dried over MgSO₄ and filtered. The filtrate was concentrated under reduced pressure and purified via flash column chromatography on silica gel (heptane/DCM 6/4). The desired product was obtained as a bright yellow solid in 89% yield as a mixture of two stereoisomers (E/Z ratio 22/78).

¹H NMR (400 MHz, CDCl₃): δ 7.98 (app. d, J=7.2 Hz, 2H, Z), 7.79 (app. d, J=7.2 Hz, 2H, E), 7.55-7.22 (m, 7H, E & Z), 7.06 (s, 1H, Z), 6.66 (s, 1H, E), 2.90 (q, J=7.4 Hz, 2H, E), 2.47 (q, J=7.5 Hz, 2H, Z), 1.39 (t, J=7.4 Hz, 3H, E), 1.36 (s, 9H, Z), 1.27 (s, 9H, E), 1.10 (t, J=7.5 Hz, 3H, Z). ¹³C NMR (101 MHz, CDCl₃): δ 189.29 (E), 188.40 (Z), 163.92 (Z), 160.21 (E), 152.15 (E), 152.10 (Z), 139.16 (E), 138.79 (Z), 136.32 (Z), 134.57 (E), 132.12 (Z), 132.00 (E), 128.48 (Z), 128.44 (E), 128.36 (E), 128.19 (E), 128.08 (Z), 127.77 (Z), 125.37 (Z), 125.04 (E), 119.57 (Z), 116.74 (E), 34.76 (Z), 34.67 (E), 31.31 (Z), 31.19 (E), 27.23 (Z), 27.04 (E), 14.20 (Z), 13.02 (E). IR (neat): ν 3065.27 (C═CH stretch), 2955.73 (C—H stretch), 2899.92 (C—H stretch), 2862.71 (C—H stretch), 1624.62 (C═O stretch). HR-MS (ESI): m/z calculated for [M+H]⁺ 325.1626, found 325.1617. MP: Tₘ 88.4-90.3.

### (E)-1-(2-aminopyridin-3-yl)-3-(3-chlorophenyl)prop-2-en-1-one (5.2)

Protocol based on Minders et al. Bioorg. Med. Chem. Lett. 2015, 25 (22), 5270-5276.

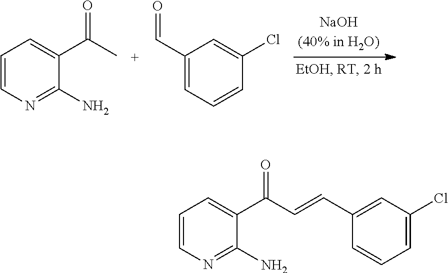

In a round bottom flask, 1-(2-aminopyridin-3-yl)ethan-1-one (70 mg, 0.50 mmol, 1.00 equiv.) and 3-chlorobenzaldehyde (72 mg, 0.50 mmol, 1.00 equiv.) were dissolved in absolute ethanol (1.4 mL). Subsequently, aqueous NaOH (40% in H₂O, 0.03 mL, 0.25 mmol, 0.50 equiv.) was added dropwise to the solution. After the addition, the mixture was stirred at room temperature for 2 h. Afterwards, the crude reaction mixture was extracted with EtOAc and washed with water. The organic layers were dried over Na₂SO₄, filtered and concentrated under reduced pressure. Crystallization from MeOH, followed by flash column chromatography on silica gel (heptane/EtOAc 6/4), furnished the title compound as a yellow solid in 57% yield.

¹H NMR (400 MHz, CDCl₃): δ 8.27 (app. d, J=4.4 Hz, 1H), 8.16 (app. d, J=7.8 Hz, 1H), 7.63 (ABq, Δν=55.3 Hz, J=15.6 Hz, 2H), 7.62 (app. s, 1H), 7.52-7.46 (m, 1H), 7.43-7.32 (m, 2H), 7.01 (br, s, 2H), 6.70 (app. dd, J=8.1, 4.8 Hz, 1H). ¹³C NMR (101 MHz, CDCl₃): δ 189.83, 159.62, 154.31, 142.19, 139.47, 136.76, 135.02, 130.28, 130.23, 127.79, 126.77, 122.88, 113.67, 112.36. IR (neat): ν 3381.52 (N—H stretch), 3265.77 (N—H stretch), 3199.63 (C—H stretch), 3150.02 (C—H stretch), 3059.07 (C—H stretch), 2924.72 (C—H stretch), 1649.42 (C═O stretch). HR-MS (ESI): m/z calculated for [M+H]⁺ 259.0638, found 259.0641. MP: Tₘ 122.4-123.7.

### Example 22

### Synthesis of Amides

### (2-aminopyridin-3-yl)(4-(3-chlorophenyl)piperazin-1-yl)methanone (6.1)

Protocol based on CN106279015.

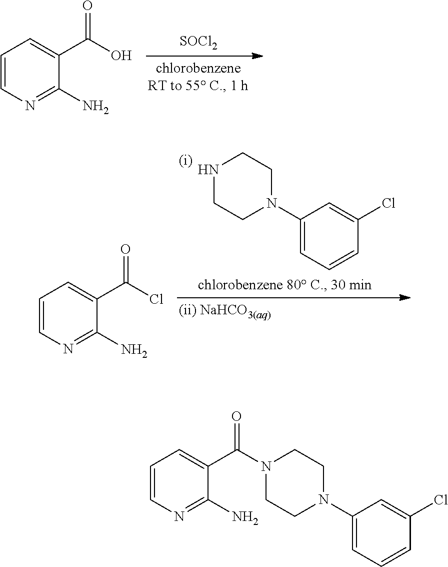 [SMILES: Nc1ncccc1C(=O)O ; "SOCl2" ; Nc1ncccc1C(=O)Cl ; Clc1cccc(N2CCNCC2)c1 ; Nc1ncccc1C(=O)N1CCN(c2cccc(Cl)c2)CC1]

To a solution of 2-aminonicotinic acid (414 mg, 3.00 mmol, 1.00 equiv.) in chlorobenzene (9 mL) was added dropwise thionyl chloride (1.09 mL, 15.00 mmol, 5.00 equiv.), while stirring at room temperature. After the addition was complete, the flask was heated to 55° C. for 1 h while stirring. After this period, the excess thionyl chloride was distilled off under reduced pressure. Subsequently, 1-(3-chlorophenyl)piperazine (1.48 mL, 9.00 mmol, 3.00 equiv.) was added to the flask and the solution was heated to 80° C. The reaction was stirred for 30 min at this temperature. After this period, the reaction was quenched with saturated aqueous NaHCO₃ and extracted with DCM. The organic layers were combined and washed with brine, dried over Na₂SO₄ and filtered. The filtrate was concentrated under reduced pressure. The residue was purified via column chromatography on silica gel (DCM/i-PrOH 95/5), yielding 22% of the title compound as an off-white solid.

¹H NMR (600 MHz, CDCl₃): δ 8.15 (app. dd, J=5.0, 1.8 Hz, 1H), 7.40 (app. dd, J=7.4, 1.8 Hz, 1H), 7.19 (app. t, J=8.0 Hz, 1H), 6.90-6.87 (m, 1H), 6.87-6.86 (m, 1H), 6.79 (app. ddd, J=8.4, 2.3, 0.7 Hz, 1H), 6.68 (app. dd, J=7.4, 5.0 Hz, 1H), 5.19 (br, s, 2H), 3.77 (br, s, 4H), 3.22 (br, t, J=5.0 Hz, 4H). ¹³C NMR (151 MHz, CDCl₃): δ 202.28, 168.84, 157.13, 154.26, 151.84, 150.18, 136.41, 135.11, 130.20, 120.39, 116.53, 114.56, 113.02, 49.36 (br). IR (neat): ν 3470.39 (N—H stretch), 3119.01 (C—H stretch), 2920.59 (C—H stretch), 2839.98 (C—H stretch), 1626.68 (C═O stretch). HR-MS (ESI): m/z calculated for [M+H]⁺ 317.1169, found 317.1159. MP: Tₘ 149.7-150.6.

### Example 23

### Synthesis of Quinolinones

### 2-phenylquinolin-4(1H)-one (III.1)

Protocol based on Akerbladh et al. J. Org. Chem. 2015, 80 (3), 1464-1471.

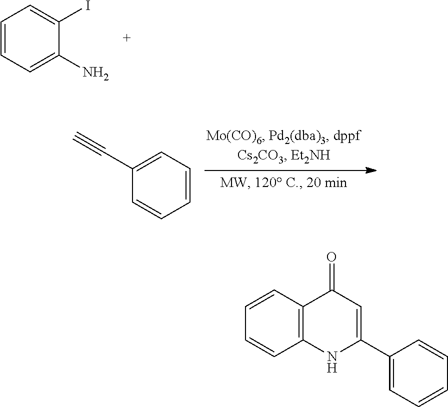

To a 0.5-2 mL microwave vial were added 2-iodoaniline (110 mg, 0.50 mmol, 1.00 equiv.), ethynylbenzene (110 μL, 1.00 mmol, 2.00 equiv.), tris(dibenzylideneacetone)dipalladium(0) (9.2 mg, 0.01 mmol, 2.0 mol %), 1,1′-bis(diphenylphosphino)ferrocene (11.2 mg, 0.02 mmol, 4.0 mol %),

molybdenum hexacarbonyl (132 mg, 0.50 mmol, 1.00 equiv), cesium carbonate (490 mg, 1.50 mmol, 3.00 equiv), and 1.5 mL of diethylamine. The vial was purged with nitrogen, capped with a snap-on cap, and irradiated in a microwave reactor at 120° C. for 20 min while stirring. Afterwards, the reaction mixture was diluted with water and extracted with DCM (3×15 mL). The organic layers were combined, dried over Na₂SO₄ and filtered. The filtrate was concentrated under reduced pressure and purified via flash column chromatography on silica gel (DCM/MeOH 99/1 to 95/5), furnishing the title compound as a light brown solid in 38% yield.

¹H NMR (400 MHz, DMSO-d₆): δ 11.70 (br, s, 1H), 8.10 (app. d, J=8.3 Hz, 1H), 7.87-7.80 (m, 2H), 7.80-7.74 (m, 1H), 7.70-7.63 (m, 1H), 7.63-7.56 (m, 3H), 7.37-7.31 (m, 1H), 6.33 (app. s, 1H). ¹³C NMR (101 MHz, DMSO-d₆): δ 176.93, 149.97, 140.52, 134.20, 131.73, 130.39, 128.95, 128.42, 127.39, 124.68, 123.21, 118.72, 107.31. IR (neat): ν 3061.14 (C═CH stretch), 2961.93 (C—H stretch), 2922.65 (C—H stretch), 1632.88 (C═O stretch), 1502.67 (C═C stretch). LR-MS (ESI): m/z calculated for [M+H]⁺ 222.0919, found 222.2. MP: Tₘ 210.2-212.1 (literature 252-254).

All data are in accordance with Huang et al. Org. Lett. 2008, 10 (12), 2609-2612, Kuo et al. J. Med. Chem. 1993, 36 (9), 1146-1156 and Lee & Youn Bull. Korean Chem. Soc. 2008, 29 (9), 1853-1856.

### 2-(4-(tert-butyl)phenyl)quinolin-4(1H)-one (7.1)

Protocol based on Akerbladh et al. J. Org. Chem. 2015, 80(3), 1464-1471.

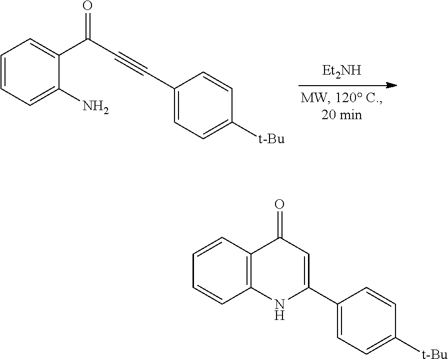 [SMILES: "Et2NH" ; CC(C)(C)c1ccc(C#CC(=O)c2ccccc2N)cc1 ; CC(C)(C)c1ccc(-c2cc(=O)c3ccccc3[nH]2)cc1]

To a 0.5-2 mL microwave vial were added 1-(2-aminophenyl)-3-(4-(tert-butyl)phenyl)prop-2-yn-1-one (1.14) (35.2 mg, 0.13 mmol, 1.00 equiv.) and Et₂NH (1.5 mL). The vial was purged with nitrogen, capped with a snap-on cap, and irradiated in a microwave reactor at 120° C. for 20 min while stirring. Afterwards, the reaction mixture was concentrated under reduced pressure. The residue was purified via flash column chromatography on silica gel (DCM to DCM/MeOH 9/1), furnishing the desired product as a white solid in 84% yield.

¹H NMR (400 MHz, DMSO-d₆): δ 11.67 (br, s, 1H), 8.10 (app. ddd, J=8.0, 1.5, 0.4 Hz, 1H), 7.78 (app. d, J=8.4 Hz, 2H), 7.75 (app. d, J=8.0 Hz, 1H), 7.66 (app. ddd, J=8.3, 6.9, 1.5 Hz, 1H), 7.61 (ap. d, J=8.5 Hz, 2H), 7.33 (app. ddd, J=7.2, 6.8, 0.8 Hz, 1H), 6.34 (s, 1H), 1.35 (s, 9H). ¹³C NMR (151 MHz, DMSO-d₆): δ 176.87, 153.21, 149.96, 140.54, 131.68, 131.46, 127.16, 125.77, 124.85, 124.68, 123.15, 118.68, 106.93, 34.59, 30.93. IR (neat): ν 3034.27 (C═CH stretch), 2954.39 (C—H stretch), 2899.92 (C—H stretch), 2866.85 (C—H stretch), 2771.77 (C—H stretch), 1632.88 (C═O stretch), 1496.47 (C═C stretch). HR-MS (ESI): m/z calculated for [M+H]⁺ 278.1545, found 278.1541. MP: Tₘ 322.2-322.8.

All data are in accordance with Wang et al. Synthesis 2017, 49 (18), 4309-4320 and Xu et al. Org. Lett. 2018, 20 (7), 1893-1897.

### Example 24

### Synthesis of Naphthyridinones

### 2-(4-(tert-butyl)phenyl)-1,8-naphthyridin-4(1H)-one (8.1)

Protocol based on Neumann et al 2014, Veryser et al. 2016 and Akerbladh et al. 2015, cited above.

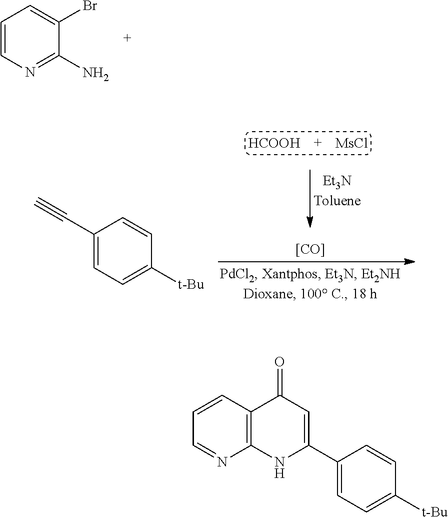

To the right chamber of a flame-dried two-chamber reactor (COWare) as added 3-bromopyridin-2-amine (86 mg, 0.50 mmol, 1.00 equiv.), palladium(II) chloride (4.4 mg, 0.025 mmol, 5.0 mol %) and Xantphos (14.5 mg, 0.025 mmol, 5.0 mol %). The reactor was closed with two screw caps and septa and was evacuated and backfilled with argon three times. Dry, degassed toluene (3 mL) was added to the left chamber, followed by formic acid (29 μL, 0.75 mmol, 1.50 equiv.) and mesylchloride (58 μL, 0.75 mmol, 1.50 equiv.). To the right chamber, dry, degassed dioxane (3 mL) was added, followed by 1-(tert-butyl)-4-ethynylbenzene (180 μL, 1.00 mmol, 2.00 equiv.), dry triethylamine (0.21 mL, 1.50 mmol, 3.00 equiv.) and dry diethylamine (0.16 mL, 1.50 mmol, 3.00 equiv.). The reaction was initiated by the addition of triethylamine (0.21 mL, 1.50 mmol, 3.00 equiv.) to the left chamber of the reactor. Immediately after the addition of the triethylamine, the reactor was placed in an oil bath at 100° C. for 18 h. When the reaction was finished, the crude reaction mixture was filtered over a pad of Celite® 535. The filtrate was concentrated in vacuo and purified via flash column chromatography on silica gel (DCM/MeOH 95/5 to 9/1), yielding 54% of the desired product as a beige solid.

¹H NMR (400 MHz, CDCl₃): δ 10.85 (br, 1H), 8.68 (app. dd, J=7.9, 1.9 Hz, 1H), 8.38 (app. dd, J=4.6, 1.9 Hz, 1H), 7.75-7.61 (m, 2H), 7.61-7.53 (m, 2H), 7.28 (app. dd, J=7.9, 4.6 Hz, 1H), 6.61 (s, 1H), 1.39 (s, 9H). ¹³C NMR (101 MHz, CDCl₃): δ 178.89, 154.65, 152.37, 151.54, 151.13, 136.25, 131.52, 127.13, 126.34, 120.36, 119.60, 109.58, 35.00, 31.22. IR (neat): ν 3036.34 (C═CH stretch), 2945.39 (C—H stretch), 2868.91 (C—H stretch), 1610.15 (C═O stretch). HR-MS (ESI): m/z calculated for [M+H]⁺ 279.1497, found 279.1494. MP: Tₘ 237.8-239.8.

### Example 25

### Synthesis of Thiopyranoxides

### 3-(4-(tert-butyl)phenyl)-1-methyl-5-phenyl-1λ⁶-thiopyran 1-oxide (9.1)

Protocol based on Hortmann & Harris J. Am. Chem. Soc. 1971, 93 (10), 2471-2481, Corey & Chaykovsky J. Am. Chem. Soc. 1965, 87 (6), 1353-1364 and Hortmann J. Am. Chem. Soc. 1965, 87 (21), 4972-4973.

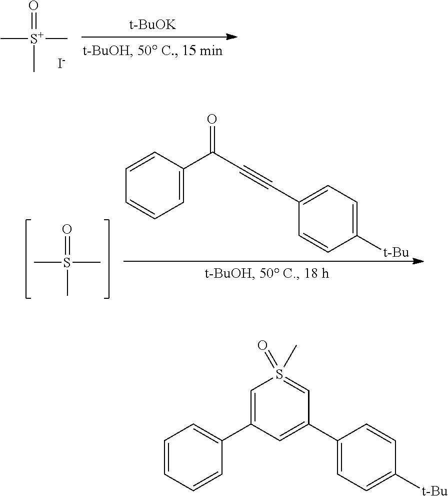

To a solution of trimethylsulfoxonium iodide (280 mg, 1.27 mmol, 3.18 equiv.) in tert-butanol (3 mL) was added potassium tert-butoxide (145 mg, 1.29 mmol, 3.23 equiv.), while stirring at 50° C. When the addition was complete, the mixture was stirred at the same temperature for another 15 min before a solution of 3-(4-(tert-butyl)phenyl)-1-phenylprop-2-yn-1-one (1.16) (105 mg, 0.40 mmol, 1.00 equiv.) in tert-butanol (2 mL) was added. The resultant mixture was stirred at 50° C. for 18 h. After this period, the reaction solvent was removed under reduced pressure. The residue was partitioned between DCM and water and the aqueous phase was extracted two more times with DCM. The combined organic layers were washed with brine and dried over Na₂SO₄. The filtrate was concentrated under reduced pressure and the residue was purified via flash column chromatography on silica gel (heptane/EtOAc 8/2 to 6/4). The title compound was obtained as a dark yellow solid in 79% yield.

¹H NMR (400 MHz, CDCl₃): δ 7.60 (app. d, J=7.0 Hz, 2H), 7.54 (app. d, J=8.3 Hz, 2H), 7.49-7.34 (m, 5H), 6.26 (app. s, 1H), 5.81 (ABq, Δν=7.8 Hz, J=4.2 Hz, 2H), 3.63 (s, 3H), 1.36 (s, 9H). ¹³C NMR (151 MHz, CDCl₃): δ 151.73, 145.97, 141.01, 137.97, 128.68, 128.48, 127.46, 127.14, 125.65, 102.01, 83.61, 83.38, 50.22, 34.66, 31.34. IR (neat): ν 3054.94 (C—H stretch), 2957.79 (C—H stretch), 2924.72 (C—H stretch), 2866.85 (C—H stretch), 1696.96 (C═S═O stretch, presumed). HR-MS (ESI): m/z calculated for [M+H]⁺ 337.1626, found 337.1613. MP: Tₘ 93.2-94.4.

### Example 26

### Synthesis of Pyrazolo[3,4-b]pyridines

### 3-((3-chlorophenyl)ethynyl)-1H-pyrazolo[3,4-b]pyridine (10.1)

Protocol based on Yadav et al., Chem. Eur. J. 2014, 20 (23), 7122-7127.

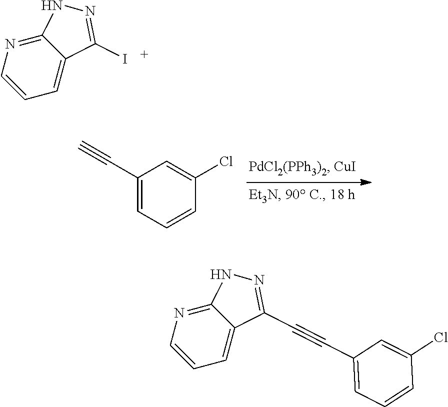

A flame-dried pressure tube was charged with 3-iodo-1H-pyrazolo[3-4-b]pyridine (250 mg, 1.00 mmol, 1.00 equiv.), copper(I) iodide (8 mg, 0.04 mmol, 4.0 mol %) and bis(triphenylphosphine)palladium(II) dichloride (28 mg, 0.04 mmol, 4.0 mol %). The tube was sealed with a screw cap and septum and evacuated and backfilled with N₂ three times. Next, dry triethylamine (4.4 mL) and 1-ethynyl-3-chlorobenzene (148 μL, 1.20 mmol, 1.20 equiv.) were added via a syringe through the septum. The reaction mixture was stirred at 90° C. for 18 h. Afterwards, the mixture was filtered over a pad of Celite® 535 and the filtrate was concentrated by rotary evaporation. The residue was purified via column chromatography on silica gel (heptane/EtOAc 8/2 to 6/4), followed by recrystallization from n-hexane at −78° C. The title compound was obtained as an off-white solid in 42% yield.

¹H NMR (600 MHz, CDCl₃): δ 12.80 (br, s, 1H), 8.70 (app. d, J=4.0 Hz, 1H), 8.28 (app. d, J=7.7 Hz, 1H), 7.64 (app. s, 1H), 7.53 (app. d, J=7.6 Hz, 1H), 7.40-7.36 (m, 1H), 7.35-7.33 (m, 1H), 7.33-7.29 (m, 1H). ¹³C NMR (101 MHz, DMSO-d₆): δ 151.93, 150.36, 133.90, 131.48, 131.19, 130.69, 129.84, 129.82, 127.36, 124.09, 118.47, 116.46, 91.40, 82.79. IR (neat): ν 3133.48 (C—H stretch), 3085.94 (C—H stretch), 2875.12 (C—H stretch), 2823.44 (C—H stretch), 2761.43 (C—H stretch), 2221.96 (C≡C stretch). HR-MS (ESI): m/z calculated for [M+H]⁺ 254.0485, found 254.0482. MP: Tₘ 210.1-211.0.

### 3-((3-chlorophenyl)ethynyl)-1-methyl-1H-pyrazolo[3,4-b]pyridine (10.2)

Protocol based on WO2012151158.

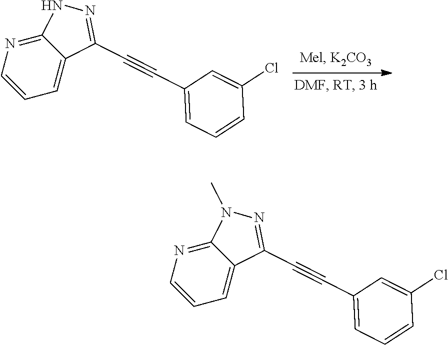

To a solution of 3-((3-chlorophenyl)ethynyl)-1H-pyrazolo[3,4-b]pyridine (10.1) (146 mg, 0.58 mmol, 1.00 equiv.) in dry DMF (11.5 mL) was added potassium carbonate (159 mg, 1.16 mmol, 2.00 equiv.) and methyliodide (39 μL, 0.63 mmol, 1.10 equiv.) at 0° C. The reaction mixture was slowly warmed to room temperature and stirred for an additional 3 h. Afterwards, the reaction mixture was diluted with water (50 mL) and extracted three times with EtOAc (50 mL). The combined organic layers were washed with brine, dried over Na₂SO₄ and filtered. The filtrate was concentrated under reduced pressure and purified via crystallization from MeOH. The desired product was obtained as an off-white solid in 98% yield.

¹H NMR (400 MHz, DMSO-d₆): δ 8.67 (app. dd, J=4.5, 1.5 Hz, 1H), 8.43 (app. dd, J=8.1, 1.5 Hz, 1H), 7.57-7.48 (m, 2H), 7.38 (app. dd, J=8.1, 4.5 Hz, 1H), 4.13 (s, 3H). ¹³C NMR (101 MHz, DMSO-d₆): δ 150.36, 150.12, 133.90, 131.43, 131.21, 130.67, 130.23, 129.86, 125.76, 124.02, 118.67, 117.02, 91.81, 82.37, 34.65. IR (neat): ν 2932.99 (C—H stretch), 2215.76 (C≡C stretch). HR-MS (ESI): m/z calculated for [M+H]⁺ 268.0641, found 268.0634. MP: Tₘ 136.0-136.3.

### Example 27

### Synthesis of Indazoles

### 3-((3-chlorophenyl)ethynyl)-1H-indazole (11.1)

Protocol based on Yadav et al. Chem. Eur. J. 2014, 20(23), 7122-7127.

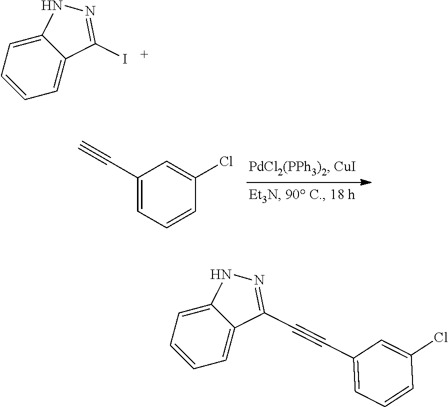

A flame-dried pressure tube was charged with 3-iodo-1H-indazole (244 mg, 1.00 mmol, 1.00 equiv.), copper(I) iodide (8 mg, 0.04 mmol, 4.0 mol %) and bis(triphenylphosphine)palladium(II) dichloride (28 mg, 0.04 mmol, 4.0 mol %). The tube was sealed with a screw cap and septum and evacuated and backfilled with N₂ three times. Next, dry triethylamine (4.4 mL) and 1-ethynyl-3-chlorobenzene (148 μL, 1.20 mmol, 1.20 equiv.) were added via a syringe through the septum. The reaction mixture was stirred at 90° C. for 18 h. Afterwards, the mixture was filtered over a pad of Celite® 535 and the filtrate was concentrated by rotary evaporation. The residue was purified via flash column chromatography on silica gel (heptane/EtOAc 8/2), followed by recrystallization from n-heptane. The title compound was obtained as a white solid in 21% yield.

¹H NMR (400 MHz, DMSO-d₆): δ 13.58 (s, 1H), 7.90 (app. d, J=8.1 Hz, 1H), 7.78 (app. s, 1H), 7.67-7.60 (m, 2H), 7.57-7.42 (m, 3H), 7.27 (app. t, J=7.5 Hz). ¹³C NMR (101 MHz, DMSO-d₆): δ 140.00, 133.41, 130.83, 130.68, 130.12, 129.09, 127.29, 126.96, 124.19, 123.97, 121.69, 119.68, 110.88, 90.83, 83.03. IR (neat): ν 3154.15 (C—H stretch), 3125.22 (C—H stretch), 2910.25 (C—H stretch), 2221.96 (C≡C stretch), 1593.61 (C═C stretch). HR-MS (ESI): m/z calculated for [M+H]⁺ 253.0533, found 253.0519. MP: Tₘ 198.1-198.8.

### Example 28

### Pharmacokinetic Analysis

Male NMRI mice (average body weight 30 g) were maintained as described in example 3. For each time period (i.e., 2 min, 15 min, 30 min, 1 h, 2-2.5 h, 4 h, 8 h, and 24 h), 1-5 mice were i.p. injected with 200 μL (injection volume was adjusted to the individual weight) of VHC (8% solutol/12% PEG200/80% water) or 300 mg/kg test compound dissolved in VHC. After the treatment period, blood samples were drawn from the tail veins, collected in Greiner MiniCollect K2EDTA tubes, and centrifuged twice for 5 min at 15,000 g to obtain plasma samples. Three volumes of acetonitrile were added to one volume of plasma to precipitate out the proteins. The samples were vortexed for 20 s and placed on ice. Immediately after, they were centrifuged at 5,000 g for 10 min and again at 10,000 g for 2 min. The resulting supernatants were transferred to Eppendorf tubes and centrifuged for 2 min at 10,000 g. Finally, the supernatants were collected for analysis by LC-MS/MS to determine the targeted compound concentration. Percentage recovery was determined as follows: known concentrations of the compound were spiked into the blank plasma and acetonitrile was added (3:1 ratio) to precipitate out the proteins. Samples were vortexed for 20 s and placed on ice. The resulting supernatants were isolated via centrifugation as described above. The targeted compounds were identified using LC-MS/MS to detect their characteristic ions. Plasma concentrations at each point were plotted as a function of time.

Brain samples were collected at the same time points as described for blood samples. Male NMRI mice were overdosed with dolethal via i.p. administration. Mice were perfused with 0.9% saline and brains were collected in Eppendorf tubes. Brains were weighed, whereafter 400 μL of acetonitrile was added to the samples. Brains were homogenized with a pellet mixer (VWR, EU article no. 431-0100) and immediately placed on ice. Homogenized samples were centrifuged at 5,000 g for 10 min and again at 10,000 g for 2 min. The resulting supernatants were transferred to Eppendorf tubes and centrifuged for 2 min at 10,000 g. Finally, the supernatants were collected for analysis by LC-MS/MS to determine the targeted compound concentration in the brain tissue.

### Example 29

### Pharmacokinetic Analysis of Compounds 3.3 and 10.1

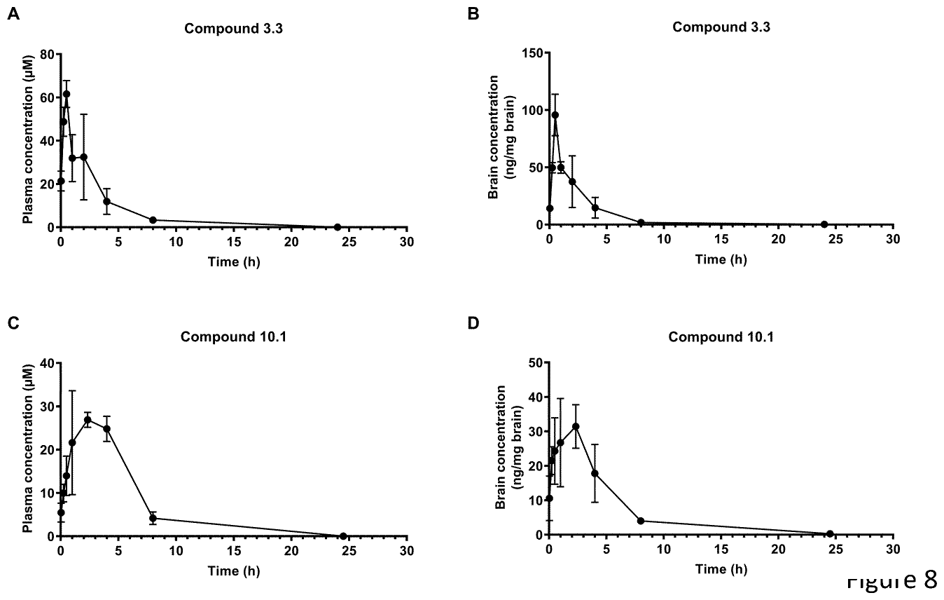

Pharmacokinetic analysis of compounds 3.3 and 10.1 was performed at 2 min, 15 min, 30 min, 1 h, 2-2.5 h, 4 h, 8 h, and 24 h after i.p. administration in mice at a dose of 300 mg/kg (FIG. 8), as described in example 28. The compound concentrations in mouse plasma and brain were determined by LC-MS/MS. The plasma and brain concentration of compound 3.3 peaked after 30 min with a maximal concentration (Cmax, mean (±SD)) of 62 (±6) μM in plasma and 96 (±18) ng/mg in the brain (FIG. 8A-B). The plasma concentration of compound 10.1 peaked after 1-4 h with a Cmax (mean (±SD)) of 27 (±2) μM after 2.5 h (FIG. 8C). Finally, the brain concentration of compound 10.1 peaked after 2.5 h with a Cmax (mean (±SD)) of 31 (±6) ng/mg (FIG. 8D).

### REFERENCES

1. Singh & Trevick. Neurol Clin. 2016; 34(4):837-47.
2. Ngugi et al. Epilepsia. 2010; 51(5):883-90.
3. Fisher et al. Epilepsia. 2014; 55(4):475-82.
4. Devinsky et al. Nat Rev Dis Primers. 2018; 4:18024.
5. Golyala & Kwan Seizure. 2017; 44:147-56.
6. Loscher et al. Nat Rev Drug Discov. 2013; 12(10):757-76.
7. Janmohamed et al. Neuropharmacology. 2020; 168:107790.
8. Franco et al. Pharmacol Res. 2016; 103:95-104.
9. Blond et al. Neurol Clin. 2016; 34(2):395-410, viii.
10. Loscher Neurochem Res. 2017; 42(7):1873-88.
11. Loscher Epilepsy Res. 2016; 126:157-84.
12. Barton et al. Epilepsy Res. 2001; 47(3):217-27.
13. Copmans et al. In: Pitkanen et al. Eds. Models of Seizures and Epilepsy. Second ed: Elsevier; 2017. p. 369-84.
14. Baraban & Loscher Adv Exp Med Biol. 2014; 813:283-94.
15. Doke & Dhawale Saudi Pharm J. 2015; 23(3):223-9.
16. Zhang et al. Sci Rep. 2017; 7(1):7195.
17. Leclercq et al. Epilepsy Behav. 2015; 45:53-63.
18. Baraban et al. Nature communications. 2013; 4:2410.
19. Lloyd et al. Adv Neurol. 1986; 44:1033-44.
20. Giometto et al. Lancet. 1998; 352(9126):457.
21. Peltola et al. Neurology. 2000; 55(1):46-50.
22. Errichiello et al. J Neuroimmunol. 2009; 211(1-2):120-3.
23. Errichiello et al. Neurol Sci. 2011; 32(4):547-50.
24. Liimatainen et al. Epilepsia. 2010; 51(5):760-7.
25. Falip et al. Eur J Neurol. 2012; 19(6):827-33.
26. Li et al. ACS Chem Neurosci. 2020; 11(5):730-42.
27. Copmans et al. ACS Chem Neurosci. 2018; 9(7):1652-62.
28. Sander et al. J Chem Inf Model. 2015; 55(2):460-73.
29. Mannhold et al. J Pharm Sci. 2009; 98(3):861-93.
30. Bowes et al. Nat Rev Drug Discov. 2012; 11(12):909-22.
31. Copmans et al. Mar Drugs. 2019; 17(11).
32. Siekierska et al. Nature communications. 2019; 10(1):708.
33. Scheldeman et al. Neurobiol Dis. 2017; 108:225-37.
34. Neckelmann et al. Behav Brain Res. 1996; 75(1-2):159-68.
35. Abbasi et al. Basic Clin Neurosci. 2017; 8(1):61-8.
36. Wang et al. Cogn Neurodyn. 2015; 9(3):291-304.
37. Hunyadi et al. J Neurosci Methods. 2017; 287:13-24.
38. Wilcox et al. Epilepsia. 2013; 54 Suppl 4:24-34.
39. Kehne et al. Neurochem Res. 2017; 42(7):1894-903.

1-31. (canceled)

32. A method for treating epilepsy, the method comprising administering to a subject in need thereof a compound of formula (I): 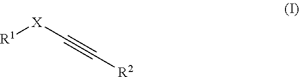
or a pharmaceutically acceptable salt of the compound of formula (I) in the form of a hydrate, solvate, or complex, where in formula (I):
X is selected from C═O, CH—OH, CH₂ and C═N—, with the proviso that if X is C═N—, the nitrogen is bound to a nitrogen atom of an R¹ substituent;
R¹ is selected from the group consisting of:
hydrogen, methyl, or a linear or branched C₂-C₄ alkyl;
an aromatic or aliphatic 5 membered ring, optionally comprising heteroatoms and/or optionally comprising further substituents;
an aromatic or aliphatic 6 membered ring, optionally comprising one or more heteroatoms and/or optionally comprising further substituents; and
a double 6 membered ring, wherein one or both rings are aromatic and optionally comprising one or more hetero atoms, and/or optionally comprising further substituents; and
R² is selected from the group consisting of:
phenyl, optionally substituted with OH, OCH₃, ethyl, halomethyl, one or more halogens, or with a linear or branched C₂-C₈ alkyl, where the C₂-C₈ alkyl is optionally further substituted with ═O or where a carbon atom in the C₂-C₈ alkyl is replaced with a halogen; and
a linear or branched C₁-C₁₀ alkyl in which a carbon atom is optionally replaced with a Si atom; and
a C₃-C₆ cycloalkyl.

33. The method of claim 32, wherein R¹ is phenyl, optionally substituted with —OH, —NO₂, —NH₂, —OCH₃, —OCH₂CH₃, —CH₂—NH₂, —CH₂—CH₂—NH₂, —F, or —Cl.

34. The method of claim 32, wherein R¹ is phenyl, substituted with a C₁-C₆, or C₁-C₄ alkyl wherein carbon is optionally replaced by oxygen and/or optionally comprises one or more —OH or ═O substituents.

35. The method of claim 32, wherein R¹ is phenyl, substituted with an aliphatic 6 membered ring, with optionally 1 or 2 hetero atoms.

36. The method of claim 32, wherein R¹ is an aromatic 5 membered ring, optionally comprising a sulfur heteroatom, or optionally comprising 1 or 2 nitrogen atoms.

37. The method of claim 32, wherein X is C═O, CH—OH, or CH₂.

38. The method of claim 32, wherein X is CH—OH, wherein R¹ is phenyl and R² is a phenyl substituted with methyl or a linear or branched C₂ to C₆ alkyl.

39. The method of claim 32, wherein the compound is rac-3-(4-(tert-butyl)phenyl)-1-phenylprop-2-yn-1-ol.

40. The method of claim 32, wherein X is C═N—, and the nitrogen of C═N— is bound to a nitrogen atom of R¹.

41. The method of claim 32, wherein X is C═N—, and wherein the compound comprises a pyrazolo [3,4-b] pyridine moiety or a indazole moiety.

42. The method of claim 32, wherein the compound is 3-((3-chlorophenyl)ethynyl)-1H-pyrazolo[3,4-b]pyridine.

43. The method of claim 32, wherein the epilepsy is a treatment-resistant epilepsy.

44. A compound having formula (I): 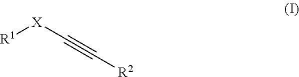
where:
X is selected from C═O, CH—OH, —CH₂ or C═N—, with the proviso that if X is C═N—, the nitrogen of the C═N— is bound to a nitrogen atom of an R¹ substituent;
R¹ is selected from the group consisting of:
hydrogen, methyl or a linear or branched C₂-C₄ alkyl;
an aromatic or aliphatic 5 membered ring, optionally comprising heteroatoms and/or optionally comprising further substituents;
an aromatic or aliphatic 6 membered ring, optionally comprising one or more heteroatoms and/or optionally comprising further substituents; and
a double 6 membered ring, wherein one or both rings are aromatic and optionally comprising one or more hetero atoms, and/or optionally comprising further substituents, and
R² is selected from the group consisting of:
phenyl, optionally further substituted with —OH, —OCH₃, ethyl, halomethyl, one or more halogens, or with a linear or branched C₂-C₈ alkyl, wherein the C₂-C₈ alkyl is optionally further substituted with ═O or wherein a carbon atom in the C₂-C₈ alkyl is replaced with a halogen;
a linear or branched C₁-C₁₀ alkyl, in which a carbon atom is optionally replaced with a Si atom; and
a C₃-C₆ cycloalkyl.

45. The compound of claim 44, wherein R¹ is phenyl, optionally substituted with —OH, —NO₂, —NH₂, —OCH₃, —OCH₂CH₃, —CH₂—NH₂, —CH₂—CH₂—NH₂, —F, or —Cl.

46. The compound of claim 44, wherein R¹ is phenyl, substituted with a C₁-C₆, or C₁-C₄ alkyl wherein a carbon atom is optionally replaced by oxygen and/or optionally comprises one or more —OH or ═O substituents.

47. The compound of claim 44, wherein R¹ is phenyl, substituted with aliphatic 6 membered ring, with optionally 1 or 2 hetero atoms.

48. The compound of claim 44, wherein R¹ is an aromatic 5 membered ring, optionally comprising a sulfur heteroatom, or optionally 1 or 2 nitrogen atoms.

49. The compound of claim 44, wherein X is selected from —C═O, —CH—OH, or —CH₂.

50. The compound of claim 44, wherein X is CH—OH, and wherein R¹ is phenyl and R² is a phenyl substituted with methyl or a linear or branched C₂-C₆ alkyl.

51. The compound of claim 44, wherein the compound is rac-3-(4-(tert-butyl)phenyl)-1-phenylprop-2-yn-1-ol.

52. The compound of claim 44, wherein X is C═N—, and the nitrogen of C═N— is bound to R¹ via a nitrogen atom of a substituent on a phenyl or pyridyl moiety.

53. The compound of claim 44, wherein X is C═N— and wherein the compound comprises a pyrazolo [3,4-b] pyridine moiety or a indazole moiety.

54. The compound of 44, wherein the compound is 3-((3-chlorophenyl)ethynyl)-1H-pyrazolo[3,4-b]pyridine.
